(19) United States
(12) Patent Application Publication
(10) Pub. No.: US 2025/0319158 A1
(43) Pub. Date: Oct. 16, 2025

(54) Compositions and Methods for Wound Healing

(71) Applicant: Gunesh Rajan, Udligenswil (CH)

(72) Inventors: Lee Yong Lim, Willetton (AU); Gunesh Pottayil Rajan, Daglish (AU); Leah Renee Benington, McKail (AU)

(21) Appl. No.: 18/682,526

(22) PCT Filed: Aug. 9, 2022
(86) PCT No.: PCT/AU22/50866

§ 371 Date: Feb. 9, 2024

(30) Foreign Application Priority Data
Aug. 9, 2021 (AU) 2021902450

(51) Int. Cl. A61K 38/18; A61K 47/18; A61K 47/38; A61K 47/42; A61L 27/26; A61L 27/54; A61P 17/02

(52) U.S. Cl. CPC A61K 38/1825; A61K 47/183; A61K 47/38; A61K 47/42; A61L 27/26; A61L 27/54; A61P 17/02; A61L 2300/252; A61L 2300/414; A61L 2430/34

(57) ABSTRACT

The invention relates to stabilized compositions comprising fibroblast growth factor 2. The present invention also relates to dosage forms and methods of treating wounds, including tympanic membrane perforations, by administering the composition to a patient in need thereof.

### FIELD OF THE INVENTION

The present invention relates to stabilized compositions comprising fibroblast growth factor 2. The present invention also relates to dosage forms and methods of treating wounds, including tympanic membrane perforations, by administering the composition to a patient in need thereof.

### BACKGROUND

The following discussion of the background art is intended to facilitate an understanding of the present invention only. The discussion is not an acknowledgement or admission that any of the material referred to is or was part of the common general knowledge as at the priority date of the application.

### A. Tympanic Membrane Perforations

Amongst children, chronic middle ear infection is reported to be the leading cause of mild to moderate hearing impairment, with tympanic membrane TM perforation a common co-morbidity or sequelae of the infection.

The TM is a thin, cone-shaped membrane which divides the external auditory canal from the middle ear. It is a unique structure, suspended between two air-filled cavities, which comprises of two distinctive regions—the pars flaccida and the pars tensa.

TM perforation is a hole or tear in the TM. Perforations of the TM can be classified according to the location, presence or absence of drainage and time to heal. Due to the nature of the healing process, acute, traumatic perforations are most likely to heal spontaneously, with complete closure reported in up to 90% of cases within 4 weeks. Conversely, chronic TM perforations show very poor rates of spontaneous closure and often require surgical intervention to achieve closure.

Rupture of the membrane may be caused by infection or trauma. Infection of the middle ear (otitis media) is one of the most common causes of TM perforation. Infection-mediated perforations are more commonly observed in children, developing countries and lower socio-economic populations of developed countries. The accumulation of exudate in the middle ear, as a result of infection, places pressure on the membrane causing it to bulge outwards. The central region of the TM may then become ischaemic, increasing its risk of perforation. Perforations as a result of uncomplicated otitis media are often small and demonstrate high rates of spontaneous healing following the resolution of the infection, with ongoing infection being the most common reason for an unresolved perforation.

Trauma is another common cause of TM perforation. Trauma to the membrane may be caused by changes in pressure (barotrauma) where the air pressure in the middle ear and the environmental air pressure are not balanced. Often this type of trauma occurs as a result of air travel, scuba diving or a direct blow to the head. The insertion of foreign objects into the ear such as cotton swabs or hairpins have been reported to cause TM perforation, as has a severe head trauma. Severe injuries to the head may result in the dislocation or damage of middle and inner ear structures, consequently causing the rupture of the TM. Loud sounds or blasts (acoustic trauma) may also cause a perforation of the TM in rare circumstances. Acoustic perforation of the TM may occur at volumes between 195 and 199 dB at 30° C., with higher sound pressures required when the frequency is low.

The insertion of tympanostomy (eustachian) tubes artificially creates a hole in the TM. Following the removal of these tubes, it is possible that the TM will not heal spontaneously due to a build-up of scar tissue around the margin of the perforation and the perforation could persist. Perforations may also be induced by heat, corrosives, lightning and water sports, with these causes demonstrating the lowest incidence of spontaneous closure. is thought that these perforations are less likely to heal naturally due to microscopic damage to the vasculature of the membrane, as a result of thermocoagulative effects, ultimately leading to necrosis.

In the majority of traumatic perforations (>90%), the TM heals spontaneously, usually within a few weeks. The process of spontaneous healing begins with the secretion of exudate at the edges of the perforation. This protects the damaged tissue from dehydration and provides support for the migration of new cells. Squamous epithelial cells proliferate and migrate to the site of perforation within days. The lamina propria is the slowest layer to be restored.

The closure of the perforation follows the natural pattern of epithelial migration for the TM. This healing pattern begins from the central portion of the perforation margin and continues to the periphery. Following perforation, mitotic activity throughout the pars tensa increases, particularly around the annulus and near the handle of the malleus. Shortly thereafter, this mitotic activity has been demonstrated to extend towards the perforation margin.

The size and time since perforation are indicators of healing time as the longer a perforation is present, the less likely it is to heal naturally, although spontaneous healing has been documented to occur as late as 10 months post perforation, with larger perforations taking a longer time to heal than small perforations. It has been suggested that the size and shape of the perforation may also correlate with the rate of healing, with a finding that large kidney-shaped perforations are least likely to heal without surgical intervention. It has been well established that age, nutritional status and immunity are important factors for the healing of cutaneous wounds and, as such, it is likely that these factors will also affect TM wound healing.

Chronic TM perforations are often characterised by inflammation, which may be localised or diffuse throughout the lamina propria and is associated with an increased number of inflammatory cells present in the membrane. Changes in the cellular organisation and composition of chronic TM perforations have also been observed, particularly at the border of the perforation where the external squamous epithelium either extends towards the inner surface of the perforation border or terminates at the perforation border. This same area may be covered by a thick layer of keratin, as the normal migration of keratinocytes is disturbed. This results in thickening of the perforation edge, which, on average, measures 114 μm compared with a normal membrane thickness of 30-90 μm. It is thought that this thickening and cellular disorganisation may be contributing factors in the failure of chronic

TM perforations to heal spontaneously.

Large or chronic TM perforations are currently managed with invasive surgical interventions such as myringoplasty or tympanoplasty. Both procedures use autologous, homologous or xenologous graft material, such as fascia or fat, to repair the perforation. Tympanoplasty additionally includes the repair of the ossicles. Although it is possible for the success rate of these procedures to be high (up to 94%), especially for small perforations, the outcome is often highly dependent upon the skill of the surgeon. Additionally, these procedures require the patient to undergo general anaesthesia, are time consuming, require sophisticated and expensive surgical equipment and setup, often require additional incisions to harvest graft material and the resulting membrane is often acoustically sub-optimal and prone to re-perforation. In some cases, multiple interventions are required to achieve a good outcome. Therefore, there is an established need for cost-effective, less invasive, and more reliable treatment alternatives, particularly in Australia, where it is likely that a large number of TM perforations remain untreated in remote Aboriginal communities.

B. Fibroblast growth factor (FGF-2)

Tissue formation during wound healing requires the orchestrated movement of cells to the wound site. A chemoattractant is defined as a chemical agent that induces cell migration toward itself. Chemoattractants are often members of the growth factor, cytokine and chemokine families. Cellular movement may occur by chemotaxis or chemokinesis in response to the presence of a chemoattractant. Chemotaxis is the movement of cells toward or away from a chemical gradient. Cells which are attracted toward the chemical gradient exhibit positive chemotaxis while repelled cells exhibit negative chemotaxis. Therefore, chemotaxis describes the directional movement of cells. Chemokinesis, on the other hand, is used to describe the random movement of cells in response to the presence of a chemoattractant.

Basic fibroblast growth factor (FGF-2) is an endogenous, 18 kDa heparin-binding protein. It is a growth factor and signaling protein encoded by the FGF2 gene. It is synthesized primarily as a 155 amino acid polypeptide, resulting in an 18 kDa protein. It promotes cellular proliferation, migration and differentiation, as well as angiogenesis in a variety of tissues, including skin, blood vessel, muscle, adipose, tendon/ligament, cartilage, bone, tooth, and nerve. Moreover, FGF-2 promotes the proliferation of a range of cell types, including endothelial, epithelial, preadipocyte, fibroblast and stem cells. This property is attractive in the context of wound healing where the tissue is non-homogenous, such as the tympanic membrane TM which is comprised of several cell types.

The density of FGF-2 receptors and subsequent responsiveness of various cells and tissues to external FGF-2 stimuli are likely to dictate the optimal FGF-2 dose for wound healing. The identification of an optimal FGF-2 dose for the repair of chronic TM perforations has likely been hindered by the poor stability of FGF-2 in solution. Most clinical studies investigating the use of FGF-2 for this indication have required the FGF-2 solution to be prepared in situ and there are reports that the bioactivity of FGF-2 is limited to 24-36 h in these formulations. Adverse effects as a result of the high FGF-2 doses and repeated applications required for membrane healing include secondary otitis media or reperforation of the membrane.

Chronic wounds often have reduced concentrations of growth factors, including FGF-2, resulting in reduced rates of healing and revascularisation of the wound. The decreased concentration of FGF-2 at chronic wound sites along with the many advantageous effects of FGF-2 on wound healing have led to extensive research and the investigation of new biomaterials and topical applications of FGF-2 for the treatment of chronic wounds. Although these treatments have shown some success with improved angiogenesis and tissue healing in vitro, the translation of this research into human trials has been limited. FGF-2 is quickly degraded during storage and upon delivery in vivo making its incorporation into a pharmaceutical product difficult.

The thermal- and heparin-dependent lability of the FGF-2 molecule in solution poses major challenges for the development of acceptable FGF-2 medicinal products. Commercially, lyophilisation is widely used to extend the shelf life of therapeutic proteins, and this has been applied to FGF-2. FGF-2 lyophilised with a cryoprotectant (e.g. glycine) is stable for up to 12 months storage at 4° C., and for up to 3 weeks at room temperature (<25° C.). Lyophilisation facilitates storage, shipping and transportation of the protein, but does little to mitigate its inherent instability once it is reconstituted into solutions. Binding of FGF-2 with its endogenous stabiliser, heparin, has been shown to improve its stability, however the inclusion of heparin as a FGF-2 stabiliser is not desirable in most clinical applications because the anticoagulant hardly qualifies as an inert pharmaceutical excipient.

The storage of reconstituted FGF-2 medicinal solutions at −20° C. is not a practical solution in clinical settings. Other alternatives, e.g. to reconstitute the lyophilised FGF-2 only when required, and applying a therapeutic regimen that requires the daily administration of multiple doses of FGF-2 to maintain pharmacological activity in vivo, are achievable only with highly compliant patients.

It is also not desirable for unstable FGF-2 solutions to be employed for the fabrication of medicinal products, e.g. tissue engineering constructs, due to the inevitable rapid decline in protein functionality during the manufacturing process. In order to achieve the desired FGF-2 load in the final product, the initial FGF-2 load must be high enough to compensate for the loss of FGF-2 functionality, as a result of protein instability, during manufacturing. The associated cost and safety implications of this approach may not be acceptable to both the manufacturer and the regulatory authorities.

There is a need in the art for effective stabilizing of FGF-2 aqueous solutions and the treatment of wounds and effective treatment and healing of tympanic membrane perforations. It is an objective of the invention to overcome one or more problems foreshadowed by the prior art.

### SUMMARY OF THE INVENTION

The present invention is directed to a stabilized FGF-2 formulation and to the use of that formulation in the treatment of wounds and, in particular, for healing tympanic membrane perforations and related disorders.

In a first aspect, the invention broadly resides in a composition comprising: (1) a fibroblast growth factor 2 (FGF-2), analog or variant thereof; and (2) a cellulose-based polymer, wherein said composition further comprises: an amino acid, or a serum albumin or an amino acid and a serum albumin.

Preferably, the cellulose-based polymer is methyl cellulose (MC), the amino acid is alanine and the serum albumin is human serum albumin.

In a second aspect, the invention provides a dosage form comprising the composition as described in the first aspect of this invention.

In a third aspect, the invention provides a method for treating a wound, wherein said method comprises the administration to a patient in need thereof a therapeutically effective amount of the dosage form as described in the second aspect of this invention.

Preferably, the wound is selected from the group consisting of: tympanic membrane perforations and chronic tympanic membrane perforations.

In a fourth aspect, the invention provides a device, wherein the device comprises: (1) the composition as described in the first aspect of this invention; and (2) a wound healing scaffold.

In a fifth aspect, the invention provides the use of a composition in the manufacture of a medicament for treating wounds, wherein said composition comprises: (1) a fibroblast growth factor 2 (FGF-2), analog or variant thereof; and (2) a cellulose based polymer and wherein said composition further comprises:
a. an amino acid;
b. a serum albumin; or
c. an amino acid and a serum albumin.

In a sixth aspect, the invention provides a method for stablising FGF-2, said method comprising preparing the composition as described in the first aspect of this invention.

Further features of the present invention are more fully described in the following description of several non-limiting embodiments thereof. This description is included solely for the purposes of exemplifying the present invention. It should not be understood as a restriction on the broad summary, disclosure or description of the invention as set out above.

### BRIEF DESCRIPTION OF THE DRAWINGS

Below is a brief description of each of the figures and drawings.

FIG. 1. Schematic representation of the methodology used to determine the storage stability of lyophilised and reconstituted FGF-2 solutions.

FIG. 2. Reproducibility of FGF-2 standard curves produced using commercial ELISA kits.

FIG. 3. Temperature stability of FGF-2 in water.

FIG. 4. Effect of excipients on the stability of FGF-2 aqueous solution (770 ng/ml) incubated at 25° C. for 2 h.

FIG. 5. Effect of excipients on the stability of FGF-2 aqueous solutions (770 ng/ml) incubated at 4° C. for 5 h, 25° C. for 5 h and 37° C. for 2 h.

FIG. 6 Effect of methylcellulose (MC) concentration on the stability of aqueous FGF-2 solutions (770 ng/ml) incubated at 4° C. for 5 h, 25° C. for 5 h and 37° C. for 2 h.

FIG. 7. Effect of excipient combinations on the stability of FGF-2 aqueous solutions (770 ng/ml) incubated at 4° C. for 5 h, 25° C. for 5 h and 37° C. for 2 h.

FIG. 8. Effect of excipient combinations on the stability of FGF-2 aqueous solutions (770 ng/ml) stored at 37° C. for up to 5 days.

FIG. 9. Effect of excipients on the stability of FGF-2 aqueous solutions (770 ng/ml) exposed to processing stressors.

FIG. 10. Stability of FGF-2 aqueous solutions (770 ng/ml) upon lyophilisation and storage.

FIG. 11. Effect of excipients on the stability of reconstituted FGF-2 aqueous solutions (770 ng/ml) over a 24 h period.

FIG. 12. Effect of excipients on the stability of reconstituted FGF-2 aqueous solutions (770 ng/ml) over a 7 day period.

FIG. 13 Schematic diagram showing the components of the transwell setup for the chemotactic migration assay.

FIG. 14. Cellular proliferation curves of primary human dermal fibroblasts in response to escalating doses (0.0098-200 ng/ml) of FGF-2 aqueous solutions containing different stabilisers.

FIG. 15. Wound healing capacity of stabilised FGF-2 solutions.

FIG. 16. Representative optical micrographs of simulated wounds in a human dermal fibroblast monolayer exposed to blank vehicles and FGF-2 solutions.

FIG. 17. Comparison of the number of human dermal fibroblasts which underwent chemotactic migration following 24 h exposure to stabilisation vehicles (1-6) in both the upper and lower chambers (A), or lower chamber only (B); or FGF-2 solutions (F1-F6) in both the upper and lower chambers (C), or lower chamber only (D) of a transwell set-up.

FIG. 18. Representative fluorescence micrographs of human dermal fibroblast cells which had undergone chemotactic migration to the basal surface of a transwell membrane in response to FGF-2.

FIG. 19. Diameter of blank prototype alginate scaffolds prepared using different vehicles to dissolve the alginate.

FIG. 20. Thickness of blank prototype alginate scaffolds prepared using different vehicles to dissolve the sodium alginate.

FIG. 21. Weight of blank prototype alginate scaffolds prepared using different vehicles to dissolve the alginate.

FIG. 22. Friability of blank prototype alginate scaffolds prepared using different vehicles to dissolve the alginate.

FIG. 23. The effect of different vehicles on the equilibrium hydration time of blank prototype alginate scaffold materials prepared using different vehicles to dissolve the alginate.

FIG. 24. Representative SEM micrographs of blank prototype alginate scaffolds prepared using different vehicles to dissolve the alginate.

FIG. 25. Analysis of the pore structure observed in blank prototype alginate scaffold materials prepared using different vehicles to dissolve the sodium alginate.

FIG. 26. Diameter of FGF-2 (1050 ng) loaded prototype alginate scaffolds prepared using different vehicles to dissolve the alginate.

FIG. 27. Thickness of FGF-2 (1050 ng) loaded prototype alginate scaffolds prepared using different vehicles to dissolve the sodium alginate.

FIG. 28. Weight of FGF-2 (1050 ng) loaded prototype alginate scaffolds prepared using different vehicles to dissolve the sodium alginate.

FIG. 29. Friability of FGF-2 (1050 ng) loaded prototype alginate scaffolds prepared using different vehicles to dissolve the sodium alginate.

FIG. 30. The effect of FGF-2 (1050 ng) loading on the equilibrium hydration time of prototype alginate scaffold materials prepared using different vehicles to dissolve the alginate.

FIG. 31. Representative SEM micrographs of FGF-2 (1050 ng) loaded prototype alginate scaffolds prepared using different vehicles to dissolve the sodium alginate.

FIG. 32. Analysis of the pore structure observed in FGF-2 (1050 ng) loaded prototype alginate scaffold materials prepared using different vehicles to dissolve the sodium alginate.

FIG. 33. Schematic diagram of the functional assay of FGF-2 loaded scaffolds.

FIG. 34. Comparison of cellular proliferation curves in response to escalating doses (2.3-150 ng/ml) of FGF-2 aqueous solutions containing different stabilisers.

FIG. 35. Cumulative release of FGF-2 from scaffold materials.

FIG. 36. Comparison of the cytoproliferative effects produced when murine (A) and human (B) fibroblast cells were exposed to FGF-2-loaded (1050 ng) scaffold materials.

FIG. 37. Representative stained images of live/dead cells in the interaction between murine fibroblast cells and scaffold materials.

FIG. 38. Biocompatibility of scaffold materials as measured by number of live cells interacting with the scaffold materials.

FIG. 39. Cytotoxicity of scaffold materials as measured by number of dead cells interacting with the scaffold materials.

### DETAILED DESCRIPTION OF THE INVENTION

For convenience, the following sections generally outline the various meanings of the terms used herein. Following this discussion, general aspects regarding compositions, use of medicaments and methods of the invention are discussed, followed by specific examples demonstrating the properties of various embodiments of the invention and how they can be employed.

Those skilled in the art will appreciate that the invention described herein is susceptible to variations and modifications other than those specifically described. The invention includes all such variations and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness. None of the cited material or the information contained in that material should, however be understood to be common general knowledge.

Manufacturer's instructions, descriptions, product specifications, and product sheets for any products mentioned herein or in any document incorporated by reference herein, are hereby incorporated herein by reference, and can be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

### 1. Definitions

The meaning of certain terms and phrases used in the specification, examples, and appended claims, are provided below. If there is an apparent discrepancy between the usage of a term in the art and its definition provided herein, the definition provided within the specification shall prevail.

Other than in the operating examples, or where otherwise indicated, all numbers expressing quantities of ingredients or reaction conditions used herein should be understood as modified in all instances by the term “about.” The term “about” when used in connection with percentages can mean±1%.

The invention described herein may include one or more range of values (e.g. size, concentration etc.). A range of values will be understood to include all values within the range, including the values defining the range, and values adjacent to the range which lead to the same or substantially the same outcome as the values immediately adjacent to that value which defines the boundary to the range. For example, a person skilled in the field will understand that a 10% variation in upper or lower limits of a range can be totally appropriate and is encompassed by the invention. More particularly, the variation in upper or lower limits of a range will be 5% or as is commonly recognised in the art, whichever is greater.

In this application, the use of the singular also includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

Throughout this specification, unless the context requires otherwise, the word “comprise” or variations such as “comprises” or “comprising”, will be understood to imply the inclusion of a stated integer or group of integers but not the exclusion of any other integer or group of integers.

“Therapeutically effective amount” as used herein with respect to methods of treatment and in particular drug dosage, shall mean that dosage that provides the specific pharmacological response for which the drug is administered in a significant number of subjects in need of such treatment. It is emphasized that “therapeutically effective amount,” administered to a particular subject in a particular instance will not always be effective in treating the diseases described herein, even though such dosage is deemed a “therapeutically effective amount” by those skilled in the art. It is to be further understood that drug dosages are, in particular instances, measured as oral dosages, or with reference to drug levels as measured in blood. Amounts effective for such a use will depend on: the desired therapeutic effect; the potency of the biologically active material; the desired duration of treatment; the stage and severity of the disease being treated; the weight and general state of health of the patient; and the judgment of the prescribing physician. Treatment dosages need to be titrated to optimize safety and efficacy. One skilled in the art will appreciate that the appropriate dosage levels for treatment will thus vary depending, in part, upon the indication for which the active agent is being used, the route of administration, and the size (body weight, body surface or organ size) and condition (the age and general health) of the patient. Accordingly, the clinician may titre the dosage and modify the route of administration to obtain the optimal therapeutic effect. A typical dosage may range from about 0.1 μg/kg to up to about 100 mg/kg or more, depending on the factors mentioned above. In other embodiments, the dosage may range from 0.1 μg/kg up to about 100 mg/kg; or 1 μg/kg up to about 100 mg/kg; or 5 μg/kg up to about 100 mg/kg.

The frequency of dosing will depend upon the pharmacokinetic parameters of the active agent and the formulation used. Typically, a clinician will administer the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

As used herein “pharmaceutically acceptable carrier” includes any and all solvents, dispersion media, coatings, antibacterial and antifungal agents, isotonic and absorption delaying agents, and the like that are physiologically compatible. Preferably, the carrier is suitable for topical administration into the ear.

As used herein the term “subject” generally includes mammals such as: humans; farm animals such as sheep, goats, pigs, cows, horses, llamas; companion animals such as dogs and cats; primates; birds, such as chickens, geese and ducks; fish; and reptiles. The subject is preferably human.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Features of the invention will now be discussed with reference to the following non-limiting description and examples.

### 2. Embodiments

### Composition

The present invention provides a composition comprising: (1) a fibroblast growth factor 2 (FGF-2), analog or variant thereof; and (2) a cellulose-based polymer, wherein said composition further comprises: an amino acid, or a serum albumin or an amino acid and a serum albumin.

For example, the composition comprises: (1) a fibroblast growth factor 2 (FGF-2), analogue or variant thereof; and (2) a cellulose-based polymer, wherein said composition further comprises: an amino acid. In an alternative example, the composition comprises: (1) a fibroblast growth factor 2 (FGF-2), analogue or variant thereof; and (2) a cellulose-based polymer, wherein said composition further comprises: a serum albumin. In a further alternative example, the composition comprises: (1) a fibroblast growth factor 2 (FGF-2), analog or variant thereof; and (2) a cellulose-based polymer, wherein said composition further comprises: an amino acid and a serum albumin.

In a preferred embodiment, the cellulose-based polymer contains a methoxyl group. More preferably, the cellulose-based polymer is methyl cellulose (MC). In one example, the cellulose-based polymer is not hydroxypropyl methylcellulose (HMPC). In a further example, the cellulose-based polymer is not hydroxypropyl cellulose (HPC). In yet a further example, the cellulose-based polymer is not carboxymethylcellulose (CMC).

In a further preferred embodiment, the composition is selected from the group consisting of: a therapeutic composition; a pharmaceutical composition; a cosmetic composition; and a veterinary composition.

Therapeutic compositions are within the scope of the present invention. Preferably the compositions are combined with a pharmaceutically acceptable carrier or diluent to produce a pharmaceutical composition (which may be for human or animal use). Suitable carriers and diluents include isotonic saline solutions, for example phosphate-buffered saline. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. See, e.g., Remington's Pharmaceutical Sciences, 19th Ed. (1995, Mack Publishing Co., Easton, Pa.) which is herein incorporated by reference.

The pharmaceutical composition can contain formulation materials for modifying, maintaining or preserving, for example, the pH, osmolarity, viscosity, clarity, colour, isotonicity, odour, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulphite or sodium hydrogen-sulphite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin), fillers; monosaccharides, disaccharides; and other carbohydrates (such as glucose, mannose, or dextrins); proteins (such as serum albumin, gelatin or immunoglobulins); colouring, flavouring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapol); stability enhancing agents (sucrose or sorbitol); tonicity enhancing agents (such as alkali metal halides, preferably sodium or potassium chloride), delivery vehicles, diluents, excipients and/or pharmaceutical adjuvants.

The optimal pharmaceutical composition will be determined by one skilled in the art depending upon, for example, the intended route of administration, delivery format, and desired dosage. Such compositions may influence the physical state, stability, rate of in vivo release, and rate of in vivo clearance of the FGF-2 of the invention. The preferred form of the pharmaceutical composition depends on the intended mode of administration and therapeutic application.

The primary vehicle or carrier in a pharmaceutical composition is aqueous in nature. For example, a suitable vehicle or carrier may be water for injection, physiological saline solution, possibly supplemented with other materials. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefor. In one embodiment of the present invention, pharmaceutical compositions may be prepared for storage by mixing the selected composition having the desired degree of purity with optional formulation agents in the form an aqueous solution.

The formulation components are present in concentrations that are acceptable to the site of administration. For example, buffers are used to maintain the composition at physiological pH or at a slightly lower pH, typically within a pH range of from about 5 to about 8.

Additional pharmaceutical compositions will be evident to those skilled in the art, including formulations of the invention in sustained- or controlled-delivery formulations. Techniques for formulating a variety of other sustained- or controlled-delivery means, such as liposome carriers, bio-erodible microparticles or porous beads and depot injections, are also known to those skilled in the art. Additional examples of sustained-sustained-release preparations include semipermeable polymer matrices in the form of shaped articles, for example, films, or microcapsules. Sustained release matrices may include polyesters, hydrogels, polylactides, copolymers of L-glutamic acid and gamma ethyl-L-glutamate, ethylene vinyl acetate or poly-D(−)-3-hydroxybutyric acid. Sustained-release compositions may also include liposomes, which can be prepared by any of several methods known in the art.

The pharmaceutical composition to be used for in vivo administration typically must be sterile. This may be accomplished by filtration through sterile filtration membranes. In addition, the compositions generally are placed into a container having a sterile access port. Once the pharmaceutical composition has been formulated, it may be stored in sterile vials as a solution.

In yet a further preferred embodiment, the FGF-2 is selected from the group consisting of: human FGF-2; bovine FGF-2; porcine FGF-2; and murine FGF-2. Preferably, the FGF-2 is recombinant.

In yet a further preferred embodiment, the analog or variant of FGF-2 has an amino acid sequence homology to human FGF-2 selected from the group consisting of: at least 75% sequence homology; at least 80%; at least 85%; at least 90%; at least 95%; at least 96%; at least 97%; at least 98%; and at least 99%.

The term “% sequence homology”, as used here, may for example be calculated as follows. The query sequence is aligned to the target sequence using the CLUSTAL W algorithm (Thompson et al, Nucleic Acids Research, 22:4673-4680 (1994)). A comparison is made over the window corresponding to one of the aligned sequences, for example the shortest. The window may in some instances be defined by the target sequence. In other instances, the window may be defined by the query sequence. The amino acid residues at each position are compared, and the percentage of positions in the query sequence that have identical correspondences in the target sequence is reported as % sequence homology.

Variants of FGF-2 include a polypeptide substantially homologous to FGF-2, but which has an amino acid sequence different from that of the FGF-2 sequence because one or more amino acids have been chemically modified or substituted by amino acids analogs. Preferably, any changes to the FGF-2 amino acid sequence to create a variant of FGF-2 can also include, in addition to amino acid substitutions, amino acid deletions and/or amino acid additions.

Amino acid substitutions are preferably conservative amino acid substitutions known to those skilled in the art. For example, the person skilled in the art may perform an amino acid substitution by selecting an amino acid from within the same class of amino acid that is shared with the specific amino acid that is identified for substitution. Examples of suitable amino acid substitutions are presented in Table 1 below.

TABLE 1

| Amino acids | Examples of conservative substitutions |
|---|---|
| Ala (A) | Val, Leu, Ile |
| Arg (R) | Lys, Gln, Asn |
| Asn (N) | Gln |
| Asp (D) | Glu |
| Cys (C) | Ser, Ala |
| Gln (Q) | Asn |
| Glu (E) | Asp |
| Gly (G) | Pro, Ala |
| His (H) | Asn, Gln, Lys, Arg |
| Ile (I) | Leu, Val, Met, Ala, Phe, Norleucine |
| Leu (L) | Ile, Val, Met, Ala, Phe, Norleucine |
| Lys (K) | Arg, Gln, Asn |
| Met (M) | Leu, Ile, Phe |
| Phe (F) | Leu, Val, Ile, Ala, Tyr |
| Pro (P) | Ala, Gly |
| Ser (S) | Thr, Ala, Cys |
| Trp (W) | Phe, Tyr |
| Thr (T) | Ser |
| Tyr (Y) | Trp, Phe, Thr, Ser |
| Val (V) | Ile Met, Leu, Phe, Ala, Norleucine |

In yet a further preferred embodiment, the amino acid has a hydrophobic side chain.

In yet a further preferred embodiment, the amino acid has no net charge

Preferably, the amino acid is selected from the group consisting of: alanine, valine, isoleucine, leucine, methionine, phenylalanine, tyrosine, tryptophan, glycine. Most preferably, the amino acid is alanine.

In yet a further preferred embodiment, the serum albumin is selected from the group consisting of: bovine serum albumin; and human serum albumin. Preferably, the serum albumin is human serum albumin.

In yet a further preferred embodiment, the FGF-2 is present at a concentration selected from the group consisting of: between 1 ng/ml to 5 mg/ml; between 10 ng/ml to 2 mg/ml; between 100 ng/ml to 1 mg/ml; between 200 ng/ml to 800 ng/ml; and 770 ng/ml.

In yet a further preferred embodiment, the MC is present at a concentration selected from the group consisting of: between 0.001 to 10%; between 0.01 to 10%; between 0.01 to 5%; between 0.01% to 1%; and 0.05% w/v. Preferably, the MC is at a concentration selected from the group consisting of: 0.05%; 0.072%; 0.1% w/v; and 0.5% w/v.

In yet a further preferred embodiment, the alanine is present at a concentration selected from the group consisting of: between 1 to 500 mM; and between 10 to 100 mM. Preferably, the alanine is at a concentration selected from the group consisting of: 20 mM; 28.902 mM; 50 mM; and 100 mM.

In yet a further preferred embodiment, the serum albumin is at a concentration selected from the group consisting of: between 0.1 to 100 mg/ml; between 0.5 to 50 mg/ml; and between 1 mg/ml to 10 mg/ml. Preferably, the serum albumin is at a concentration selected from the group consisting of: 1 mg/ml, 1.445 mg/ml; and 10 mg/ml.

In yet a further preferred embodiment, the composition further comprises water.

In yet a further preferred embodiment, the composition is a liquid, such as an aqueous solution.

In yet a further preferred embodiment, the composition is a freeze dried composition.

In yet a further preferred embodiment, the composition is a liquid composition, reconstituted from a freeze dried composition.

In yet a further preferred embodiment, further comprises a pharmaceutically acceptable carrier.

In yet a further preferred embodiment, the composition is adapted for wound healing. Preferably, the composition is adapted for tissue growth and repair.

In yet a further preferred embodiment, the composition further comprises a component selected from the group consisting of: mannitol; glucose; maltodextrin; HPMC; alginate; glycine; and NaCl. In an alternative embodiment, the composition does not comprise a component selected from the group consisting of: mannitol; glucose; maltodextrin; HPMC; alginate; glycine; and NaCl.

In yet a further preferred embodiment, the composition is stable and protects the FGF-2 from destabilizing forces. In one embodiment, the stabilisation of the composition is assessed using a method selected from the group consisting of: high performance liquid chromatography quantification; Western Blot; ELISA quantification; ELISA dose response assay, ELISA wound healing assay; chemotactic migration assay; thermal stability study based on temperature stressors; processing stability study based on freeze/thaw cycles; storage stability both as lyophilised dry power and upon reconstitution; a functional assay; and a quantification assay.

In yet a further preferred embodiment, the composition protects the FGF-2 against degradation selected from the group consisting of: physical degradation; UV degradation; thermal degradation; chemical degradation; and enzymatic degradation. Preferably, the composition protects the FGF-2 against degradation caused by process steps selected from the group consisting of: freeze/thaw cycles; and lyophilisation. Preferably, the composition protects the FGF-2 against loss in biological activity and the activity, as measured by ELISA, does not differ from the baseline value (t=0) by greater than 10%. Preferably, the composition protects the FGF-2 against loss in FGF-2 quantity, as measured with ELISA, did not differ from the baseline value (t=0) by greater than 10%. Most preferably, the composition protects the FGF-2 against loss in biological activity and the activity does not differ from the baseline value (t=0) by greater than 10%, wherein the biological activity is measured by fibroblast chemotactic migration assessed using the Boyden well chamber technique. In an alternative preferred embodiment, the composition protects the FGF-2 against loss in biological activity and the activity does not differ from the baseline value (t=0) by greater than 10%, wherein the biological activity is measured by a simulated wound of the fibroblast monolayer used to assess the wound healing capacity of FGF-2. In an alternative preferred embodiment, the composition protects the FGF-2 against loss in biological activity and the activity does not differ from the baseline value (t=0) by greater than 10%, wherein the biological activity is measured by a cellular proliferation assay using human dermal fibroblasts.

In yet a further preferred embodiment, the FGF-2 retains its effective biological activity for a period selected from the group consisting of; greater than 24 hours; greater than 36 hours; and greater than 48 hours. Preferably, the composition is stable for periods selected from the group consisting of: 6 months, 1 year and 2 years. In one example, the composition is stable at temperatures selected from the group consisting of: −4° C., 4° C., 18° C. and 25° C.

Therapeutic compositions are within the scope of the invention.

### Dosage Form

Dosage forms are within the scope of the invention. In a preferred embodiment, the invention provides a dosage form comprising the composition as described in the first aspect of this invention.

In a further preferred embodiment, the dosage form comprises a dose of FGF-2 selected from the group consisting of: between 1 ng to 5 ng; between 1 ng to 10 ng; between 1 ng to 100 ng; between 200 ng to 800 ng; 770 ng; 1 ng to 5 μg; between 10 ng to 2 μg; between 100 ng to 1 μg; 1 ng to 5 mg; between 10 ng to 2 mg; between 100 ng to 1 mg; between 5 mg to 150 mg of FGF-2; between 10 mg to 100 mg, between 20 mg to 75 mg, between 25 mg to 50 mg, and between 30 mg to 40 mg.

Preferably, the dosage form is stored in a sealed and sterile container.

Preferably, the scaffold material is biocompatible. Preferably, the biocompatibility of the scaffold materials is evaluated using a method selected from the group consisting of: a live/dead cytotoxicity/viability assay; and functional assay.

### Method for Treating

Method for treating a wound are within the scope of the invention. In a preferred embodiment, the invention provides a method for treating a wound, wherein said method comprises the administration to a patient in need thereof a therapeutically effective amount of the dosage form as described in the second aspect of this invention.

In a further preferred embodiment, the dosage form is administered at an amount to at least partially repair the wound.

In one embodiment, the wound is a perforation, burn, abrasion, cut tear or ulcer in need of increased proliferation and/or migration of fibroblasts to the site of the wound to at least partially repair the wound.

In a further preferred embodiment, the wound is selected from the group consisting of: tympanic membrane perforations and chronic tympanic membrane perforations.

In yet a further preferred embodiment, the dosage form administered to the patient in need thereof comprises a dose of FGF-2 selected from the group consisting of: between 1 ng to 5 ng; between 1 ng to 10 ng; between 1 ng to 100 ng; between 200 ng to 800 ng; 770 ng; 1 ng to 5 μg; between 10 ng to 2 μg; between 100 ng to 1 μg; 1 ng to 5 mg; between 10 ng to 2 mg; between 100 ng to 1 mg; between 5 mg to 150 mg; between 10 mg to 100 mg, between 20 mg to 75 mg, between 25 mg to 50 mg, and between 30 mg to 40 mg.

In yet a further preferred embodiment, the dosage form is administered to the subject utilising a dosing regimen selected from the group consisting of: at a frequency to repair the wound; twice hourly; hourly; once every six hours; once every 8 hours; once every 12 hours; once daily; twice weekly; once weekly; once every 2 weeks; once every 6 weeks; once a month; every 2 months; every 3 months; once every 6 months; and once yearly.

In yet a further preferred embodiment, the dosage form is administered topically to the site of the wound.

In yet a further preferred embodiment, the dosage form is administered to the site of the wound together with a wound healing scaffold.

In one preferred embodiment, the wound healing scaffold is applied to the wound before the dosage form is applied.

In an alternative preferred embodiment, the wound healing scaffold is applied to the wound concurrently when the dosage form is applied. In a further alternative preferred embodiment, the dosage form is applied to the wound healing scaffold before application to the site of the wound.

In yet a further preferred embodiment, the dosage form is administered via an applicator.

In yet a further preferred embodiment, the closure rate of tympanic membrane perforations is increased compared to conventional methods of treatment in the art.

Preferably, the tympanic membrane perforations is closed within a time period selected from: within 1 week of commencement of treatment; within 2 weeks of commencement of treatment; within 3 weeks of commencement of treatment; within 4 weeks of commencement of treatment; within 5 weeks of commencement of treatment; within 6 weeks of commencement of treatment; within 7 weeks of commencement of treatment; within 8 weeks of commencement of treatment; within 3 months of commencement of treatment; within 4 months of commencement of treatment; within 5 months of commencement of treatment; and within 6 months of commencement of treatment.

A subject that can be treated with the invention will include humans as well as other mammals and animals.

The effect of the administered therapeutic composition can be monitored by standard diagnostic procedures.

### Device

Devices are within the scope of the invention. In a preferred embodiment, the invention provides a device, wherein the device comprises: (1) the composition as described in the first aspect of this invention; and (2) a wound healing scaffold.

In a further preferred embodiment, the composition is contained within or imbedded into the wound healing scaffold.

In yet a further preferred embodiment, the wound healing scaffold has properties selected from the group consisting of: biocompatible; biodegradable; mechanically stable; low degree of cytotoxicity; and serves as a guide for 3D tissue regeneration. Preferably, the wound healing scaffold provides sustained release of FGF-2. Preferably, the wound healing scaffold promotes cellular migration, ingression and/or proliferation.

In yet a further preferred embodiment, the wound healing scaffold is porous.

Preferably, the wound healing scaffold is a gelatine sponge. Alternatively, the gelatine sponge is Gelfoam.

In yet a further preferred embodiment, the wound healing scaffold is an alginate-based scaffold material. Preferably, the wound healing scaffold comprises sodium alginate.

More preferably, the sodium alginate is present at a concentration selected from the group consisting of: between 0.01 to 20%; between 1 and 10%; between 2 and 5%; and 2%. More preferably, the wound healing scaffold utilises a crosslinking agent. Most preferably, the cross linking agent is CaCl2. In one example, the CaCl2 is present at a concentration selected from the group consisting of: between 10 and 100 mM; between 20 and 70 mM; and 50 mM.

In yet a further preferred embodiment, the wound healing scaffold has a pore area selected from the group consisting of: between 10,000 and 30,000 μm2; between 15,000 and 25,000 μm2; and 20847.6 μm2. Preferably, the wound healing scaffold has a pore diameter selected from the group consisting of: between 1 to 500 μm; between 90-160 μm; between 50 and 150 μm; 115.4 μm; and 75.5 μm.

In yet a further preferred embodiment, wound healing scaffold has a porosity selected from the group consisting of: between 10 and 99%, between 60 and 90%; between 25 and 75%; 54.3%; and 66.7%.

In yet a further preferred embodiment, the FGF-2 retains its effective biological activity for a period selected from the group consisting of; greater than 24 hours; greater than 36 hours; greater than 48 hours.

In yet a further preferred embodiment, the FGF-2 is initially released from the wound healing scaffold over the first two days, followed by a slower release for an additional 2-14 days. Preferably, the release of FGF-2 reaches plateau by day 14. Preferably, the FGF-2 releases for at least 14 days.

In yet a further preferred embodiment, the alginate-based scaffold material produces a higher sustained release profile of FGF-2 compared to release from a Gelfoam® scaffold. Preferably, the alginate-based scaffold material has smaller pore size, lower porosity and higher potential of FGF-2 binding to alginate compared to Gelfoam® scaffolds.

In yet a further preferred embodiment, the FGF-2 is present in the wound healing scaffold at a concentration selected from the group consisting of: between 1 ng/ml to 5 mg/ml; between 2.3-9.4 ng/ml; between 10 ng/ml to 2 mg/ml; between 50 ng/ml; between 75-150 ng/ml; ≥75 ng/ml; 9.4-37.5 ng/ml; 100 ng/ml to 1 mg/ml; between 200 ng/ml to 800 ng/ml; and 770 ng/ml.

In yet a further preferred embodiment, the FGF-2 is present in wound healing scaffold (dry scaffold equivalent) at a concentration selected from the group consisting of: between 1 ng/ml to 5 ng/ml; between 1 ng/ml to 10 ng/ml; between 1 ng/ml to 100 ng/ml; between 200 ng/ml to 800 ng/ml; 770 ng/ml; 1 ng/ml to 5 μg/ml; between 10 ng/ml to 2 μg/ml; between 100 ng/ml to 1 μg/ml; 1 ng/ml to 5 mg/ml; between 10 ng/ml to 2 mg/ml; between 100 ng/ml to 1 mg/ml.

In yet a further preferred embodiment, the wound healing scaffold is adapted to seed and grow keratinocytes. In yet a further preferred embodiment, the wound healing scaffold is adapted to seed and grow fibroblasts. In yet a further preferred embodiment, the wound healing scaffold is adapted to seed and grow epithelial cells.

### Use of a Composition in the Manufacture of a Medicament

Uses are within the scope of this invention. In a preferred embodiment, the invention provides the use of a composition in the manufacture of a medicament for treating wounds, wherein said composition comprises: (1) a fibroblast growth factor 2 (FGF-2), analog or variant thereof; and (2) a cellulose based polymer and wherein said composition further comprises:
a. an amino acid;
b. a serum albumin; or
c. an amino acid and a serum albumin.

### Method for Stabilising

Methods for stabilizing the FGF-2 are within the scope of the invention. In a preferred embodiment the invention provides a method for stablising FGF-2, said method comprising preparing the composition as described in the first aspect of this invention.

In a further preferred embodiment, the said method protects FGF-2 against degradation.

In yet a further preferred embodiment, the FGF-2 retains its effective biological activity for a period selected from the group consisting of; greater than 24 hours; greater than 36 hours; greater than 48 hours.

The addition of approved pharmaceutical excipients to stabilise the FGF-2 solutions is preferred from a safety standpoint, as the simpler methodology is likely to produce a less variable outcome and the choice of excipient can be limited to those with Generally Regarded as Safe (GRAS) status. Excipients for the stabilisation of protein solutions can be classified into four broad categories: salts, sugars, polymers or protein/amino acids, based on their chemical properties and mechanism of action. Salts (e.g. chlorides, nitrates) stabilise the tertiary structure of proteins by shielding charges through ionic interactions. Sugars (e.g. glycerol, sorbitol, fructose, trehalose) increase the surface tension and viscosity of the solution to prevent protein aggregation. Similarly, polymers (e.g. polyethylene glycol, cellulose derivatives) stabilise the protein tertiary structure by increasing the viscosity of the solution to prevent protein aggregation and intra- and inter-molecular electrostatic interactions between amino acids in the protein. Proteins (e.g. human serum albumin) are able to stabilise the structure of other proteins through ionic, electrostatic and hydrophobic interactions. Similarly, small amino acids with no net charge, such as alanine and glycine, stabilise proteins through the formation of weak electrostatic interactions.

As discussed above, the medicaments of the present invention may include one or more pharmaceutically acceptable carriers. The use of such media and agents for the manufacture of medicaments is well known in the art. Except insofar as any conventional media or agent is incompatible with the pharmaceutically acceptable material, use thereof in the manufacture of a pharmaceutical composition according to the invention is contemplated. Pharmaceutical acceptable carriers according to the invention may include one or more of the following examples:
a. surfactants and polymers, including, however not limited to polyethylene glycol (PEG), polyvinylpyrrolidone, polyvinylalcohol, crospovidone, polyvinylpyrrolidone-polyvinylacrylate copolymer, cellulose derivatives, HPMC, hydroxypropyl cellulose, carboxymethylethyl cellulose, hydroxypropylmethyl cellulose phthalate, polyacrylates and polymethacrylates, urea, sugars, polyols, and their polymers, emulsifiers, sugar gum, starch, organic acids and their salts, vinyl pyrrolidone and vinyl acetate; and/or
b. binding agents such as various celluloses and cross-linked polyvinylpyrrolidone, microcrystalline cellulose; and/or (3) filling agents such as lactose monohydrate, lactose anhydrous, microcrystalline cellulose and various starches; and/or
c. filling agents such as lactose monohydrate, lactose anhydrous, mannitol, microcrystalline cellulose and various starches; and/or
d. lubricating agents such as agents that act on the increased ability of the dosage form to be ejected from the packaging cavity, and/or
e. sweeteners such as any natural or artificial sweetener including sucrose, xylitol, sodium saccharin, cyclamate, aspartame, and acesulfame K; and/or
f. flavouring agents; and/or
g. preservatives such as potassium sorbate, methylparaben, propylparaben, benzoic acid and its salts, other esters of parahydroxybenzoic acid such as butylparaben, alcohols such as ethyl or benzyl alcohol, phenolic chemicals such as phenol, or quarternary compounds such as benzalkonium chloride; and/or
h. buffers; and/or
i. diluents such as pharmaceutically acceptable inert fillers, such as microcrystalline cellulose, lactose, dibasic calcium phosphate, saccharides, and/or mixtures of any of the foregoing; and/or
j. absorption enhancer such as glyceryl trinitrate; and/or
k. other pharmaceutically acceptable excipients.

Medicaments of the invention suitable for use in animals and in particular in human beings typically must be sterile and stable under the conditions of manufacture and storage.

2a. Tympanic Membrane

The TM is a thin, cone-shaped membrane which divides the external auditory canal from the middle ear. The TM is oval in shape, with the horizontal axis (9-12 mm) measuring longer than the vertical axis (8.5-9 mm). It has a trilaminar structure widely accepted to vary in thickness between 30-90 μm, with an outer layer comprising of keratinising squamous epithelium; a middle fibrous layer (lamina propria) composed of collagen (types I, II and III) and fibroblasts, which provides mechanical strength and elasticity; and an inner layer of mucosal epithelium. The variation in thickness of the membrane is due to differences in the composition of the lamina propria between the two distinct regions of the TM, the pars tensa and pars flaccida.

The external surface of the pars tensa is comprised of 3-5 cell layers of epidermal keratinising squamous epithelium. The cells of this epidermal layer have the unique ability to migrate laterally, providing a mechanism for the self-cleaning function of the external auditory canal. Additionally, the basal layer of epidermal cells has the capacity for both DNA synthesis and mitosis. This basal layer also contains hemidesmosomes, very small structures comparable to focal adhesions, found in keratinocytes, which attach the bottom surface of the basal cells to the basement membrane and, in the case of the TM, the lamina propria. The basement membrane is principally comprised of collagen (type IV and VII), laminin, fibronectin, heparin sulfate proteoglycans, osteonectin and kalinin and it is thought that disruption of the epidermal-basement membrane interaction is caused by a loss of hemidesmosome-mediated adhesion between the layers.

The major function of the TM is to transmit sound via the ossicles to the inner ear. Sound waves create changes in acoustic pressure which are captured by the pinna (external ear) and directed towards the TM. The TM vibrates in response to these changes in acoustic pressure, with the vibrations transferred and amplified by a chain of ossicles within the middle ear to the fluid filled inner ear (cochlea). The movement of fluid within the cochlea stimulates mechanoreceptors in the auditory hair cells which in turn, via the release of neurotransmitters, stimulate the auditory nerve and allow the perception of sound. Thus, the TM plays a crucial role in the perception of sound.

2b. Gelfoam®

Gelfoam® is a water-insoluble, non-elastic, porous product prepared from purified porcine skin, gelatin USP granules and water for injection. Gelatin is a protein extracted from collagen found in the connective tissues of animals, mainly cows and pigs. Gelatin is widely used in pharmaceutical products and has the USA Food and Drug Administration (FDA) Generally Regarded as Safe (GRAS) status. The gelatin sponge product is commercially available and is currently used for its haemostatic properties in surgical procedures. It may be cut without fraying and is able to absorb and hold many times its weight of blood or other fluids within its interstices. In recent years, it has also been a target biomaterial for a number of tissue engineering projects due to its ability to be completely absorbed in vivo within a few months. Early clinical trials investigating the efficacy of FGF-2 for the healing of chronic TM perforations had utilised the Gelfoam® as a model scaffold material. In each of these studies, the Gelfoam® was cut into an appropriate size, soaked in an FGF-2 solution, and immediately placed into the perforation. The bioactivity of FGF-2 in these Gelfoam® pieces is likely limited to 24-36 hours, which is not long enough to ensure complete healing of the typical chronic TM perforation.

2c. Alginate

Alginic acid is a naturally occurring polysaccharide derived primarily from marine brown seaweed. It is a block co-polymer comprised of two monosaccharides, (1-4) linked β-D-mannuronic acid (M units) and α-L-guluronic acid (G units) which may be covalently linked together in different sequences. Its salt, sodium alginate (henceforth referred to as alginate).

Alginate is hydrophilic and, when dissolved in water, forms a viscous solution. In the presence of divalent ions, most commonly calcium, the divalent ions exchange with sodium ions on the G blocks, binding adjacent polymer chains together to form a hydrogel with an “egg-box” structure. This ion-exchange gelation process is commonly referred to as crosslinking and may occur even under very mild conditions, making this technique suitable for the incorporation of sensitive macromolecules, such as proteins and cells.

Alginate hydrogels are customisable, with alterations to the M/G ratio, molecular weight or concentration of the alginate, gelation rate, or composition and concentration of crosslinking solution all impacting the physical and mechanical properties of the hydrogel. For example, an increased alginate concentration, G content, crosslinking solution strength or crosslinking time could contribute to the production of a hydrogel with increased mechanical strength due to an increased number of crosslinks within the structure.

Alginate hydrogels are degraded through the exchange of the divalent crosslinking ions in the hydrogel with monovalent ions in the surrounding environment. This process is often unpredictable, leading to varied mechanical strength and cargo release profiles over time, however the degradation of alginate hydrogels may be modified by altering the crosslinking density, with a higher degree of crosslinking associated with slower hydrogel degradation.

The present invention will now be described with reference to the following non-limiting Examples. The description of the Examples is in no way limiting on the preceding paragraphs of this specification, however, is provided for exemplification of the methods and compositions of the invention.

### EXAMPLES

It will be apparent to persons skilled in the milling and pharmaceutical arts that numerous enhancements and modifications can be made to the above described processes without departing from the basic inventive concepts. For example, in some applications the biologically active material may be pretreated and supplied to the process in the pretreated form. All such modifications and enhancements are considered to be within the scope of the present invention, the nature of which is to be determined from the foregoing description and the appended claims. Furthermore, the following Examples are provided for illustrative purposes only, and are not intended to limit the scope of the processes or compositions of the invention.

### A Example 1—Stabilisation of Recombinant Human Basic Fibroblast Growth Factor (FGF-2) Against Thermal and Processing Stressors

### A.1 Study Aim

To identify stabilisation vehicles for basic fibroblast growth factor (FGF-2) and to evaluate the ability of these stabilisation vehicles to protect FGF-2 against physical stressors encountered in pharmaceutical processing.

### A.2 Materials and Methods

### A.2.1 Materials

Lyophilised, recombinant human FGF-2 was kindly provided by Essex Bio-Pharmaceutical Co (Zhuhai, China). Low viscosity sodium alginate was purchased from Buchi Labortechnik AG (Flawil, Switzerland), sodium chloride, glycine and mannitol were purchased from Ajax Finechem (NSW, Australia), maltodextrin M180 was sourced from the Grain Processing Corporation (Iowa, USA), D-glucose was purchased from Chem-Supply (South Australia, Australia), methylcellulose USP 4000 was purchased from Professional Compounding Chemists of Australia (PCCA; NSW, Australia), and human serum albumin, DL-alanine and hydroxypropyl methylcellulose (HPMC) were purchased from Sigma-Aldrich (Missouri, USA). Deionised water was used throughout and supplied by a BOSS water system (PSI Water Filters, Tasmania, Australia).

### A.2.2 Preparation and Quantification of FGF-2 Stock Solution

The lyophilised FGF-2 powder was reconstituted at 1 mg/ml (based on dry powder weight) in water, and the stock solution was stored at −20° C. (Westinghouse Freezer FJ302V-L, Westinghouse Electric Corporation, Pennsylvania, USA) as aliquots of 20 to 100 μl in 0.1 ml Eppendorf® tubes (Eppendorf, New York, USA).

To determine the functional FGF-2 content in the lyophilised powder, a freshly reconstituted FGF-2 stock solution was diluted on ice to 100 μg/ml (based on dry powder weight) and immediately analysed using a commercial ELISA kit (Human FGF Basic ELISA Kit, Thermo Fisher Scientific, Maryland, USA).

### A.2.3 Quantification of FGF-2 by ELISA

Samples were diluted with water to achieve a FGF-2 concentration of between 50 and 800 μg/ml and immediately assayed using a commercial ELISA kit according to the manufacturer's instructions. The absorbance at 450 nm obtained for the diluted FGF-2 solution, using a microplate reader (Polarstar Optima, BMG Labtech, Victoria, Australia), was translated to FGF-2 content using a standard curve. The standard curve was plotted from the absorbance readings of standard FGF-2 solutions (15.6-1000 μg/ml) prepared according to manufacturer's instructions using the FGF-2 standard provided in the ELISA kit.

### A.2.4 Preliminary Investigation of FGF-2 Stability

In order to assess FGF-2 stability in aqueous solutions, the FGF-2 stock solution was retrieved from storage at −20° C. and allowed to thaw at 4° C. (Westinghouse Refrigerator RP372V-R, Westinghouse Electric Corporation, Pennsylvania, USA) before being serially diluted to 1.7 ng/ml (functional FGF-2, as determined by ELISA) with water. The diluted FGF-2 stock solution (11.1 μl) was then added into 0.1 ml Eppendorf® tubes that had been pre-incubated at 4° C., 25° C. (Memmert Incubator UF160, In Vitro Technologies, Victoria, Australia) or 37° C. (Memmert Incubator UF160, In Vitro Technologies, Victoria, Australia) for 2 h with 48.9 μl of water, to give a final FGF-2 concentration of 315 μg/ml. All samples were prepared in quadruplicates. The tubes were returned to the respective incubation conditions and samples removed at timepoints ranging from 0 to 48 h were stored at −20° C. until required for analysis. The frozen samples were thawed at 4° C. then assayed for residual FGF-2 content using a commercial ELISA kit. FGF-2 aqueous solutions were deemed stable if the FGF-2 content, as measured with ELISA, did not differ from the baseline value (t=0) by greater than 10%.

### A.2.5 Survey Study of Potential Excipient Stabilisers for FGF-2 Solutions

FGF-2 stock solutions for all subsequent studies were prepared by reconstituting the lyophilised FGF-2 powder with water to a concentration of 1 mg/ml (based on dry powder weight), with the baseline active FGF-2 concentration of each stock solution determined by ELISA. Each FGF-2 stock solution was then diluted to a final FGF-2 stock concentration of 2.5 μg/ml (based on active FGF-2 present in the solution, confirmed with ELISA) with water prior to its use in the subsequent stabilisation studies.

Various excipients were evaluated for stabilisation effects on FGF-2 in aqueous solutions. Concentrated stock solutions of each potential stabilisation vehicle were prepared by dissolving the stated excipient in water, at the concentrations specified in Table 2. Each −4, 4 or 18° C. for up to 12 months vehicle stock was diluted with either water or FGF-2 stock solution (2.5 μg/ml), in a 0.1 ml Eppendorf® tube, to give the final excipient concentration indicated in Table 2. Vehicles diluted with water were used as blanks for ELISA analysis, while vehicles diluted with the FGF-2 stock solutions were treated as test samples. The final concentration of FGF-2 in the test samples was 770 ng/ml, which corresponded to the labelled FGF-2 concentration of the commercially available Beifushu™ eye drops (Zhuhai Essex Bio-Pharmaceutical Co, Zhuhai, China). The Beifushu™ product was shown to be effective for the treatment of chronic TM perforations (unpublished data from a study on paediatric patients led by Professor Gunesh Rajan in Perth, Western Australia), but required storage at refrigerated (2-8° C.) temperatures to maintain FGF-2 activity.

TABLE 2
Composition of potential FGF-2 stabilisation
vehicles used in the survey study.

| | Concentration of Excipient | | Final Excipient | |
|---|---|---|---|---|
| Excipient | in Vehicle Stock | | Concentration | |
| Sodium alginate | 2.890% | w/v | 2% | w/v |
| Sodium chloride (NaCl) | 1.301% | w/v | 0.9% | w/v |
| Maltodextrin | 1.445% | w/v | 1% | w/v |
| | 14.451% | w/v | 10% | w/v |
| Mannitol | 7.225% | w/v | 5% | w/v |
| | 14.451% | w/v | 10% | w/v |
| Glucose | 7.225% | w/v | 5% | w/v |
| | 14.451% | w/v | 10% | w/v |
| Methylcellulose (MC) | 0.145% | w/v | 0.1% | w/v |
| | 0.723% | w/v | 0.5% | w/v |
| Hydroxypropyl | 0.723% | w/v | 0.5% | w/v |
| methylcellulose (HPMC) | 2.890% | w/v | 2% | w/v |
| Human Serum Albumin | 1.445 | mg/ml | 1 | mg/ml |
| (HSA)* | 14.451 | mg/ml | 10 | mg/ml |
| Alanine | 28.902 | mM | 20 | mM |
| | 144.509 | mM | 100 | mM |
| Glycine | 28.902 | mM | 20 | mM |
| | 144.509 | mM | 100 | mM |

| | | |
|---|---|---|
| Water (as control) | N/A | N/A |

*Prepared immediately prior to use and maintained at 4° C. as per manufacturer's specifications.

Samples were stored at 25° C. for up to 2 h. At specified incubation times, triplicate samples were removed and stored at −20° C. until required for analysis. The frozen samples were thawed at 4° C. and assayed, neat as well as diluted 1 in 500 and 1 in 1000 with water (to give theoretical FGF-2 concentrations of 1.54 ng/ml and 770 μg/ml, respectively). FGF-2 aqueous solutions were deemed stable if the FGF-2 content, as measured with ELISA, did not differ from the baseline value (t=0) by greater than 10%.

### A.2.6 Optimisation of Stabilisation Vehicles

Based on the results of the stabilisation survey, glucose, methylcellulose (MC), human serum albumin (HSA) and alanine were chosen for further investigation, individually and in combination, to determine their ability to stabilise FGF-2 against thermal and other stressors encountered during the processing of FGF-2 for biomedical applications. Concentrated stock solutions of each potential stabilisation vehicle were prepared by dissolving the stated excipient in water, according to Table 3. Each vehicle stock was diluted with either water or FGF-2 stock solution (2.5 μg/ml), in a 0.1 ml Eppendorf® tube to give the final excipient concentration indicated in Table 3. Vehicles diluted with water were used as blanks for ELISA analysis, while vehicles diluted with the FGF-2 stock solutions were treated as test samples. The final concentration of FGF-2 in these samples was 770 ng/ml. To facilitate their description, the vehicles are identified by the specific IDs provided in Table 4, and the final FGF-2 solutions prepared using these vehicles are identified by the specific IDs provided in Table 5.

To determine the ability of the vehicles to protect FGF-2 against thermal degradation, samples were stored at 4° C. for 5 h, 25° C. for 5 h, 37° C. for 2 h and/or 37° C. for 5 days. At the specified incubation times, triplicate samples were removed and stored at −20° C. until they were analysed with the ELISA kit. Solutions were deemed stable if their FGF-2 content did not differ from the baseline value (t=0) by greater than 10%.

TABLE 3
Composition of potential FGF-2 stabilisation vehicles.

| | Concentration of Excipient | | Final Excipient | |
|---|---|---|---|---|
| Excipient(s) | in Vehicle Stock | | Concentration | |
| Glucose | 14.451% | w/v | 10% | w/v |
| Methylcellulose | 0.072% | w/v | 0.05% | |
| (MC) | 0.145% | w/v | 0.10% | w/v |
| | 0.723% | w/v | 0.50% | w/v |
| Human Serum | 1.445 | mg/ml | 1 | mg/ml |
| Albumin (HSA)* | | | | |
| Alanine | 28.902 | mM | 20 | mM |

| | | |
|---|---|---|
| MC and Alanine | MC 0.072% w/v and | MC 0.05% w/v and |
| | Alanine 28.902 mM | Alanine 20 mM |
| MC and HSA* | MC 0.720% w/v and | MC 0.05% w/v and |
| | HSA 1.445 mg/ml | HSA 1 mg/ml |
| MC and HSA and | MC 0.072% w/v, | MC 0.05% w/v, |
| Alanine* | HSA 1.445 mg/ml and | HSA 1 mg/ml and |
| | Alanine 28.902 mM | Alanine 20 mM |
| Water (Negative | N/A | N/A |
| Control) | | |

*Prepared immediately prior to use and maintained at 4° C. as per manufactures specifications for HSA.

TABLE 4
Identification key for FGF-2 stabilisation vehicles.

| Stabilisation | |
|---|---|
| Vehicle ID | Composition of Vehicle |
| 1 | Water (as control) |
| 2 | Methylcellulose (MC; 0.05% w/v) in water |
| 3 | Alanine (20 mM) in water |
| 4 | Human Serum Albumin (HSA; 1 mg/ml) in water |
| 5 | MC (0.05% w/v) and alanine (20 mM) in water |
| 6 | MC (0.05% w/v) and HSA (1 mg/ml) in water |
| 7 | MC (0.05% w/v), alanine (20 mM) and HSA |
| | (1 mg/ml) in water |

The capacity of the stabilisation vehicles to stabilise FGF-2 solutions against processing stressors was also investigated by exposing the samples to 3 repeated freeze/thaw cycles as follows: samples were frozen at −20° C. for 24 h then thawed over 30 min at 4° C. before being frozen again, with this process repeated a further two times. Samples were transferred to storage at −20° C. until required for analysis. The effects of lyophilisation were also studied. The FGF-2 solutions were frozen at −20° C. for 12 h then lyophilised over 24 h (Alpha 1-2 LDplus, Martin Christ Gefriertrocknungsanlagen GmbH, Osterode am Harz, Germany), with the lyophilised samples stored at −20° C. until required for analysis. Lyophilised samples were removed from storage and reconstituted with water to their pre-lyophilisation volume immediately prior to analysis. Samples were analysed neat as well as diluted 1 in 500 and 1 in 1000 with water (to give theoretical FGF-2 concentrations of 1.54 ng/ml and 770 μg/ml respectively) with the ELISA kit, and deemed stable if their FGF-2 content after processing did not differ from the baseline value by greater than 10%.

TABLE 5
Identification key for FGF-2 solutions.

| FGF-2 | |
|---|---|
| Solution | |
| ID | Description of Solution |
| F1 | Solution of FGF-2 (770 ng/ml) in water |
| F2 | Solution of FGF-2 (770 ng/ml) and MC (0.05% w/v) in water |
| F3 | Solution of FGF-2 (770 ng/ml) and alanine (20 mM) in water |
| F4 | Solution of FGF-2 (770 ng/ml) and HSA (1 mg/ml) in water |
| F5 | Solution of FGF-2 (770 ng/ml), MC (0.05% w/v) and alanine |
| | (20 mM) in water |
| F6 | Solution of FGF-2 (770 ng/ml), MC (0.05% w/v) and HSA |
| | (1 mg/ml) in water |
| F7 | Solution of FGF-2 (770 ng/ml), MC (0.05% w/v), alanine |
| | (20 mM) and HSA (1 mg/ml) in water |

### A.2.7 Storage Stability of Lyophilised and Reconstituted FGF-2 Solutions

F1, F5 and F6 solutions as described in Table 4 were prepared and separately aliquoted (50 μl) into 0.1 ml Eppendorf® tubes. Triplicate baseline samples were immediately frozen at −20° C. until required for analysis while all remaining samples were frozen at −20° C. for 24 h, then lyophilised over 24 h.

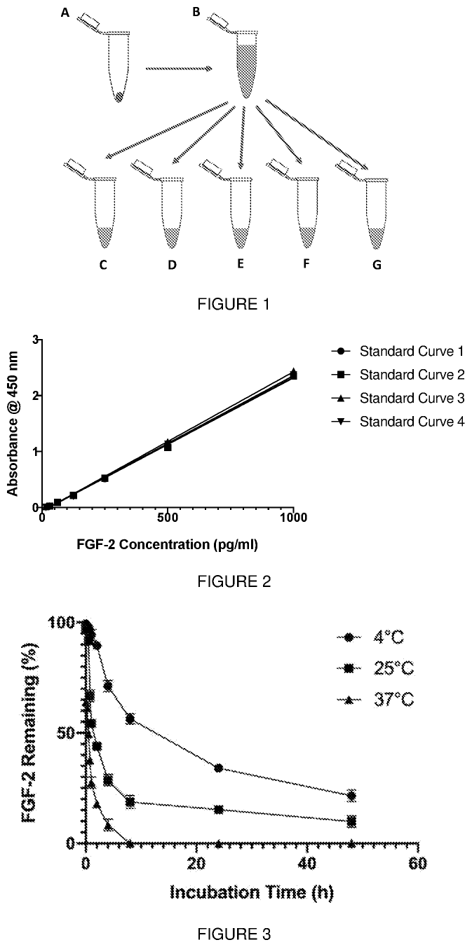

Triplicate lyophilised samples were stored at −4° C. (Refrigerator/Freezer GM-422FW, LG Electronics, Busan, South Korea), 4° C. or 18° C. (HR6WC30 Wine Fridge, Hisense, Qingdao, China) for up to 12 months. Temperatures were monitored weekly over the duration of the study using an internally mounted thermometer (SFL−10 to +110, Brannan Thermometers and Gauges, Cleator Moor, UK). At defined time points (time 0, 1 week, 2 weeks, 1, 3, 6, 9 and 12 months) triplicate lyophilised samples were reconstituted with 50 μl of water. Immediately following reconstitution, 10 μl of the resulting solution was transferred to storage at −20° C. until required for analysis. The remaining solution (40 μl) was then divided into 10 μl aliquots which were stored at 4° C. for 24 h and 7 days or 18° C. for 24 h and 7 days (FIG. 1) before these samples were also transferred to storage at −20° C. until required for analysis. All samples were assayed for residual FGF-2 content using the commercial ELISA kit. Samples were deemed stable if the FGF-2 content, as measured with ELISA, did not differ from the pre-lyophilisation value by greater than 10%. Refer to FIG. 1.

FIG. 1. Schematic representation of the methodology used to determine the storage stability of lyophilised and reconstituted FGF-2 solutions. Lyophilised FGF-2 samples (A) were stored at −4° C., 4° C. or 18° C. for up to 12 months. At defined time points, samples were reconstituted with 50 μl of water (B). The reconstituted FGF-2 solution was divided into 10 μl aliquots (C-G) with one aliquot (C) immediately transferred to storage at −20° C. until required for analysis. The remaining aliquots were stored at 4° C. for 24 h (D) or 7 days (E), or 18° C. for 24 h (F) or 7 days (G) before being transferred to storage at −20° C. until required for analysis.

### A.2.8 Data Analysis

Results are expressed as mean±SD. Data from the survey stabilisation study were analysed by one-way ANOVA. All other data were analysed by two-way ANOVA with post-hoc Tukey's test applied for paired comparison of means, unless stated otherwise. All statistical analyses were completed using GraphPad Prism 8 (California, USA) and a P value ≤0.05 was considered to be significant.

### A.3 Results

### A.3.1 Quantification of Functional FGF-2

Quantification of the inherently unstable FGF-2 in aqueous solutions posed a significant challenge. Although FGF-2 has reportedly been detected by high performance liquid chromatography (HPLC) or Western Blot, the limitations associated with protein quantification using these techniques made them inappropriate choices for this study. FGF-2 must retain the correct tertiary structure to maintain its biological activity. Protein quantification via Western Blot relies on denaturation of the protein to allow its movement through the gel and, as a result, the proportion of total protein retaining the correct tertiary structure is unable to be determined using this technique. HPLC performed using a specialty heparin-affinity column allows FGF-2, which has retained the correct tertiary structure, to be detected, however this technique has primarily been used to purify FGF-2 and therefore relies on concentrated protein solutions (up to 54 mg/ml). As a result, heparin-affinity HPLC is not sufficiently sensitive for the quantification of FGF-2 present in aqueous solutions at low concentrations. In order to detect and quantify biologically relevant concentrations (pg-μg/ml) of FGF-2, ELISA was identified as the most appropriate choice for this study. The ELISA kit chosen for this study requires FGF-2 to have retained the correct tertiary structure for antibody binding. Therefore, this assay specifically quantifies FGF-2 which has retained the active conformation.

The FGF-2 content of each stock solution was quantified immediately upon preparation in accordance with the protocol provided with the commercial ELISA kit. The standard curves produced using the FGF-2 standard, provided in the kit, were linear (R2 range: 0.9989-0.9990) and consistent (FIG. 2) across plates and analysis days, with no statistically significant differences detected between the slope values (multiple linear regression analysis; P=0.1136) or the intercept values (P=0.3119) of the regression lines obtained throughout the project (n=19).

FIG. 2. Every fourth standard curve produced throughout the study is presented to demonstrate the highly reproducible results obtained from the ELISA protocol.

Each data point represents the mean±SD (n=3).

### A.3.2 Preliminary Stability Study

The stability of FGF-2 in water before and after exposure to temperature stressors was determined in a pilot study. The solutions were deemed stable if the FGF-2 content, as measured with ELISA, did not differ from the baseline value of 770 ng/ml (t=0, 4° C.) by greater than 10%. The FGF-2 content of the solution decreased very rapidly when exposed to all 3 temperatures, with samples exposed to 4° C. and 25° C. remaining stable for only 2 h and 30 min, respectively. The time for the residual FGF-2 content to decrease to 50% baseline level was 30 min at 37° C., 1 h at 25° C. and 8 h at 4° C. (FIG. 3).

FIG. 3. Temperature stability of FGF-2 in water. FGF-2 solutions (315 pg/ml) were exposed to 4° C., 25° C. or 37° C. for up to 48 h. At specific time points, samples were withdrawn and analysed for residual FGF-2 content using an ELISA kit. Data are expressed as the percentage of baseline FGF-2 content (mean±SD, n=4).

### A.3.3 Identification of Potential Stabilisers for Aqueous FGF-2 Solutions

The ability of excipients, from each class of known protein stabilisers (salt, sugar, polymer or protein/amino acid), to stabilise the aqueous FGF-2 solution was investigated. The excipients were added to the FGF-2 solution at concentrations that have been reported in the published literature to be effective for the respective excipients to stabilise proteins. The resultant FGF-2 solutions were then stored at 25° C. for 2 h before they were analysed for FGF-2 content via the ELISA assay.

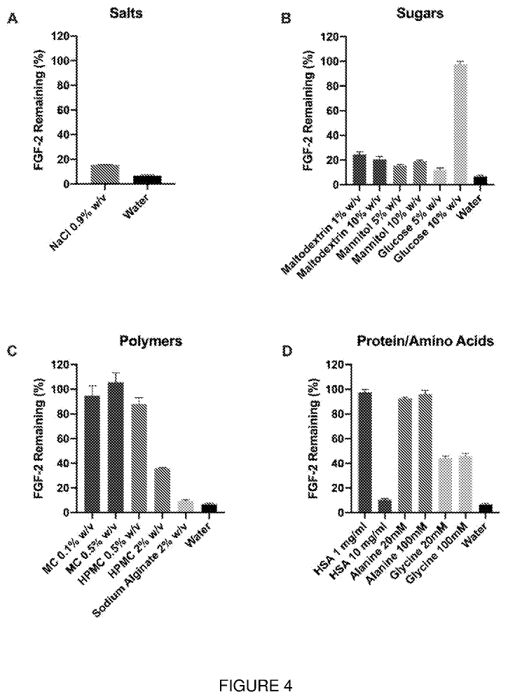

In the protein/amino acids class (FIG. 4D). HSA 1 mg/ml, alanine 20 mM and alanine 100 mM were able to stabilise FGF-2 at 25° C. for at least 2 h, retaining residual FGF-2 contents of 97.4, 92.7 and 96.1%, respectively. Increasing the concentration of HSA to 10 mg/ml did not correspond with a greater stabilisation effect; instead, this solution retained just 10.3% of the baseline FGF-2, similar to the control (FGF-2 in water, one-way ANOVA, P=0.2686). Glycine at 20 mM and 100 mM also failed to stabilise the FGF-2 solution, retaining 44.3% and 45.9% of the baseline FGF-2 after 2 h of exposure to 25° C.

Of the polymer excipients, only MC was able to successfully stabilise FGF-2 for at least 2 h at 25° C. (FIG. 4C). There was no difference (one-way ANOVA, P=0.1545) in the stabilisation effects between 0.1% (w/v) and 0.5% (w/v) MC solutions, which yielded 94.8 and 105.6%, respectively, of the baseline FGF-2 at 2 h. With the HPMC 0.5% w/v vehicle, 87.9% of the baseline FGF-2 was retained which, while statistically comparable to the residual FGF-2 content seen with the MC 0.1% w/v vehicle (P=0.5806), did not meet the criteria for successful FGF-2 stabilisation. HPMC 2% w/v and sodium alginate 2% w/v also failed to stabilise FGF-2, with only 10.3 and 9.5% of the baseline FGF-2 remaining in these vehicles, respectively, after 2 h.

In the sugar class, only the glucose 10% w/v vehicle was able to successfully stabilise FGF-2, retaining 97.4% of the FGF-2 content after incubation at 25° C. for 2 h (FIG. 4B). Glucose was ineffective at the lower concentration of 5% w/v (residual FGF-2 content of 11.9%). Maltodextrin at 1 or 10% w/v was unable to stabilise FGF-2, the respective vehicles retaining only 24.3 and 20.5% of the baseline FGF-2 after 2 h. Similarly, mannitol at either 5 or 10% w/v was ineffective at stabilising FGF-2 solutions at 25° C., the FGF-2 content following incubation dropping to 15.4% and 19.2%, respectively. All the sugar excipients produced greater stabilisation effects on FGF-2 than water (one-way ANOVA, P<0.0001), however, only the glucose 10% w/v solution was able to successfully retain at least 90% mean residual FGF-2 content after 2 h exposure at 25° C.

The only excipient in the salt category, NaCl 0.9% w/v significantly improved the stability of FGF-2 at 25° C. compared with using water alone (Student's t-test, P<0.0001, FIG. 4A). However, NaCl 0.9% w/v failed to meet the criteria for an effective stabiliser, with just 15.5% of the baseline FGF-2 content remaining in the solution after 2 h of exposure to 25° C.

Excipients which had successfully stabilised aqueous FGF-2 solutions (≥90% baseline FGF-2 remaining) following incubation at 25° C. for 2 h were chosen as potential FGF-2 stabilisation vehicles for further evaluation and optimisation experiments. Although HPMC 0.5% w/v did not strictly meet the criteria for FGF-2 stabilisation, the mean residual FGF-2 content was statistically comparable to the MC containing solutions. On this basis HPMC 0.5% w/v was also included in the next stage of optimisation. Sodium chloride (0.9% w/v), the only salt investigated in this study, failed to stabilise aqueous FGF-2 solutions sufficiently to fit the criteria for inclusion in further studies. Therefore, only excipients from the sugar (glucose 10% w/v), polymer (MC 0.1 or 0.5% w/v and HPMC 0.5% w/v) protein (HSA 1 mg/ml) and amino acid (alanine 20 and 100 mM) classes will be represented in further studies. See FIG. 4.

FIG. 4. Effect of excipients on the stability of FGF-2 aqueous solution (770 ng/ml) incubated at 25° C. for 2 h. Samples were prepared with excipients selected from each of the four classes of protein stabilisers, (A) salts (B) sugars, (C) polymers and (D) protein/amino acids. FGF-2 content as analysed by ELISA following the incubation period is expressed as a percentage of the baseline FGF-2 content (mean±SD, n=3). Excipient descriptions have been abbreviated as follows; sodium chloride, NaCl; methylcellulose, MC; hydroxypropyl methylcellulose, HPMC and human serum albumin, HSA.

### A.3.4 Optimisation of Stabilisation Vehicles for FGF-2 Solutions

### A.3.4.1 Thermal Stability

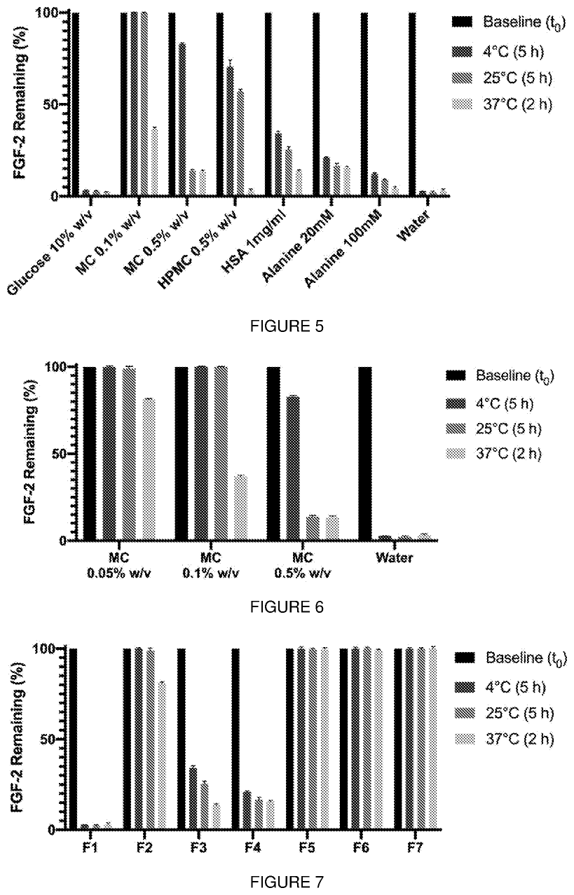

On the basis of data obtained in the survey stability study, the stabilisation of FGF-2 solutions by vehicles containing glucose (10% w/v), MC (0.1 and 0.5% w/v), HPMC (0.5% w/v), HSA (1 mg/ml) or alanine (20 and 100 mM) were evaluated in an expanded temperature stability study. The greatest stabilisation effect was observed with MC 0.1% w/v (FIG. 5). FGF-2 solutions containing MC 0.1% w/v were highly stable, retaining 100% FGF-2 content after storage for 5 h at both 4° C. and 25° C. MC 0.1% w/v also produced a superior FGF-2 stabilisation effect at 37° C. compared to the other excipients (P<0.0001), however, it was not successful at maintaining the FGF-2 content above 90% after 2 h at this temperature. No other excipient was able to effectively stabilise the FGF-2 solution at the prescribed storage conditions, although MC 0.5% w/v, HSA 1 mg/ml and alanine 20 mM were observed to produce significantly greater stabilisation effects on FGF-2 than water alone for all three storage conditions (P<0.0001). See FIG. 5.

FIG. 5. Effect of excipients on the stability of FGF-2 aqueous solutions (770 ng/ml) incubated at 4° C. for 5 h, 25° C. for 5 h and 37° C. for 2 h. Residual FGF-2 contents at baseline and following the prescribed incubation period were measured by ELISA and expressed as a percentage of the baseline concentration (mean±SD, n=3). Excipient descriptions have been abbreviated as follows; methylcellulose, MC; hydroxypropyl methylcellulose, HPMC and human serum albumin, HSA.

Comparing the excipient classes, it was observed that glucose 10% w/v was not able to effectively stabilise the FGF-2 on prolonging the incubation time from 2 h to 5 h at 25° C. Neither was it an effective stabiliser when the FGF-2 solution was incubated at 4° C. for 5 h, or at 37° C. for 2 h. The effectiveness of the polymers in stabilising FGF-2 at the prescribed conditions may be ranked in the decreasing order of MC 0.1% w/v>HPMC 0.5% w/v>MC 0.5% w/w. In the protein/amino class, HSA 1 mg/ml was a more effective FGF-2 stabiliser than alanine 20 mM for solutions stored at 4° C. or 25° C. for 5 h (P<0.0001). Alanine in turn was more effective at 20 mM than at the higher 100 mM in stabilising FGF-2 at all 3 storage conditions (P<0.0001).

Since MC exhibited concentration-dependent stabilisation effects, with the lower concentration of 0.1% w/v being more effective than the higher concentration of 0.5% w/v (FIG. 5), it was hypothesised that the stabilisation effects of MC on FGF-2 might be further improved by further decreasing the MC concentration. FIG. 6 shows that a lowering of the MC concentration to 0.05% w/v indeed resulted in improved stability of FGF-2 at 37° C. (P<0.0001). Compared with MC 0.1% and MC 0.5% w/v, which showed residual FGF-2 contents of 36.9% and 13.4%, respectively, after 2 h exposure to 37° C., MC 0.05% w/v was able to retain 81% of the baseline FGF-2 content after similar heat exposure.

FIG. 6. Effect of methylcellulose (MC) concentration on the stability of aqueous FGF-2 solutions (770 ng/ml) incubated at 4° C. for 5 h, 25° C. for 5 h and 37° C. for 2 h. Residual FGF-2 contents following the prescribed incubation period were measured by ELISA and expressed as a percentage of the baseline concentration (mean±SD, n=3).

Henceforth, the vehicles used for stabilising FGF-2 will be identified by their ID (Table 4) to facilitate discussion. The IDs are (1) water, (2) MC 0.05% w/w, (3) alanine 20 mM, (4) HSA 1 mg/ml, (5) MC 0.05% w/v with 20 mM alanine, (6) MC 0.05% w/v with 1 mg/ml HSA, and (7) MC 0.05% w/v with 20 mM alanine and 1 mg/ml HSA. The corresponding FGF-2 solutions that contained the respective stabilisers are identified by the same ID prefixed with F to denote the presence of FGF-2 in the solution (Table 5).

As no single excipient was able to stabilise FGF-2 against thermal degradation at 37° C., it was hypothesised that combinations of the excipients might provide synergistic effects. To this end, F2, being the best stabiliser vehicle based on data obtained thus far, was combined with either alanine 20 mM (F5), HSA 1 mg/ml (F6) or both alanine 20 mM and HSA 1 mg/ml (F7), and these combinations were evaluated for their stabilisation effects on FGF-2 solutions. All three combinations of excipients were found to effectively stabilise the FGF-2 solution against thermal degradation, the residual FGF-2 content in all the solutions were comparable to baseline level following the respective incubation periods of 5 h at 4° C., 5 h at 25° C. and 2 h at 37° C. (FIG. 7).

FIG. 7. Effect of excipient combinations on the stability of FGF-2 aqueous solutions (770 ng/ml) incubated at 4° C. for 5 h, 25° C. for 5 h and 37° C. for 2 h. Residual FGF-2 contents following the prescribed incubation period were measured by ELISA and expressed as a percentage of the baseline concentration (mean±SD, n=3). FGF-2 solutions have been abbreviated as follows; F1, FGF-2 in water; F2, FGF-2 and 0.05% w/v methylcellulose (MC) in water; F5, FGF-2, 0.05% w/v MC and 20 mM alanine in water; F6, FGF-2, 0.05% w/v MC and 1 mg/ml human serum albumin (HSA) in water; and F7, FGF-2, 0.05% w/v MC, 20 mM alanine and 1 mg/ml HSA in water.

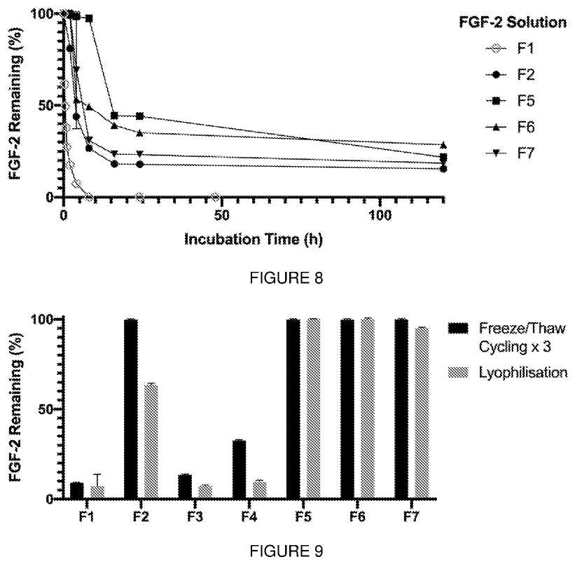

The combinations of stabilisers were further evaluated for the stabilisation of FGF-2 when stored for an extended period at 37° C. (FIG. 8). F5 (with MC and alanine as stabilisers) retained greater FGF-2 content than all other formulations at prolonged incubation of 8 h, with 97% FGF-2 remaining (P<0.0001). F6 (MC with HSA) was the second most stable, with 49.5% of FGF-2 remaining at 8 h. F7 (MC, HSA and alanine) had a similar storage stability profile to F1 (MC alone), the two solutions showing residual FGF-2 contents of 30.9% and 26.6%, respectively, after 8 h at 37° C. The rate of FGF-2 degradation slowed significantly beyond 16 h of exposure to 37° C. At 120 h of exposure to 37° C., F6 retained a greater FGF-2 content (28.8%) than all the other FGF-2 solutions tested.

FIG. 8. Effect of excipient combinations on the stability of FGF-2 aqueous solutions (770 ng/ml) stored at 37° C. for up to 5 days. Residual FGF-2 contents following the prescribed incubation period were measured by ELISA and expressed as a percentage of the baseline concentration (mean±SD, n=3). FGF-2 solutions have been abbreviated as follows; F1, FGF-2 in water; F2, FGF-2 and 0.05% w/v methylcellulose (MC) in water; F5, FGF-2, 0.05% w/v MC and 20 mM alanine in water; F6, FGF-2, 0.05% w/v MC and 1 mg/ml human serum albumin (HSA) in water; and F7, FGF-2, 0.05% w/v MC, 20 mM alanine and 1 mg/ml HSA in water.

### A.3.4.2 Processing Stability

The ability of the excipients to stabilise the FGF-2 solution against processing stressors was investigated. Solutions F1-F7 were exposed to three repeated freeze/thaw cycles or lyophilisation. The solutions were deemed stable if, after processing, the residual FGF-2 content, as measured with ELISA, did not differ from the baseline FGF-2 level by greater than 10%.

F2, F5, F6 and F7 were stable against repeated freeze/thaw cycles, with 99.8 to 100% baseline FGF-2 measured in these samples after the 3rd round of processing (FIG. 9).

FIG. 9. Effect of excipients on the stability of FGF-2 aqueous solutions (770 ng/ml) exposed to processing stressors. Solutions were frozen at −20° C. for 24 h before undergoing either three freeze/thaw cycles or lyophilisation for 24 h. Residual FGF-2 contents following the prescribed processing procedures were measured by ELISA and expressed as a percentage of the baseline concentration (mean±SD, n=3). FGF-2 solutions have been abbreviated as follows; F1, FGF-2 in water; F2, FGF-2 and 0.05% w/v methylcellulose (MC) in water; F3, FGF-2 and 20 mM alanine in water; F4, FGF-2 and human serum albumin (HSA) in water; F5, FGF-2, 0.05% w/v MC and 20 mM alanine in water; F6, FGF-2, 0.05% w/v MC and 1 mg/ml HSA in water; and F7, FGF-2, 0.05% w/v MC, 20 mM alanine and 1 mg/ml HSA in water.

Neither F3 nor F4 were effectively stabilised against repeated freeze/thaw cycles. F3 was no better than the F1 control (P=0.0929), with just 13.5% of baseline FGF-2 remaining after the 3rd round of processing. F4 was more stable (32.5% remaining) than F1 (P<0.0001), however the amount of residual FGF-2 content was well below the benchmark of at least 90% of baseline level.

Although F2 was stable to repeated freeze/thaw cycles (99.8% baseline FGF-2 remaining), there was substantial loss of functional FGF content in F2 following lyophilisation. In fact, none of the solutions containing single excipient were stable to lyophilisation, the residual FGF-2 contents of F2, F3 and F4, following lyophilisation and reconstitution with water, were 63.3%, 7.3% and 9.5%, respectively. Of these, only F2 was more stable than the F1 control (P<0.0001).

Improved results were observed when the excipients were employed in combination to stabilise the FGF-2 solution for lyophilisation. The three solutions containing excipient combinations were stable to lyophilisation (≥90% residual FGF-2 content; FIG. 9); however, F7 was slightly, though significantly, less stable than F5 and F6 (P=0.0410), suggesting that there is no advantage in using all 3 excipients in combination compared to dual combinations of MC with alanine or MC with HSA.

### A.3.5 Storage Stability of Lyophilised and Reconstituted FGF-2 Solutions

Lyophilised F5 and F6 were further studied for their storage stability, both as lyophilised dry powders and upon reconstitution of the lyophilised powders into solutions, with F1 serving as the control. The lyophilised FGF-2 powders were stored at −4° C., 4° C. and 18° C. for up to 12 months, and were deemed stable if the FGF-2 content as measured with ELISA following reconstitution of the powders with water, did not differ from baseline (FGF content in pre-lyophilised solutions) by greater than 10%. The mean baseline FGF-2 content was measured as 768, 769 and 771 ng/ml for F1, F5 and F6 solutions respectively, with the baseline FGF-2 contents not significantly different between the solutions (one-way ANOVA, P=0.2503).

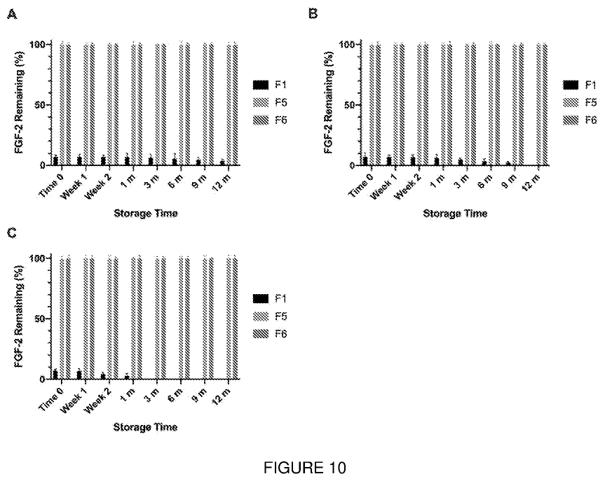

Immediately following lyophilisation, only 7.1% of the baseline FGF-2 content of F1 had remained, representing a significant drop in FGF-2 content (FIG. 10). At defined storage time points (represented by the x-axis), the FGF-2 powders were reconstituted with water and the FGF-2 content was determined by ELISA. Results are expressed as a percentage of the baseline FGF-2 content determined in the FGF-2 solutions prior to lyophilisation (mean±SD, n=3).

FIG. 10. Stability of FGF-2 aqueous solutions (770 ng/ml) upon lyophilisation and storage. FGF-2 solutions F1 (water only as vehicle), F5 (water with MC 0.05% w/v and alanine 20 mM) and F6 (water with MC 0.05% w/v and HSA 1 mg/ml) were lyophilised over 24 h then stored at −4° C. (A), 4° C. (B) or 18° C. (C) for up to 12 months. At defined storage time points (represented by the x-axis), the FGF-2 powders were reconstituted with water and the FGF-2 content was determined by ELISA. Results are expressed as a percentage of the baseline FGF-2 content determined in the FGF-2 solutions prior to lyophilisation (mean±SD, n=3).

When the lyophilised F1 powder was stored at −4° C., the FGF-2 content did not change significantly after 12 months of storage (P=0.9367). Similarly, when the storage temperature was increased to 4° C., there was no significant change in FGF-2 content over the first 9 months of storage, however, the protein was no longer detectable in the lyophilised F1 powder at 12 months of storage at 4° C. When stored at 18° C., the FGF-2 content of the lyophilised powder fell below the detectable limit by 3 months. By comparison, F5 and F6 were not only stable to lyophilisation, but the dry powders obtained were also stable to storage at −4° C., 4° C. and 18° C. for up to 12 months. The FGF-2 content of all the lyophilised F5 and F6 powders remained above 99% of baseline throughout the study period.

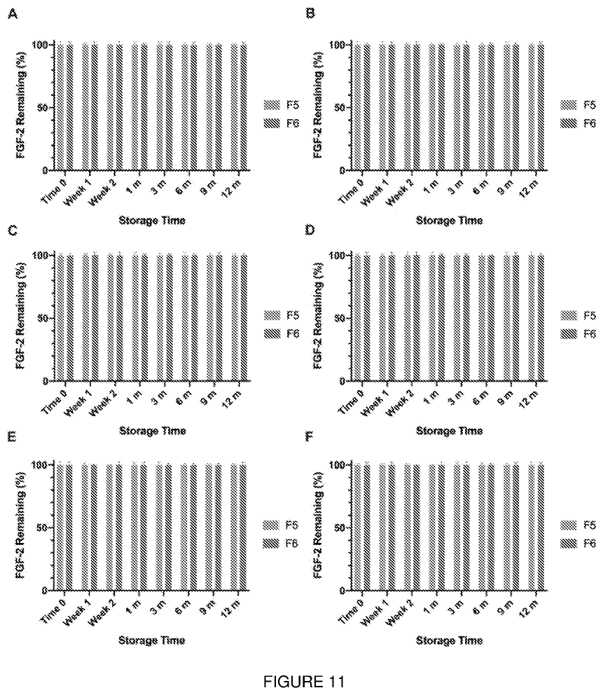

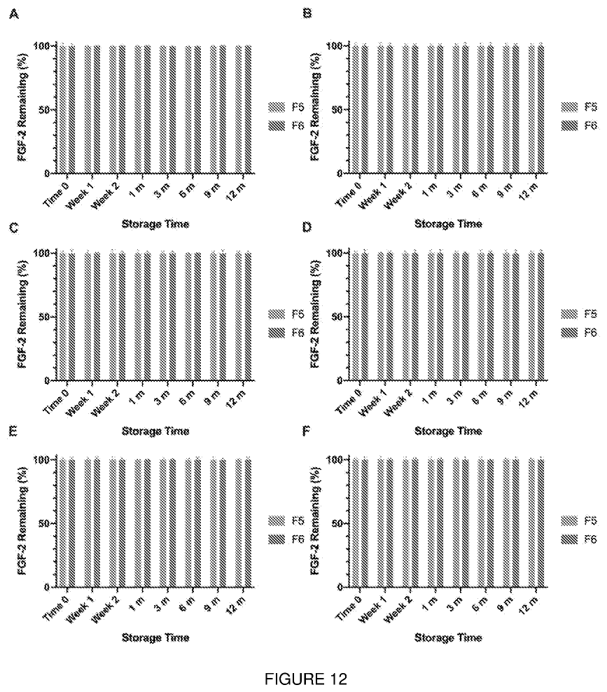

The lyophilised F5 and F6 powders following storage at −4° C., 4° C. or 18° C. for specified time points were reconstituted with water and stored at either 4° C. or 18° C. for 24 h or 7 days. The solution prepared by reconstituting the lyophilised F1 powder was not stable to storage at 4° C. or 18° C. for 24 h, with the FGF-2 content falling below the detectible limit of the ELISA assay (data not presented). By comparison, the reconstituted F5 and F6 solutions remained stable for both 24 h (FIG. 11) and 7 days (FIG. 12) at both storage temperatures, retaining >99% of the baseline FGF-2 content throughout the duration of the study.

FIG. 11. Effect of excipients on the stability of reconstituted FGF-2 aqueous solutions (770 ng/ml) over a 24 h period. FGF-2 solutions F5 (water with methylcellulose (MC) 0.05% w/v and alanine 20 mM) and F6 (water with MC 0.05% w/v and human serum albumin 1 mg/ml) were lyophilised over 24 h and the dry powders stored at −4° C. (A and B), 4° C. (C and D) or 18° C. (E and F) for up to 12 months. At defined storage time points (represented by the x-axis), the FGF-2 powders were reconstituted with water to give solutions that were then stored for 24 h at 4° C. (A, C and E) or 18° C. (B, D and F). The FGF-2 content in the stored solutions was determined by ELISA. Results are expressed as a percentage of the baseline FGF-2 content determined in the FGF-2 solutions prior to lyophilisation (mean±SD, n=3).

FIG. 12. Effect of excipients on the stability of reconstituted FGF-2 aqueous solutions (770 ng/ml) over a 7 day period. FGF-2 solutions F5 (water with methylcellulose (MC) 0.05% w/v and alanine 20 mM) and F6 (water with MC 0.05% w/v and human serum albumin 1 mg/ml) were lyophilised over 24 h and the dry powders stored at −4° C. (A and B), 4° C. (C and D) or 18° C. (E and F) for up to 12 months. At the defined storage time points (represented by the x-axis), the FGF-2 powders were reconstituted with water to give solutions that were then stored for 7 days at 4° C. (A, C and E) or 18° C. (B, D and F). The FGF-2 content in the stored solutions was determined by ELISA. Results are expressed as a percentage of the baseline FGF-2 content determined in the FGF-2 solutions prior to lyophilisation (mean±SD, n=3).

### A.4 Discussion

FGF-2 in solution may be effectively stabilised against both thermal and processing stressors in the presence of MC, with either alanine or HSA being most preferred.

The inherent instability of FGF-2 in aqueous solutions poses a major challenge for the formulation, storage and use of FGF-2 containing medicinal products. In addition to its thermal lability, the solubilised FGF-2 is highly vulnerable to aggregation and precipitation, resulting in a rapid loss of biological function. Consequently, the development of effective stabilisation strategies for FGF-2 aqueous solutions remains an area of continued research.

The lyophilised FGF-2 used in this study was recommended, by the manufacturer, to be reconstituted with water. The data in this study showed FGF-2 reconstituted in water alone (F1) underwent a rapid loss of functionality, in particular when exposed to increasing temperature. These results are consistent with published data where FGF-2 solutions have been shown to lose 50% functionality after just 46 min at 25° C., with the reported FGF-2 functional half-life decreasing to 37, 33 and 10 minutes, respectively, as the storage temperature was increased to 37° C., 42° C. and 50° C.

To render the FGF-2 sufficiently stable for processing into acceptable medicinal products that could be transported and stored without requiring −20° C. storage, a range of excipients were evaluated for their ability to stabilise FGF-2 against thermal and processing-related stressors. Excipient stabilisers were chosen from different classes, with each class expected to stabilise FGF-2 via a different mechanism.

NaCl did not stabilise FGF-2 against thermal degradation, nor did it significantly reduce the stability of FGF-2 compared with the F1 control.

Despite mannitol having been reported to successfully stabilise FGF-7 (keratinocyte growth factor), it did not protect FGF-2 against thermal degradation.

As the thermal stability study was expanded to include wider ranging temperatures and longer incubation times, it became clear that alanine was effective at stabilising FGF-2. HSA effectively stabilised FGF-2 solutions.

Lyophilisation is commonly used in the manufacture of protein products which are insufficiently stable in aqueous solutions. The inherent instability of FGF-2 was a driving factor for the development of a lyophilised FGF-2 product. It is well established that these processes often result in protein degradation through denaturation or aggregation, therefore it was important to establish that FGF-2 stabilised with the excipients identified during the thermal stability study would also be able to withstand these stressors. The FGF-2 used for this study was supplied as lyophilised powder with no additives (manufacturer's advice). Commercially, FGF-2 is commonly lyophilised in the presence of a cryoprotectant in order to preserve FGF-2 functionality. The cryoprotectant prevents structural damage and the resultant loss in functionality of the protein, often by forming hydrogen bonds with the protein as water molecules are displaced during the lyophilisation process. The results of the processing stability study support this hypothesis, with FGF-2 in water displaying a 93% loss in functional FGF-2 following lyophilisation.

Lyophilisation involves two stressors, freezing and drying, with both processes capable of damaging the protein structure. In order to be effective, the stabilisers must be effective at protecting the protein against both stressors. Many studies have shown that although a stabiliser may be effective at protecting a protein against freezing, it may not be an effective stabiliser against protein lyophilisation. In this study, although MC was effective at protecting FGF-2 against multiple freeze-thaw cycles, it was ineffective at preserving FGF-2 against lyophilisation when applied as a single stabiliser. This is likely due to its inability to protect FGF-2 against drying which often disrupts the protein structure leading to irreversible protein aggregation upon reconstitution. In addition, the successful stabilisation of a protein against lyophilisation does not always correlate with a capacity to provide storage stability. Elastase, lyophilised without any excipients was found to have retained its baseline activity immediately following lyophilisation, however 70% of its activity was lost over the subsequent 2 week storage period.

In this study, effective stabilisation of FGF-2 against lyophilisation required a combination of MC with alanine or MC with HSA. For the FGF-2 aqueous solutions, there is an advantage in combining the stabilisation effects of MC with those of alanine and/or HSA, to protect FGF-2 against lyophilisation and the subsequent storage and reconstitution of the lyophilised protein powder.

This study has clearly demonstrated the advantage of combining excipients to stabilise FGF-2 against both thermal and processing stressors. When developing a pharmaceutical product, it is desirable to use excipients in the fewest quantity and lowest concentrations possible. Therefore, due the lack of clear benefit, potential risk of increased toxicity and increased manufacturing costs associated with the use of F7, the simpler F5 and F6 formulations are preferred.

### B Example 2—In Vitro Evaluation of the Efficacy of Stabilised Basic Fibroblast Growth Factor (FGF-2) Aqeuous Solutions

### B.1 Study Aim

To demonstrate that the addition of excipient stabilisers does not negate the inherent ability of FGF-2 to promote cellular proliferation, wound healing and chemotactic migration and that the stabilised FGF solutions were superior to the non-stabilised FGF solutions in producing these biological effects.

### B.2 Materials and Methods

Experiments were conducted during an exchange program at the Guilin Medical University, China, and data were generated using primary human dermal fibroblast cultures kindly provided by Professor Jingxin Mo, Guilin Medical University.

### B.2.1 Materials

Phosphate buffered saline (PBS), penicillin/streptomycin 100X (P/S), formaldehyde 4% and propidium iodide (PI) were purchased from Beijing Solarbio Science and Technology Co. (Beijing, China). Fetal bovine serum (FBS) was purchased from Lonsera (Shanghai, China), Dulbecco's Modified Eagle Medium (DMEM) was purchased from Gibco (New York, USA) and cell counting kit-8 (CCK-8) was purchased from Dojindo Molecular Technologies (Shanghai, China). Human recombinant FGF-2 was purchased from Peprotech (Suzhou, China) and the human FGF-2 ELISA kit was purchased from Thermo Fisher Scientific (Maryland, USA). Methylcellulose USP 4000 was purchased from Professional Compounding Chemists of Australia (PCCA; NSW, Australia), and human serum albumin, DL-alanine and hydroxypropyl methylcellulose (HPMC) were purchased from Sigma-Aldrich (Missouri, USA). Deionised water was used throughout.

### B.2.2 Sample Preparation and Quantification by ELISA

A FGF-2 stock solution was prepared by reconstituting the lyophilised FGF-2 powder at 1 mg/ml (based on dry powder weight) in water. The stock solution was diluted with water to achieve a FGF-2 concentration of between 50 and 800 μg/ml and immediately assayed using a commercial ELISA kit according to the manufacturer's instructions. The absorbance at 450 nm obtained for the diluted FGF-2 solution, using a microplate reader (FilterMax F5 Multi-Mode Microplate Reader, Molecular Devices, California, USA)), was translated to FGF-2 content using a standard curve. The standard curve was plotted from the absorbance readings of standard FGF-2 solutions (15.6-1000 μg/ml) prepared according to manufacturer's instructions using the FGF-2 standard provided in the ELISA kit. The stock concentration was then adjusted to 5.2 μg/ml (active FGF-2, as determined by ELISA).

FGF-2 stabilisation vehicles 1-6 were prepared as concentrated stock solutions. The vehicle stocks were diluted with either water or the FGF-2 stock solution to give the final vehicle composition described in Table 2. The FGF-2 stock solution was similarly diluted to 1600 ng/ml (active FGF-2 as determined by ELISA) with vehicles 1-6 to prepare the stock solutions for this study. These stock solutions are identified by specific IDs described in Table 11. The stock solutions were aliquoted (10-50 μl) into 0.1 ml Eppendorf® tubes and frozen until required for further experiments. Samples for cell culture experiments were prepared by thawing the stored solutions and serially diluting with test culture medium (TCM:DMEM with 1% FBS and 1% P/S) to give the working concentrations required. Blank vehicles (Table 2) were similarly aliquoted, stored and diluted to serve as controls.

Table 6. Identification key for stabilisation vehicles and FGF-2 solutions. Solutions were prepared by combining concentrated stocks of stabilisation vehicles (1-6) with a stock FGF-2 solution (5.2 μg/ml, active FGF-2 as determined by ELISA) to give a final FGF-2 concentration of 1600 ng/ml and final excipient concentrations as described below.

| Stabi- | | Stabi- | |
|---|---|---|---|
| lisation | | lised | |
| vehicle | | Solution | |
| ID | Description of vehicle | ID | Description of Solution |
| 1 | Water (as control) | F1 | Solution of FGF-2 (1600 |
| | | | ng/ml) in vehicle 1 |
| | | | (as control) |
| 2 | Methylcellulose (MC; | F2 | Solution of FGF-2 (1600 |
| | 0.05% w/v) in water | | ng/ml) in vehicle 2 |
| 3 | Alanine (20 mM) in water | F3 | Solution of FGF-2 (1600 |
| | | | ng/ml) in vehicle 3 |
| 4 | Human Serum Albumin | F4 | Solution of FGF-2 (1600 |
| | (HSA; 1 mg/ml) in water | | ng/ml) in vehicle 4 |
| 5 | MC (0.05% w/v) and | F5 | Solution of FGF-2 (1600 |
| | alanine (20 mM) in water | | ng/ml) in vehicle 5 |
| 6 | MC (0.05% w/v) and HSA | F6 | Solution of FGF-2 (1600 |
| | (1 mg/ml) in water | | ng/ml) in vehicle 6 |

### B.2.3 Cell Culture

Primary human dermal fibroblasts isolated from healthy human adult skin were seeded at a density of approximately 2.2×10⁶ cells in 10 ml of complete culture media (CCM:DMEM with 10% FBS and 1% P/S) in a 100 mm culture dish (Eppendorf, Hamburg, Germany) and cultured until confluence in preparation for future studies. Cell cultures were incubated at 37° C. under an atmosphere of 5% CO2 (Forma Series II Water Jacket CO2 Incubator, Thermo Scientific, Massachusetts, USA) unless otherwise stated.

### B.2.4 Dose-Response Assay

Fibroblasts (5×103 cells in 100 μl of CCM) were added to each well of 96-well culture plates (Corning, New York, USA). After incubation for 24 h, the CCM was replaced with test culture media (TCM:DMEM with 1% FBS and 1% P/S) and the cells cultured for a further 24 h to arrest cell growth. FGF-2 solutions and blank vehicles were applied in escalating doses (equivalent to 0.0098-200 ng/ml FGF-2) to the fibroblasts in triplicate and cultured for 48 h. The samples were removed and replaced with 100 μl of CCK-8 which had been diluted 1:10 with TCM. The plates were then incubated for 1 h before the absorbance in each well was measured at 450 nm with a plate reader. The dose-response profile was determined by subtracting the absorbance values of the corresponding vehicle samples from the absorbance values of the FGF-2 samples, and plotting the net value against the FGF-2 dose with a 4 parameter logistic fit (GraphPad Prism 8, California, USA) to calculate the half maximal effective concentration (EC50) value for each of the solutions.

### B.2.5 Wound Healing Assay

The cellular regenerative capacity of the FGF-2 solutions was investigated via a wound healing scratch assay. Fibroblasts were seeded at a density of 2×104 cells/well with 1 ml CCM in 24-well culture plates (Corning, New York, USA) and cultured for 48 h to reach confluency. The CCM was replaced with TCM and the cells cultured for a further 24 h to achieve growth arrest. A scratch was made in the confluent cell monolayer with a plastic disposable pipette tip (200 μl) and cultures were washed twice with PBS to remove non-adherent cells. The cells were then incubated with 1 ml FGF-2 solutions (50 ng/ml, prepared by diluting FGF-2 stock solutions F1-6 with TCM) or stabilisation vehicles (1-6, similarly diluted with TCM) for 24 h. Images of the cell cultures were taken (Microscope: Nikon Eclipse Ti-S, Camera: Nikon DS-Ri2, software: NIS Elements v5.01, Nikon Corporation, Tokyo, Japan) at 0, 8 and 24 h. The images were processed using ImageJ (National Institutes of Health, Maryland, USA) and the wound area was determined by using an algorithm which distinguishes cell free areas from cell populated areas based on differences in local texture homogeneity. The percentage wound closure was calculated by subtracting the remaining wound area at 8 or 24 h from the baseline wound area at 0 h, and expressing the result as a percentage of the baseline wound area. The experiment was performed in triplicates and percentage wound closure presented as mean±SD.

### B.2.6 Chemotactic Migration Assay

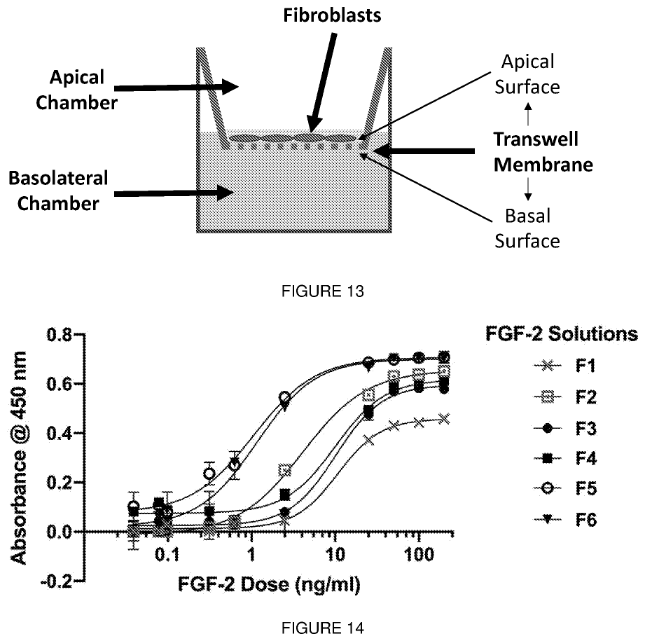

Fibroblast chemotactic migration was assessed using the Boyden well chamber technique. Fibroblasts were seeded at 5×104 cells suspended in 200 μl of TCM into the apical chamber of 24-transwell culture plates (8 μm pore size; Corning, New York, USA) and incubated overnight with 500 μl of TCM in the basolateral chamber to allow the cells to attach. FGF-2 solutions (F1-6; 50 ng/ml) and the corresponding stabilisation vehicle samples (1-6) were added to replace the TCM in either the basolateral chamber only (500 μl), or both the apical and basolateral chambers (200 μl and 500 μl, respectively; FIG. 13). Fibroblasts were seeded onto the apical surface of the transwell membrane with the addition of FGF-2 solutions (F1-6) or stabilisation vehicles (1-6) to either the basolateral only, or both apical and basolateral chambers, and the chemotactic migration of the cells measured by the number of cells present on the basal surface of the transwell membrane at 24 h.

The addition of samples to only the basolateral chamber created a concentration gradient between the apical and basolateral chambers allowing chemotactic migration to be detected. In contrast, the addition of samples to both the basolateral and apical chambers resulted in no concentration gradient between the chambers and controlled for chemokinetic migration. Plates were cultured for a further 24 h, and the cells that remained on the apical surface of the membrane (non-migratory) were removed with a cotton swab soaked in PBS so that only those cells that had migrated and attached to the basal surface of the membrane would be counted. The transwells were washed in PBS to remove non-adherent cells and residual culture media before they were soaked in 4% formaldehyde for 30 min to fix the cells. The cells were washed with PBS, stained with 10 M propidium iodide for 20 min, and visualised by fluorescence microscopy (excitation 535 nm, emission 615 nm; Leica DM4 B; Leica Microsystems, Wetzlar, Germany). Photographs were taken (Leica DFC7000T camera, Leica Microsystems, Wetzlar, Germany) and processed (Leica Application Suite X, Leica Microsystems, Wetzlar, Germany).

The photographic images were analysed using ImageJ to determine the number of cells which had migrated to the basal surface of the transwell membrane. Seven images per membrane were obtained to ensure coverage of the entire surface of the basal membrane. The cell count from each image was determined with the sum of these cell counts for the seven images representing the cells which had migrated for that transwell. The experiment was performed in triplicate and the cell count presented as mean±SD.

### B.2.7 Data Analysis

Results are expressed as mean±SD. Data were analysed by two-way ANOVA with post-hoc Tukey's test (GraphPad Prism 8, California, USA) applied for paired comparison of means, unless stated otherwise. A P value ≤0.05 was considered to be significant.

### B.3 Results

### B.3.1 Dose-Response Assay

FIG. 14. Cellular proliferation curves of primary human dermal fibroblasts in response to escalating doses (0.0098-200 ng/ml) of FGF-2 aqueous solutions containing different stabilisers. FGF-2 solutions F1 (water only as vehicle), F2 (water with methylcellulose (MC) 0.05% w/v), F3 (water with alanine 20 mM), F4 (water with human serum albumin (HSA) 1 mg/ml), F5 (water with MC 0.05% w/v and alanine 20 mM) and F6 (water with MC 0.05% w/v and HSA 1 mg/ml) were applied to fibroblasts at escalating doses. Cytoproliferative effects were measured via a CCK-8 assay and the net absorbance (absorbance of culture wells for FGF-solution minus absorbance of culture well for corresponding vehicle) calculated. Each point represents the mean±SD (n=3).

The addition of escalating doses of stabilised FGF-2 solutions (F2-F6) to the fibroblasts resulted in varying proliferative effects (FIG. 14). At the lowest applied FGF-2 dose of 0.0098 ng/ml, none of the stabilised FGF-2 solutions showed significant differences in cytoproliferative effects compared to the F1 control (one-way ANOVA, P=0.1252). As the FGF-2 dose was increased, differences in cytoproliferative effects between the solutions became more apparent and, while all solutions showed a plateauing of activity at a FGF-2 dose ≥50 ng/ml, there were differences in maximum activity exhibited by the solutions. At high doses of 50-200 ng/ml, F5 and F6 produced comparable cytoproliferative effects (P=0.8399) that were stronger than the activities of the other FGF-2 solutions. Of the two, F5 was more effective at lower concentrations, its cytoproliferative effect was consistently higher than the other solutions, including F6, over a broader concentration range (0.078-2.5 ng/ml). By comparison, F2 was as effective as F6 at low doses between 0.0098 and 2.5 ng/ml (P=0.4073) but was less effective than both F5 and F6 at higher doses. In a similar manner, F4 at low doses (0.039-0.078 ng/ml) produced comparable cytoproliferative effects as F5 that were greater than the activities of the other FGF solutions, however, it was inferior to F5, F6 and F2 at doses higher than 5 ng/ml. By contrast, F3 showed comparable activity to F4 at high doses (≥50 ng/ml) but was no different in activity to the control solution at low FGF doses. At FGF-2 doses greater than 25 ng/ml, all stabilised FGF-2 solutions produced greater proliferative effects in the fibroblasts than the control (F1, P<0.0001). On this basis, the cytoproliferative activity of the FGF-2 solutions on the primary human dermal fibroblasts may be ranked in the following decreasing order: F5>F6>F2>F4>F3>F1. EC50 values determined from the dose-response curve generally support the ranking, with the control (F1) displaying the greatest EC50 (10.754 ng/ml), followed by comparable values for F3 (10.191 ng/ml) and F4 (10.17 ng/ml), then F2 (4.104 ng/ml), F6 (1.145 ng/ml) and F5 (1.064 ng/ml).

FGF-2 doses greater than 50 ng/ml were not associated with significant increases in cytoproliferative activity regardless of stabilisation vehicle. Given the small differences in cellular proliferative effects between FGF-2 doses of 50 and 200 ng/ml, adequate differentiation between the FGF-2 solutions and constraints in budget and FGF-2 availability, 50 ng/ml was chosen as the threshold FGF-2 dose for maximal cellular activity and was carried through to all further studies in this section. As such, from this point onwards FGF-2 solutions F1-F6 will refer to the FGF-2 stock solutions (Table 2) diluted to a final concentration of 50 ng/ml FGF-2 with TCM.

### B.3.2 Wound Healing Assay

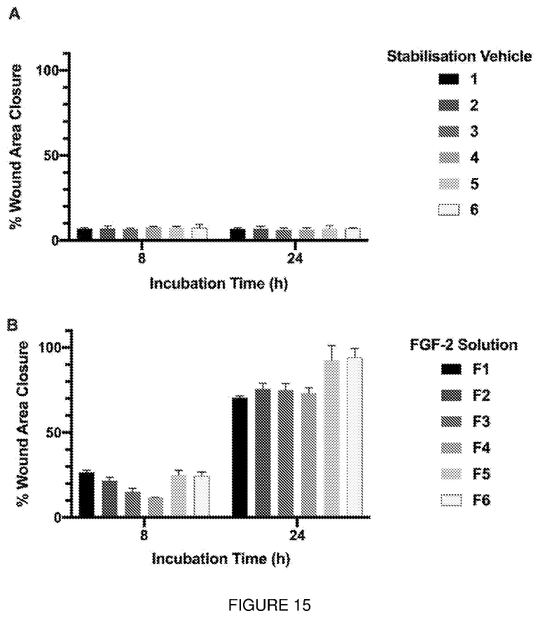

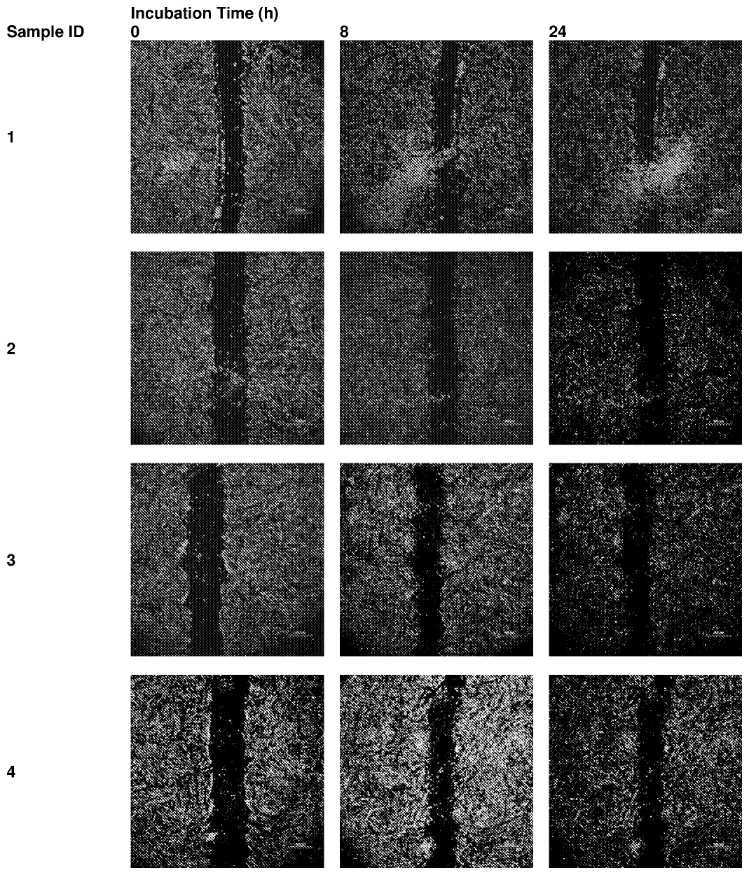

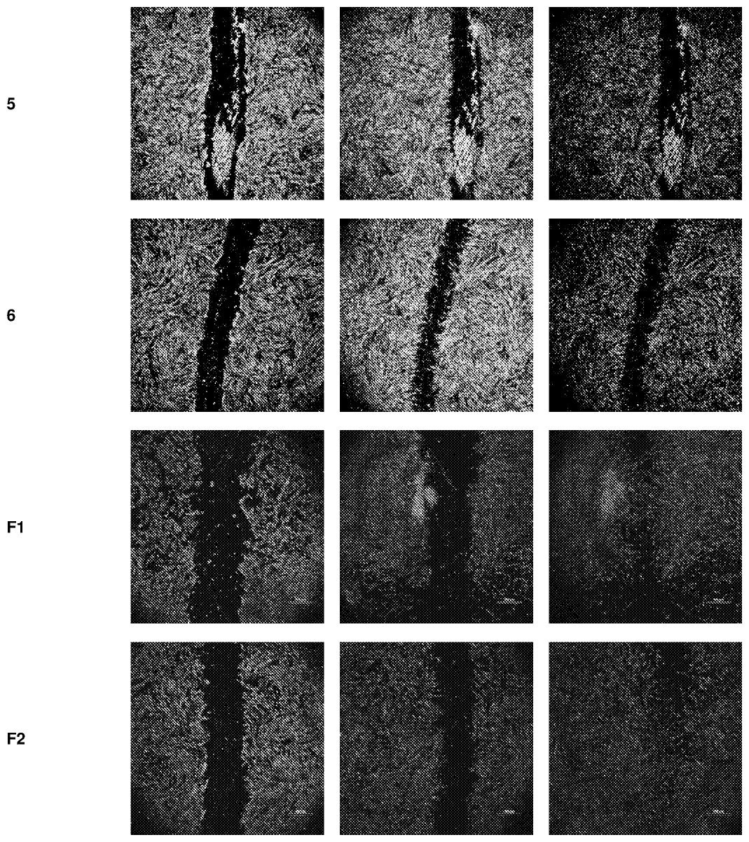

FIG. 15. Wound healing capacity of stabilized FGF-2 solutions. Fibroblasts were grown to confluency before a wound was created in the cell monolayer by drawing a pipette tip across the base of each well in a single line. Cells migrated to cover the cell-free, simulated wound area following exposure to either stabilisation vehicles (A) or FGF-2 solutions (B). Each solution was identified by the vehicle composition; vehicle 1 (water only), vehicle 2 (water with methylcellulose (MC) 0.05% w/v), vehicle 3 (water with alanine 20 mM), vehicle 4 (water with human serum albumin (HSA) 1 mg/ml), vehicle 5 (water with MC 0.05% w/v and alanine 20 mM) and vehicle 6 (water with MC 0.05% w/v and HSA 1 mg/ml). FGF-2 solutions (F1-F6) contained 50 ng/ml FGF-2 in the corresponding vehicles. Closure of the inflicted wound area was determined as a percent relative to the baseline wound area after 8 or 24 h of exposure, with each data point representing the mean±SD (n=3).

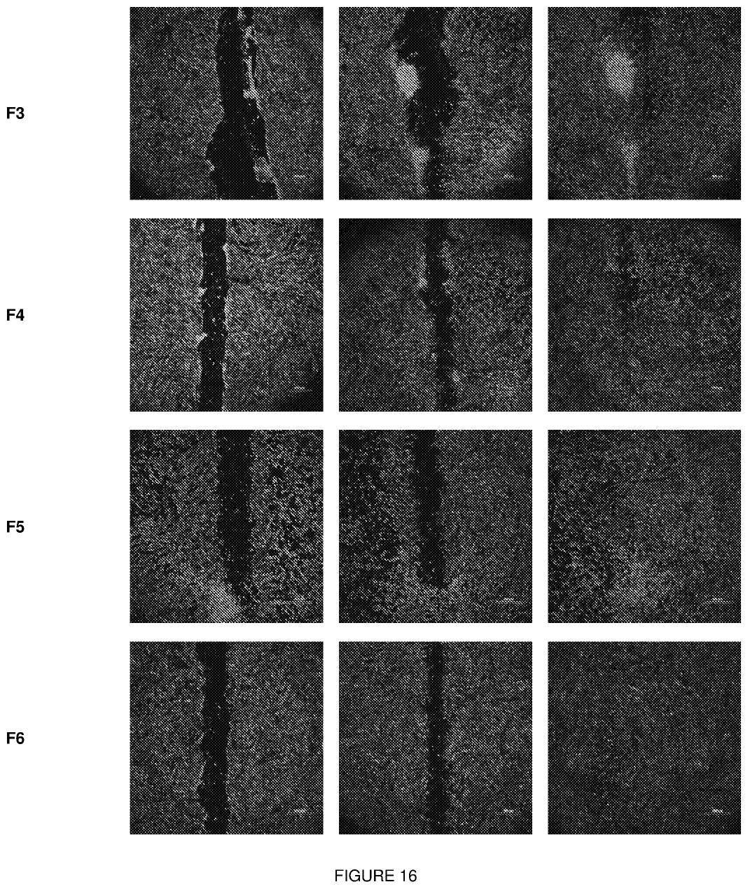

A simulated wound of the primary dermal fibroblast monolayer was exposed to each of the stabilisation vehicles (1-6) and FGF-2 solutions (F1-F6, containing 50 ng/ml FGF-2) to assess the wound healing capacity of FGF-2 (FIG. 15). All vehicles stimulated only minimal wound closure (5-9%) over the 24 h period, with no significant differences in wound closure observed amongst the solutions (P=0.9533). Representative optical microscope images of simulated wounds are presented in FIG. 16.

FIG. 16. Representative optical micrographs of simulated wounds in a human dermal fibroblast monolayer exposed to blank vehicles and FGF-2 solutions. Wounds were created by drawing a pipette tip across the base of each well in a single line, disturbing the fibroblast monolayer. Cells migrated to cover the cell-free, simulated wound area following exposure to either stabilisation vehicles (sample 1-6) or FGF-2 solutions (sample F1-F6). Each sample was identified by the vehicle composition; vehicle 1 (water only), vehicle 2 (water with methylcellulose (MC) 0.05% w/v), vehicle 3 (water with alanine 20 mM), vehicle 4 (water with human serum albumin (HSA) 1 mg/ml), vehicle 5 (water with MC 0.05% w/v and alanine 20 mM) and vehicle 6 (water with MC 0.05% w/v and HSA 1 mg/ml). FGF-2 solutions (F1-6) additionally contained 50 ng/ml FGF-2. Images were taken at 0, 8 and 24 h after exposure to samples. All images were captured at 100× magnification, scale bar=500 μm.

By comparison, exposure of the wound to FGF-2 solutions resulted in greater wound area closure at both 8 and 24 h. At 8 h post exposure, F1, F2, F5 and F6 displayed comparable wound closure activities that were significantly greater than the wound closures seen with the F3 (F1: P=0.0121; F2: P=0.0482; F5: P=0.0413 and F6: P=0.0473) and F4 samples (F1: P=0.007; F2: P=0.0492; F5: P=0.0024 and F6: P=0.0046). F1, however, was no different to F2, F3 and F4 at 24 h of exposure (70.5, 75.7, 74.9 and 73.2% respectively), whereas F5 and F6 continued to produce significantly greater wound healing than all other solutions (P<0.0001) with the wound areas reduced by 92.5% and 94.1%, respectively, at 24 h.

### B.3.3 Chemotactic Migration Assay

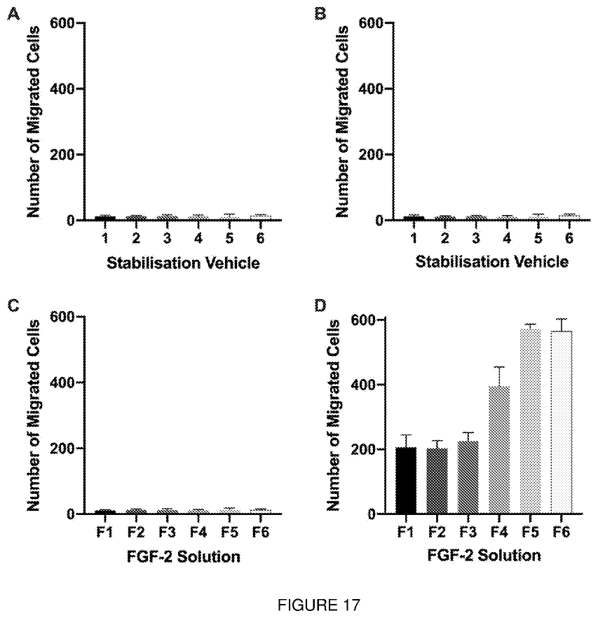

Chemotactic migration of fibroblasts was studied by creating a concentration gradient of FGF-2 in a transwell set-up, and employing stabilisation vehicles for control experiments. None of the transwells with stabilisation vehicles added to only the lower basolateral chamber, or to both the upper apical and lower basolateral chambers of the transwells, resulted in >20 cells migrating from the apical to the basal surface of the membrane (FIG. 17).

FIG. 17. Comparison of the number of human dermal fibroblasts which underwent chemotactic migration following 24 h exposure to stabilisation vehicles (1-6) in both the upper and lower chambers (A), or lower chamber only (B); or FGF-2 solutions (F1-F6) in both the upper and lower chambers (C), or lower chamber only (D) of a transwell set-up.

Each sample was identified by the vehicle composition; vehicle 1 (water only), vehicle 2 (water with methylcellulose (MC) 0.05% w/v), vehicle 3 (water with alanine 20 mM), vehicle 4 (water with human serum albumin (HSA) 1 mg/ml), vehicle 5 (water with MC 0.05% w/v and alanine 20 mM) and vehicle 6 (water with MC 0.05% w/v and HSA 1 mg/ml). FGF-2 solutions (F1-F6) additionally contained 50 ng/ml FGF-2. Results are expressed as the mean number of migrated cells/well ±SD (n=3).

The addition of FGF-2 (50 ng/ml) containing solutions to both the upper and lower chambers of the transwell also did not result in a significantly different number of cells migrating to the basal surface of the membrane when compared to data obtained with the corresponding stabilisation vehicle alone (P=0.8265).

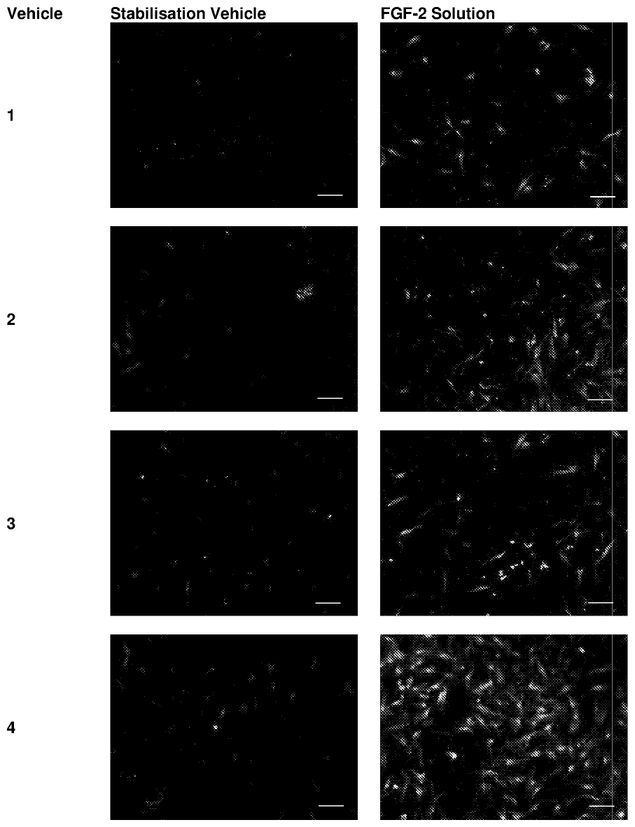

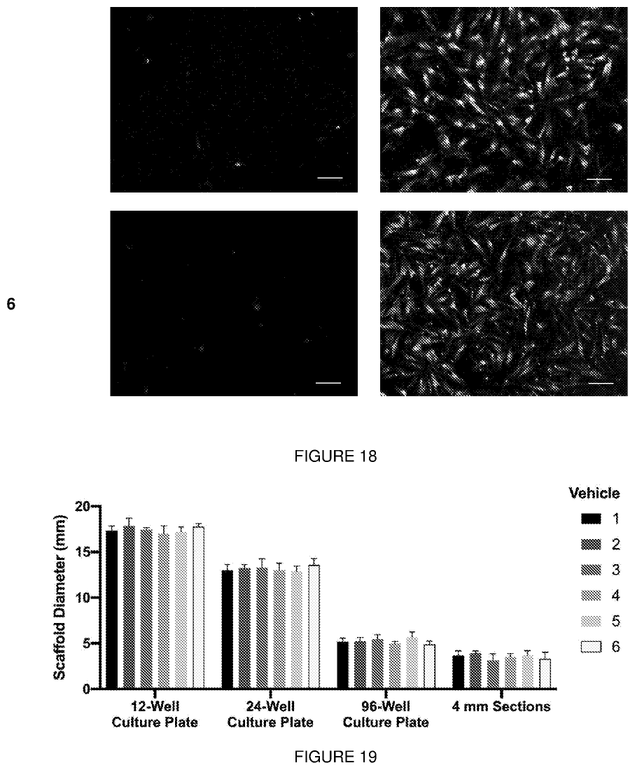

In contrast, the addition of FGF-2 solutions to only the lower chamber resulted in high cellular migration to the basal surface of the transwell membrane (FIG. 18), the number of migrated cells increased by 10- to 30-fold compared with the corresponding vehicle (P<0.0001).

FIG. 18. Representative fluorescence micrographs of human dermal fibroblast cells which had undergone chemotactic migration to the basal surface of a transwell membrane in response to FGF-2. Cells seeded on the apical surface of a transwell membrane were exposed to FGF-2 solutions or the corresponding stabilisation vehicles added in the basolateral chamber of a transwell set-up. Each sample was identified by the vehicle composition; vehicle 1 (water only), vehicle 2 (water with methylcellulose (MC) 0.05% w/v), vehicle 3 (water with alanine 20 mM), vehicle 4 (water with human serum albumin (HSA) 1 mg/ml), vehicle 5 (water with MC 0.05% w/v and alanine 20 mM) and vehicle 6 (water with MC 0.05% w/v and HSA 1 mg/ml). FGF-2 solutions additionally contained 50 ng/ml FGF-2. All images were captured at 200× magnification, scale bar=1 mm.

F5 and F6 produced comparable chemoattractant effects that were strongest amongst the FGF-2 solutions, while F1, F2 and F3 produced comparable lowest effects. F4 produced intermediate chemoattractive effects when compared to the other FGF solutions. The ranking order of chemoattractive potential is: F5=F6>F4>F1=F2=F3.

### B.4 Discussion

Stabilisation of FGF-2 potentiates the efficacy of FGF-2 in an in vitro model, with FGF-2 stabilised by the addition of MC and either alanine or HAS being preferred.

FGF-2 is known to exert proliferative, migratory and chemoattractive effects in a variety of tissues. These properties make FGF-2 an attractive component for wound healing and tissue engineering constructs. However, the rapid degradation of FGF-2 in aqueous solution has significantly hampered the development of FGF-2 containing pharmaceutical products. It was hypothesised that the stabilisation of FGF-2 in aqueous solutions, would enhance the cytoproliferative, cellular migratory and chemoattractant effects of FGF-2. This study set out to confirm these effects by generating in vitro cell-based data via dose-response, wound healing and chemotactic migration assays respectively.

It is well documented in the literature, that FGF-2 dispersed in water is not thermally stable and is quickly inactivated, with a half-life of 37 min when exposed to temperatures. Therefore, when the control (F1) FGF-2 solutions were added to the cell cultures, as in this study, it was expected that the FGF-2 would be rapidly inactivated, and apparent cellular proliferative effects could only be observed when the FGF-2 dose was sufficiently high. This hypothesis was confirmed by the dose-response assay, with FGF-2 in water (F1) having the highest EC50 value (10.754 ng/ml) and the lowest maximal proliferative effect of all the FGF-2 containing solutions used in the study. On the basis of the stability results from a previous study, Example 1, it was also expected that the F5 and F6 solutions would have the greatest stability at 37° C. and to then demonstrate the greatest cytoproliferative responses on the fibroblasts. Indeed, the F5 and F6 solutions produced maximal proliferative responses in the human dermal fibroblasts that were approximately 10-fold higher than that of F1, and their EC50 values (1.064 and 1.145 respectively) were the lowest of the FGF-2 solutions. These effects of F5 and F6 are similar to the 10-fold lowering in EC50 value observed following the addition of the endogenous stabiliser, heparin to an FGF-2 solution, suggesting that the enhanced cytoproliferative effects of F5 and F6 are a direct influence of the combination of stabilisers present in these solutions.

The density of FGF-2 receptors and subsequent responsiveness of various cells and tissues to external FGF-2 stimuli, as well as the purity of the FGF-2 sample, are likely to dictate the optimal FGF-2 dose for a particular clinical application. As a result, a threshold FGF-2 dose had to be established for this study for the human dermal fibroblasts. It is noted that maximal proliferative effects of unstabilised FGF-2 on fetal bovine heart epithelial cells occurred at doses of about 100 ng/ml, which is double the threshold dose of 50 ng/ml identified in this study.

Although FGF-2 is able to exert a biological effect very rapidly, it is quickly inactivated both in vitro and in vivo due to protein aggregation and degradation. Therefore, the early wound healing response observed at 8 h following exposure to solution F1 was most likely unable to be maintained over the study period due to the rapid inactivation of FGF-2. In contrast, the wound healing responses observed at 8 h following exposure to solutions F5 and F6 were successfully maintained over the study period of 24 h, that then led to greater wound area closure. This indicated that the FGF-2 in solutions F5 and F6 was sufficiently stabilised to allow FGF-2 to exert its biological effects over a more prolonged period than F1.

It was not expected that the blank stabilisation vehicles would promote wound healing. In this study, the addition of blank vehicles to the human dermal fibroblast cultures only resulted in minimal wound healing, and, over the course of 24 h, many samples began to exhibit lower cell counts that are indicative of cell distress, likely due to the lack of nutrients in the vehicle and TCM media. In addition, the stabilisers themselves had no significant impact on cellular migration. Therefore, the results obtained for the FGF-2 containing solutions could be directly attributable to the presence and relative stability of FGF-2 in the solutions.

The main limitation of a scratch wound assay is that it is unable to differentiate between chemotactic and chemokinetic cellular migration in response to FGF-2. The differentiation between chemotactic and chemokinetic cellular migration was achievable via the transwell system, where an FGF-2 concentration gradient could be established across the apical and basolateral chambers. Through the addition of test samples to either only the basolateral chamber of the transwell set-up (creation of concentration gradient between apical and basolateral) or to both the apical and basolateral chambers (no concentration gradient between apical and basolateral), it is possible to distinguish between chemotactic and chemokinetic migration. The addition of blank vehicles (1-6) to either the basolateral chamber only or both the apical and basolateral chambers of the transwell resulted in no differences in the number of cells migrating from the apical to basal surface of the transwell membrane. This indicated that the stabilisation vehicle alone did not promote cellular migration. Conversely, the addition of FGF-2 containing solutions to only the basolateral chamber of the transwell set-up resulted in significantly greater cellular migration to the basal surface of the transwell membrane than was observed when the FGF-2 containing solutions were added to both the apical and basolateral chambers of the system, indicating that indeed chemotaxis rather than chemokinesis was involved. As non-migrated cells were removed from the apical surface of the transwell membrane prior to imaging, it is likely that only a sub-population of the cell culture participated in the migration towards FGF-2 within the study period. It is unclear whether this is due to a time factor or differential levels of expression of FGF-2 receptors on the cells.

The composition of FGF-2 solution played a significant role in the degree of cellular migration observed. F4 displayed stronger chemoattractive effects than F1, F2 and F3, which could be attributed to the presence of HSA in F4. The human dermal fibroblasts were ‘starved’ through the replacement of CCM with TCM prior to exposure to FGF-2 solutions. HSA or, more commonly, BSA are routinely added to cell culture media to optimise cellular growth. Therefore, the HSA in F4 may have stimulated the migration of starved cells towards the nutrients, resulting in a greater migratory effect than observed with F1, F2 and F3. HSA was also present in F6, but not F5, yet both showed comparable chemotactic effects that were stronger than F4. This could be attributed to the increased stability of FGF-2 in F5 and F6, which would allow the FGF-2 activity to be sustained at a higher concentration over a longer period of time. In turn, this might allow F5 and F6 to show enhanced proliferative, migratory and chemoattractive effects on the human dermal fibroblasts when compared to all other FGF-2 solutions.

The results of this study suggest that the stabilisation of FGF-2 potentiates the endogenous effects of FGF-2 in an in vitro model.

### C Example 3: Development and Characterisation of an Optimised Alginate-Based Scaffold Material

### C.1 Study Aim

To determine the conditions for the preparation of alginate-based scaffold materials, that would provide optimal properties for the loading of FGF-2, for the treatment of chronic TM perforations.

### C.2 Materials and Methods

### C.2.1 Materials

Calcium chloride (CaCl2) was purchased from Chem-Supply Pty Ltd. (South Australia, Australia).

### C.2.2 Preparation of Prototype Scaffold Materials

Calcium alginate, obtained by crosslinking sodium alginate with calcium ions, was chosen as the prototype scaffold material on the basis of its GRAS status, previous use in wound healing applications, evidence of biocompatibility and the relatively simple/easily customisable methodology required to produce calcium alginate scaffolds. For comparison, the commercially available gelatin-based scaffold material, Gelfoam® (Pfizer, New York, USA), was also evaluated.

### C.2.2.1 Preparation of Blank Scaffold Materials

Low-viscosity sodium alginate was dissolved in water to produce 1.5, 2 or 3% w/v solutions and these were added in various volumes into each well of a 12-, 24-, or 96-well culture plate (Corning, New York, USA) to obtain scaffolds of different diameters and thickness. The liquid-filled plates were frozen at −20° C. (Westinghouse Freezer FJ302V-L, Westinghouse Electric Corporation, Pennsylvania, USA) for 16 h before an equal volume of CaCl2 solution (25, 50 or 100 mM) was added to each well and allowed to crosslink with the thawing sodium alginate solution for 20 min at ambient temperature. The addition of CaCl2 to the frozen sodium alginate solution allowed the crosslinkage reaction to occur in a controlled way over the entire surface, penetrating in depth as the alginate solution thawed, and producing a more uniformly crosslinked scaffold material. Excess CaCl2 was removed by completely filling each well with water, allowing the scaffold to soak for 2 min, then removing all liquid from the well. The washing process was repeated three times before water was added to each well to completely immerse the scaffold and the soaked scaffolds were frozen at −20° C. for 16 h prior to lyophilisation (Alpha 1-2 LDplus, Martin Christ Gefriertrocknungsanlagen GmbH, Osterode am Harz, Germany) for 24 h.

Table 7. Volume of alginate used to prepare prototype alginate-based scaffold materials for scaffold optimisation experiments. Volumes were dependent upon the mould used to prepare the scaffold material and classified as either low, medium or high for each mould size based on the expected thickness of the resultant scaffold. On the basis of the mould diameter, each of the low, medium and high volumes were expected to produce scaffolds with a thickness of approximately 1.5, 3.0 and 5.0 mm, respectively.

| | Sodium alginate solution volume (ml) | | |
|---|---|---|---|
| | Mould Description | | |
| Volume | 12-Well | 24-Well | 96-Well |
| Classification | Culture Plate | Culture Plate | Culture Plate |
| Low | 0.5 | 0.3 | 0.05 |
| Medium | 1 | 0.5 | 0.1 |
| High | 2 | 1 | 0.15 |

TABLE 8
Properties of culture plates used as moulds for
alginate scaffold production. Details have been
compiled from manufacturer information.

| | Mould | Maximum | | |
|---|---|---|---|---|
| | Diameter | Volume | | |
| Mould | (mm) | (mL) | Material | Manufacturer |
| 12-Well Culture | 22.1 | 6.9 | Polystyrene | Corning |
| Plate (M12) | | | | |
| 24-Well Culture | 15.6 | 3.4 | Polystyrene | Corning |
| Plate (M24) | | | | |
| 96-Well Culture | 6.35 | 0.36 | Polystyrene | Corning |
| Plate (M96) | | | | |

### C.2.2.2 Preparation of Blank Scaffold Materials Using Different Vehicles to Dissolve the Sodium Alginate

Following the characterisation experiments on scaffolds prepared, in which water was used as the vehicle, a medium volume of 2% w/v sodium alginate, crosslinked with 50 mM CaCl2 was identified as the optimal combination for preparing the alginate scaffolds, irrespective of mould size. To determine whether the substitution of water with the vehicles used to evaluate FGF-2 stability would affect the physical characteristics of the alginate scaffold, scaffolds were prepared except that the alginate solution was prepared using each of the stabilisation vehicles in place of water as dissolution medium.

### C.2.2.3 Preparation of FGF-2 Loaded Scaffold Materials

To determine whether the addition of FGF-2 would affect the physical characteristics of the alginate scaffold, the scaffolds were prepared with 2% w/v sodium alginate solutions containing 10.5 μg/ml of FGF-2 and using the stabilisation vehicles as dissolution media. Sodium alginate was dissolved in each of the stabilisation vehicles (1-6), and made up to 90% of the final volume required to produce a 2% w/v sodium alginate solution. The remaining 10% of the volume was comprised of FGF-2 in the corresponding stabilisation vehicle (1-6). An FGF-2 stock solution (185 μg/ml in water, FGF-2 content confirmed by ELISA) was diluted with the stabilisation vehicles (1-6) to prepare 105 μg/ml solutions, and these were added (100 μl/ml) to the corresponding sodium alginate solutions to give a final FGF-2 concentration of 10.5 μg/ml. The solutions were vortexed for 2 min to ensure homogeneity, before 100 μl of each solution (containing 1050 ng FGF-2) was added to the wells of a 96-well culture plate (Corning, New York, USA). Due to the prohibitive cost of FGF-2, only a single scaffold size was prepared, using the 96-well plate and a medium volume of alginate solution crosslinked with 50 mM CaCl2.

### C.2.3 Characterisation of Scaffold Materials

The physical properties of alginate scaffolds were investigated in order to determine the optimal volume of sodium alginate and combination of sodium alginate and CaCl2 concentrations required to produce a consistent product with desirable properties for clinical use. An ideal scaffold would have consistent dimensions and weight, maintain its structural integrity during transport and handling, and hydrate at a controlled rate so as to release its loaded cargo over a period of at least two weeks. Additionally, the scaffold material would be porous with an appropriate architecture to promote tissue regeneration.

Not all chronic TM perforations are the same size or shape. As such, it is expected that the scaffold material may require cutting or shaping for individualised treatment in a clinical setting. Therefore, the largest scaffolds prepared using the 12-well culture plate were additionally cut into discs using a 4 mm biopsy punch to determine whether the physical properties of the scaffold would change upon cutting. The ideal scaffold material would retain its physical characteristics even when cut. The 4 mm biopsy punch was chosen for this purpose on the recommendation of Prof. Gunesh Rajan as, in his surgical experience, this instrument is readily available and commonly used in the clinical setting.

The Gelfoam® sponge has been successfully used in combination with FGF-2 to promote the healing of chronic TM perforation in clinical trials. The Gelfoam® sponge was therefore used as a comparator for the evaluation of the alginate scaffold materials. To facilitate discussion in this section, the alginate scaffolds will be referred to as test scaffolds and Gelfoam® scaffolds will be referred to as control scaffolds.

### C.2.3.1 Diameter

The diameter of the test scaffolds were measured to determine whether the method of preparation was able to produce consistent and uniformly sized scaffold materials. Test scaffolds were removed from the moulds and placed on a ruler with 0.5 mm markings. Test and control scaffolds were additionally cut into discs using a 4 mm biopsy punch (Kai Medical, Gifu, Japan). The diameter of these sections were measured in the same manner as the full-size scaffolds.

### C.2.3.2 Thickness

The thickness of the test scaffold materials were measured to determine whether the method of preparation was able to produce scaffold materials of uniform thickness. A tablet caliper (Mitutoyo 543-783B Absolute, Mitutoyo Corporation, Kanagawa, Japan) was used to measure the scaffold thickness. The distance between the arm and the base plate of the caliper was tared in the neutral position prior to scaffold measurement. The thickness of test scaffolds was measured in mm after removal from the moulds, and after they were cut using the biopsy punch. The thickness of the control scaffolds, as supplied and after cutting using the biopsy punch, were also measured for comparison. Test scaffolds prepared in the 12- and 24-well plates had diameters larger than the caliper, and their thickness was measured at 5 different positions. Similarly, due to the large size of the Gelfoam® scaffold (as supplied), the thickness of this scaffold was measured at 9 different positions. The mean value of these measurements was taken as the thickness of the scaffold.

### C.2.3.3 Weight

The mean weight (mg) of the test and control scaffold materials (n=6) were measured (Pioneer®) analytical balance, Ohaus Corporation, New Jersey, USA) to determine whether the method of preparation was able to produce scaffold materials of uniform weight.

### C.2.3.4 Friability

In order to be clinically useful, the scaffolds must retain their structural integrity during transport and during insertion into a TM perforation. The effect of transport and handling on scaffold integrity was examined by measuring scaffold friability, with a loss of less than 5% total mass deemed acceptable. Six scaffolds were weighed, placed in the chamber of a friabilator (Vankel 45-2000 Friability Tester, Varian Medical Systems, NSW, Australia,) and tumbled for 100 revolutions over 4 min. Any visible debris present on the surface of the scaffolds was removed using a soft bristled brush before the total weight of the 6 scaffolds post-friability testing was recorded. The test was repeated in triplicate for each type of scaffold and results were expressed as the mean percentage mass loss ±SD.

### C.2.3.5 Hydration

It was hypothesised that the time to equilibrium hydration would influence the rate of release of a loaded cargo from the scaffold materials, as the drug release was expected to occur primarily via diffusion following the ingress of water to dissolve the cargo. Although Gelfoam® had been successfully used in combination with FGF-2 to promote the healing of chronic TM perforation, many patients have required multiple FGF-2 applications, indicating that the effect of FGF-2 had not been sustained over a sufficient time frame to allow for complete tissue regeneration from a single application. Therefore, in order to prolong the release and subsequent activity of loaded cargo, the test scaffold material should ideally have a longer time to equilibrium hydration than the Gelfoam® comparator.

Time to equilibrium hydration was determined for both the test and control scaffolds. The weight of each scaffold was recorded before it was placed into 2 ml of water in a glass vial and stored uncovered, without stirring, at room temperature for up to 21 days. At defined time points (1 h-21 days), the scaffold was removed from the water using forceps, surface water was blotted with Kimwipes (Kimberly-Clark Professional, NSW, Australia) and the wet weight of the scaffold recorded. The time to equilibrium hydration of the scaffold material was defined as the point at which the weight of the scaffold material reached an equilibrium (defined as weight difference of <1% in 3 consecutive measurements).

It is possible that the scaffold material would undergo compression during insertion into the TM perforation. To replicate this scenario, additional scaffolds were compressed using a force of 343 N with a tablet hardness tester (VK 200, Varian Medical Systems, NSW, Australia), prior to the assessment of equilibrium hydration time.

For the pre-formed control scaffolds, cargo would be loaded by soaking the scaffold in a solution of a known cargo concentration. To determine the maximum volume of liquid which may be loaded into the scaffold, the fluid absorptive capacity of the scaffold was calculated. The fluid absorptive capacity of the control scaffold was determined by subtracting the initial weight of the scaffold (prior to hydration) from the weight of the fully hydrated scaffold. The difference in weight was regarded as the fluid absorptive capacity of the scaffold.

### C.2.3.6 Morphology

In order for the scaffold material to promote cellular infiltration, proliferation and differentiation, it should contain uniformly sized pores with an appropriate porous architecture to promote tissue regeneration. The surface morphology and architecture of test and control scaffold materials were visualised by scanning electron microscopy (SEM). All scaffolds were splutter coated with gold prior to visualisation by scanning electron microscope (SU8100, Hitachi, Tokyo, Japan) using an accelerating voltage of 3 kV at 30, 100 and 300× magnifications. The images were processed using ImageJ (National Institutes of Health, Maryland, USA) and the mean pore area, pore diameter, and the overall porosity of each scaffold material was determined from 10 SEM images using the built in “analyse particles” algorithm. A mask was created, highlighting the pores visible in the image. These pores were then treated as “particles” by the algorithm which calculated the average area, diameter and percentage of the image area comprised of these ‘particles’.

### C.2.3.7 Data Analysis

All results are expressed as mean±SD with statistical analysis completed using GraphPad Prism 8 (California, USA) except for the three-way ANOVA, used for analysis of the blank prototype scaffold, which was performed using IBM SPSS (IBM Corporation, New York, USA). Gelfoam® data were analysed by student's t-tests, as indicated in results.

Comparison of the effects of sodium alginate volume, sodium alginate concentration, CaCl2 concentration and mould size on the characteristics of blank prototype scaffolds were analysed using three-way ANOVA, unless otherwise stated, with the sodium alginate volume and concentration condensed into a single parameter as follows. Three sodium alginate volumes (low, medium and high) and three alginate concentrations (1.5, 2 and 3% w/v) were investigated. These parameters were condensed into a single factor by assigning each sodium alginate concentration a number identifier (1.5% w/v=1, 2% w/v=2 and 3% w/v=3) and each alginate volume an alphabetical identifier (low=A, medium=B and high=C). These factors could then be combined into a single parameter (volume and concentration of alginate) with 9 levels. Three-way ANOVA with post-hoc Tukey's test was then performed with sodium alginate concentration/volume, CaCl2 concentration and mould size as the three independent variables.

TABLE 9
Protocol to condense alginate volume and concentration into
a single parameter for statistical analysis of data.

| Sodium | [Sodium alginate] | | |
|---|---|---|---|
| alginate | 1.5% w/v sodium | 2% w/v sodium | 3% w/v sodium |
| Volume | alginate (1) | alginate (2) | alginate (3) |
| Low (A) | A1 | A2 | A3 |
| Medium (B) | B1 | B2 | B3 |
| High (C) | C1 | C2 | C3 |

Comparison of the effects of vehicle substitution on the characteristics of the blank prototype scaffolds was completed by performing a two-way ANOVA with post-hoc Tukey's test applied for a paired comparison of means, unless otherwise stated. This analysis compared the control (water as vehicle) with the other vehicles for all mould sizes.

The effect of vehicle substitution on the characteristics of the FGF-2 loaded scaffold materials was completed using a one-way ANOVA for a paired comparison of means (unless otherwise stated), comparing a single parameter across all vehicles. These scaffolds were additionally compared to their blank vehicle counterparts via a two-way ANOVA with post-hoc Tukey's test applied for a paired comparison of means, unless otherwise stated. For all statistical tests a P value ≤0.05 was considered to be significant.

### C.3 Results

### C.3.1.1. Scaffold Optimisation

Preliminary studies were conducted to determine the optimal combination of sodium alginate and CaCl2 concentrations to produce scaffolds with the most desirable and least variable characteristics. The results of these studies are listed in Table 10. Scaffolds produced from 2% w/v sodium alginate crosslinked with 50 mM CaCl2 were consistent across all parameters. For most parameters measured, the sodium alginate volume did not appear to have any significant impact, however, the diameter of the scaffolds were most reproducible for scaffolds produced from a medium volume of sodium alginate, regardless of mould size. On the basis of these results, a medium volume of 2% w/v sodium alginate crosslinked with 50 mM CaCl2 was chosen as the optimal formulation to be carried through for further evaluation. This formulation fulfilled all the objectives of the optimisation study, with the scaffolds having consistent dimensions and weight, friability of less than 5% and taking more time to reach equilibrium hydration than the Gelfoam® comparator.

TABLE 10
Summary of scaffold characterisation parameters investigated and
the formulation conditions which produced the optimal outcome.

| Parameter | Optimal Formulation | Justification |
|---|---|---|
| Macroscopic | 2 or 3% w/v sodium | Very consistent macroscopic |
| Morphology | alginate, 50 or 100 | morphology observed. |
| | mM CaCl2, medium or | Scaffolds was compact while |
| | high volumes | maintaining an obviously |
| | | porous structure. Scaffold |
| | | edges were well defined |
| | | with a uniform surface |
| | | appearance. |
| Diameter | 2% w/v sodium | Most reproducible results |
| | alginate, 50 mM | observed for this |
| | CaCl2, medium | combination irrespective |
| | volume | of mould size |
| Thickness | 2% w/v sodium | Most reproducible results |
| | alginate, 50 mM | observed for these |
| | CaCl2, all volumes | combinations irrespective |
| | | of mould size |
| Weight | N/A | Very little variation in |
| | | results observed. No |
| | | combination appears |
| | | superior to any other. |
| Friability | 2 or 3% w/v sodium | Friability <5% |
| | alginate, 50 mM | |
| | CaCl2, all volumes | |
| Hydration | 1.5, 2 or 3% sodium | Scaffolds maintained their |
| | alginate 50 mM CaCl2, | structural integrity when |
| | all volumes | hydrated, slower to reach |
| | | equilibrium hydration than |
| | | Gelfoam ®. |

### C.3.2 Characterisation of Prototype Scaffold Materials Prepared Using Different Vehicles to Dissolve the Sodium Alginate

Due to the inherent instability of FGF-2, a significant loss of FGF-2 activity would inevitably occur during the incorporation of FGF-2 into an alginate scaffold without appropriate protein stabilisation. Several FGF-2 stabilisation vehicles of this invention were identified and evaluated for their stabilisation effects against the various thermal and processing stressors FGF-2 would be exposed to during the manufacture, storage and use of the FGF-2 loaded alginate scaffold material. The effect of incorporating these vehicles into the formulation was investigated by characterising the blank prototype alginate scaffold materials prepared using different vehicles to dissolve the sodium alginate. Each scaffold was identified by the vehicle in the formulation (stabilisation vehicles 2-6) with scaffolds prepared with water (vehicle 1) serving as a control.

On the basis of the optimisation data presented above, these scaffolds were prepared using a medium volume (1, 0.5 or 0.1 ml for M12, M24 and M96 moulds respectively) of a 2% w/v sodium alginate solution and crosslinked with 50 mM CaCl2.

C.3.2.1 General Description of the Prototype Scaffolds Prepared with Different Stabilisation Vehicles

The replacement of water with the FGF-2 stabilisation vehicles did not appear to affect the diameter, thickness or macroscopic morphology of the scaffolds. As was observed for the blank prototype alginate scaffolds, the larger the mould size, the larger the resultant diameter of the scaffold material, with the diameter appearing consistent for all scaffolds prepared using the same mould size.

When larger scaffolds were cut into 4 mm discs using a biopsy punch (P4), the scaffold materials appeared less uniform. The edges of cut scaffolds are less well defined, with more loose fibres visible and the compression of the scaffold material at the cutting edge caused the edges of the scaffold to appear thinner than its center.

### C.3.2.2 Diameter

As was observed for the blank alginate scaffolds, the diameter of scaffolds prepared with the different vehicles was mainly determined by mould diameter. The diameter of the M12 scaffolds were similar regardless of the vehicle present in the formulation, with the mean diameter of scaffolds prepared with vehicles 2-6 (17.4±0.3 mm) not significantly different from the control (vehicle 1; 17.3±0.5 mm; P=0.8708). Similarly, the diameter of the M24 and M96 scaffolds were not affected by the substitution of water with a different vehicle, with the mean diameter of scaffolds prepared with vehicles 2-6 (13.2±0.3 and 5.2±0.3 mm respectively) not significantly different from the control (vehicle 1; M24=13.0±0.6 mm; M96=5.2±0.4; P=0.8923 and P=0.9312 respectively). The P4 scaffolds were smallest with a mean diameter of 3.4±0.3 mm. As was observed for the uncut scaffolds, the diameter of P4 scaffolds prepared using vehicles 2-6 were not significantly different from the control (3.7±0.5 mm; P=0.8867).

FIG. 19. Diameter of blank prototype alginate scaffolds prepared using different vehicles to dissolve the alginate. Scaffolds were prepared by crosslinking a medium volume of 2% w/v sodium alginate dissolved in: water (vehicle 1), methylcellulose (MC) 0.05% w/v in water (vehicle 2), alanine 20 mM in water (vehicle 3), human serum albumin (HSA) 1 mg/ml in water (vehicle 4), MC 0.05% w/v and alanine 20 mM in water (vehicle 5) or MC 0.05% w/v and HSA 1 mg/ml in water (vehicle 6) with 50 mM CaCl2 using a 12-, 24- or 96-well culture plate as a mould with the largest scaffolds subsequently cut into small discs using a 4 mm biopsy punch. The volume of sodium alginate used was dependent upon the mould size with a medium volume expected to produce scaffolds with an approximate thickness of 3.0 mm. Each data set represents the mean scaffold diameter ±SD, n=6.

### C.3.2.3 Thickness

As all scaffolds were prepared using a medium volume of sodium alginate, the scaffold thickness was comparable across the M12, M24 and M96 moulds. The mean thickness of M12, M24 and M96 scaffolds were 2.62±0.05, 2.50±0.03 and 2.24±0.06 mm, respectively. The mean thickness of scaffolds prepared with vehicles 2-6 (2.45±0.17 mm) were not significantly different from control (vehicle 1; 2.47±0.21 mm; P=0.9313), indicating that scaffold thickness was not affected by the choice of vehicle. The mean thickness of P4 scaffolds were also very consistent (0.81±0.02 mm) regardless of the vehicle present in the formulation, however, as a result of compression during cutting, they were significantly thinner than their uncut counterparts (2.62±0.05 mm, two-way ANOVA, P<0.0001).

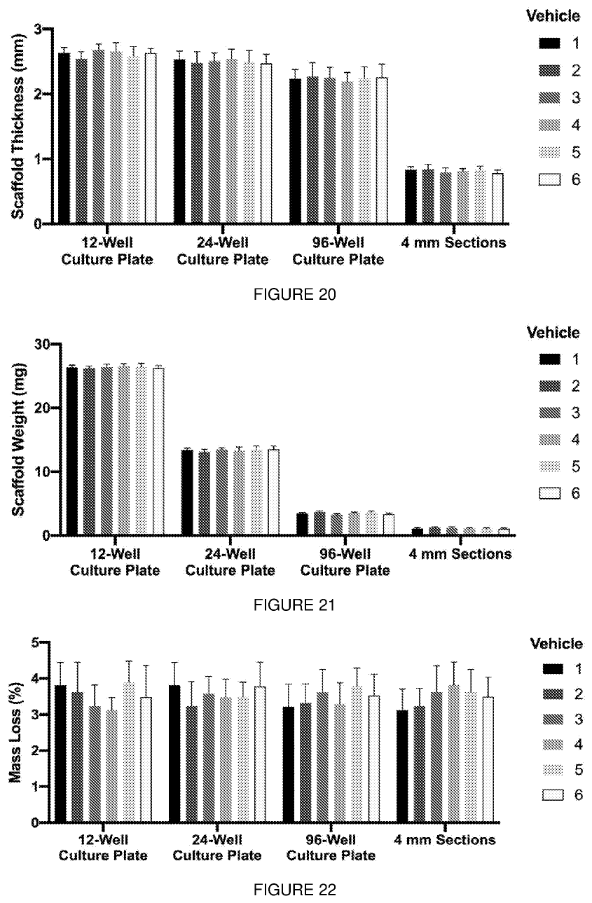

FIG. 20. Thickness of blank prototype alginate scaffolds prepared using different vehicles to dissolve the alginate. Scaffolds were prepared by crosslinking a medium volume of 2% w/v sodium alginate dissolved in: water (vehicle 1), methylcellulose (MC) 0.05% w/v in water (vehicle 2), alanine 20 mM in water (vehicle 3), human serum albumin (HSA) 1 mg/ml in water (vehicle 4), MC 0.05% w/v and alanine 20 mM in water (vehicle 5) or MC 0.05% w/v and HSA 1 mg/ml in water (vehicle 6) with 50 mM CaCl2 using a 12-, 24- or 96-well culture plate as a mould with the largest scaffolds subsequently cut into small discs using a 4 mm biopsy punch. The volume of sodium alginate used was dependent upon the mould size with a medium volume expected to produce scaffolds with an approximate thickness of 3.0 mm. Each data set represents the mean scaffold thickness ±SD, n=6.

### C.3.2.4 Weight

The weight of the M12 scaffolds were similar regardless of the vehicle present in the formulation, with the mean weight of scaffolds prepared with vehicles 2-6 (26.4±0.1 mg) not significantly different from the control (vehicle 1; 26.4±0.4 mg; P=0.9751). Similarly, the weight of the M24 and M96 scaffolds were not affected by the substitution of water with a different vehicle, with the mean weight of scaffolds prepared with vehicles 2-6 (13.4±0.1 and 3.5±0.2 mg respectively) not significantly different from the control (vehicle 1; M24=13.4±0.3 mg; M96=3.5±0.1; P=0.9282 and P=0.9280 respectively). The mean weight of P4 scaffolds prepared using vehicles 2-6 (1.1±0.1 mg) were also not significantly different from the control (1.1±0.2 mg; P=0.9952).

FIG. 21. Weight of blank prototype alginate scaffolds prepared using different vehicles to dissolve the alginate. Scaffolds were prepared by crosslinking a medium volume of 2% w/v sodium alginate dissolved in: water (vehicle 1), methylcellulose (MC) 0.05% w/v in water (vehicle 2), alanine 20 mM in water (vehicle 3), human serum albumin (HSA) 1 mg/ml in water (vehicle 4), MC 0.05% w/v and alanine 20 mM in water (vehicle 5) or MC 0.05% w/v and HSA 1 mg/ml in water (vehicle 6) with 50 mM CaCl2 using a 12-, 24- or 96-well culture plate as a mould with the largest scaffolds subsequently cut into small discs using a 4 mm biopsy punch. The volume of sodium alginate used was dependent upon the mould size with a medium volume expected to produce scaffolds with an approximate thickness of 3.0 mm.

Each data set represents the mean scaffold weight ±SD, n=6.

### C.3.2.5 Friability

Scaffold friability did not appear to be affected by vehicle composition, with all scaffolds demonstrating a similar friability regardless of the vehicle present in the formulation, or the mould size used. The mean friability of M12, M24 and M96 scaffolds prepared with vehicles 2-6 (3.5±0.2% mass loss) was not significantly different from the control (vehicle 1; 3.5±0.4% mass loss; P=0.4629). Additionally, the division of M12 scaffolds into 4 mm discs did not affect the friability of the materials with the mean friability of P4 scaffolds (3.6±0.2% mass loss) not significantly different from M12 (3.5±0.3% mass loss; P=0.3499).

FIG. 22. Friability of blank prototype alginate scaffolds prepared using different vehicles to dissolve the alginate. Scaffolds were prepared by crosslinking a medium volume of 2% w/v sodium alginate dissolved in: water (vehicle 1), methylcellulose (MC) 0.05% w/v in water (vehicle 2), alanine 20 mM in water (vehicle 3), human serum albumin (HSA) 1 mg/ml in water (vehicle 4), MC 0.05% w/v and alanine 20 mM in water (vehicle 5) or MC 0.05% w/v and HSA 1 mg/ml in water (vehicle 6) with 50 mM CaCl2 using a 12-, 24- or 96-well culture plate as a mould with the largest scaffolds subsequently cut into small discs using a 4 mm biopsy punch. The volume of sodium alginate used was dependent upon the mould size with a medium volume expected to produce scaffolds with an approximate thickness of 3.0 mm. Each data set represents the mean % mass loss following tumbling ±SD, n=18.

### C.3.2.6 Hydration

The M12, M24 and M96 alginate scaffolds took between 9.8 and 11.7 days to reach equilibrium hydration with neither the mould size nor vehicle present in the formulation significantly affecting the hydration time of the scaffolds (P=0.9961). In addition, although the cutting of M12 scaffolds to prepare 4 mm discs resulted in thinner scaffolds, the 4 mm discs showed a comparable hydration time to the pre-cut scaffolds. The water uptake-time profile of the alginate scaffolds did not differ upon substitution of water with a different vehicle (data not shown). Water uptake was immediate, with 25% of total water absorbed within the first 7 h, and continued to increase over time with 50% absorption occurring at around 24 h, 75% absorption around 3 days and equilibrium reached at around 11 days.

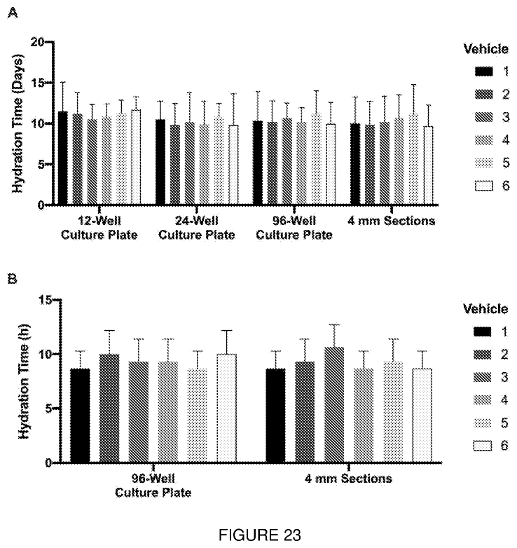

FIG. 23. The effect of different vehicles on the equilibrium hydration time of blank prototype alginate scaffolds prepared using different vehicles to dissolve the alginate. Scaffolds were prepared by crosslinking a medium volume of 2% w/v sodium alginate dissolved in: water (vehicle 1), methylcellulose (MC) 0.05% w/v in water (vehicle 2), alanine 20 mM in water (vehicle 3), human serum albumin (HSA) 1 mg/ml in water (vehicle 4), MC 0.05% w/v and alanine 20 mM in water (vehicle 5) or MC 0.05% w/v and HSA 1 mg/ml in water (vehicle 6) with 50 mM CaCl2 using a 12-, 24- or 96-well culture plate as a mould with the largest scaffolds subsequently cut into small discs using a 4 mm biopsy punch. Non-compressed (A) or scaffolds compressed at a force of 343 N (B) were placed into 2 ml of water and the time to reach equilibrium hydration measured. The time to complete hydration of the discs was defined as the point at which the weight of the disc had not changed by >1% after 3 consecutive measurements. Each data point represents the mean±SD, n=6.

Compression of the M96 and P4 scaffolds at a force of 343 N greatly reduced their hydration time. While the non-compressed scaffolds took a mean time of 10.8 days to reach equilibrium, the compressed scaffolds were fully hydrated at a mean time of 9.3 hours. The hydration time of the compressed scaffolds, like that of the non-compressed scaffolds, was not affected by the mould size or vehicle used to prepare the scaffolds (P=0.5659).

### C.3.2.7 SEM Analysis of Scaffold Morphology

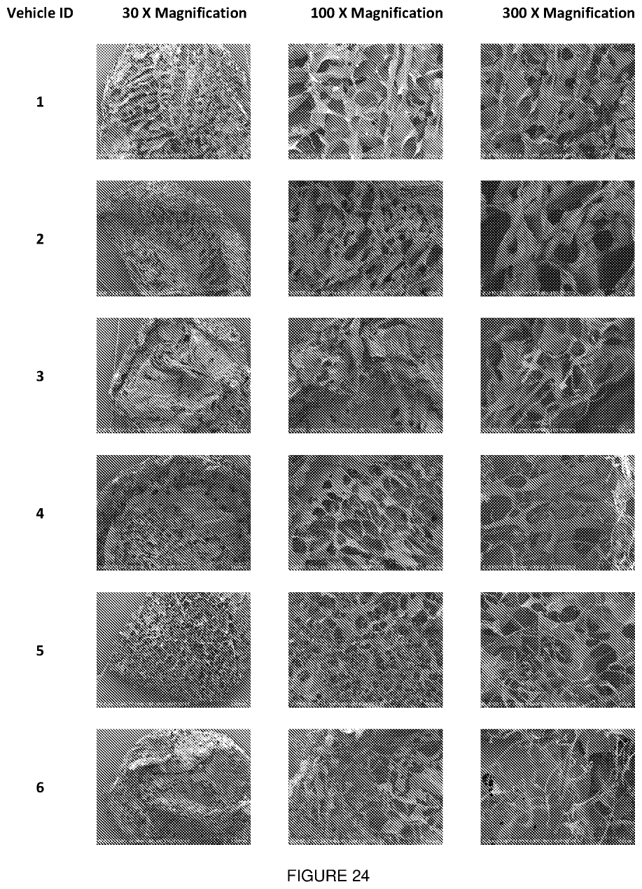

The surface morphology and architecture of M96 alginate scaffold materials produced with various vehicles (1-6) were visualised by SEM. Due to the cost of sample analysis, only M96 non-compressed scaffolds were visualised by SEM. Representative SEM images of each scaffold material are presented in FIG. 24 at various levels of magnification. The surface architecture of the scaffolds shows a highly porous structure, with interconnecting pores. Image analysis suggests that the blank control scaffolds (vehicle 1) have an average pore area of 43507.6 μm2, pore diameter of 118.3 μm and porosity of 84.6%. The substitution of vehicle 1 with vehicles 2-6 did not result in any significant differences in mean pore area (range: 38926.4-42537.8 μm2, P=0.4186), mean pore diameter (range: 104.9-121.2 μm, P=0.7608) or porosity (range: 82.9-84.8%, P=0.8220).

FIG. 24. The surface morphology and architecture of alginate scaffolds prepared using a 96-well culture plate as a mould were visualised by SEM. Scaffolds were prepared by crosslinking 0.1 ml of a 2% w/v sodium alginate dissolved in: water (vehicle 1), methylcellulose (MC) 0.05% w/v in water (vehicle 2), alanine 20 mM in water (vehicle 3), human serum albumin (HSA) 1 mg/ml in water (vehicle 4), MC 0.05% w/v and alanine 20 mM in water (vehicle 5) or MC 0.05% w/v and HSA 1 mg/ml in water (vehicle 6) with 50 mM CaCl2 using a 96-well culture plate as a mould. All scaffolds were splutter coated with gold prior to visualisation by scanning electron microscope using an accelerating voltage of 3 kV at 30, 100 and 300× magnifications.

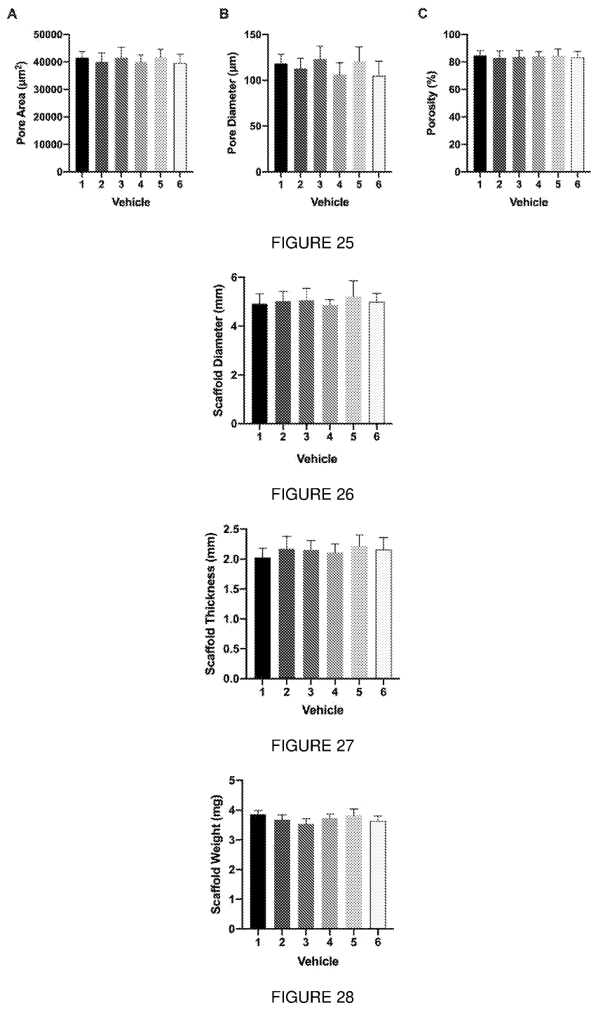

FIG. 25. The mean pore area (A), pore diameter (B) and porosity (C) of scaffolds prepared using a 96-well culture plate as a mould were determined through the analysis of SEM micrographs using the ImageJ software. Scaffolds were prepared by crosslinking 0.1 ml of a 2% w/v sodium alginate dissolved in: water (vehicle 1), methylcellulose (MC) 0.05% w/v in water (vehicle 2), alanine 20 mM in water (vehicle 3), human serum albumin (HSA) 1 mg/ml in water (vehicle 4), MC 0.05% w/v and alanine 20 mM in water (vehicle 5) or MC 0.05% w/v and HSA 1 mg/ml in water (vehicle 6) with 50 mM CaCl2 using a 96-well culture plate as a mould. Data represents the mean±SD values obtained from the analysis of 10 SEM micrographs.

### C.3.3 Characterisation of Prototype FGF-2 Loaded Alginate Scaffold Materials

The effect of incorporating FGF-2 into the alginate scaffold formulation was investigated by repeating the characterisation experiments described above for the scaffold materials loaded with FGF-2 and using vehicles 1-6 as dissolution media. The in vitro release profile of loaded cargo was not determined for this section as it formed part of the functional analysis of FGF-2 scaffold materials above. On the basis that scaffolds produced in different moulds had comparable physical properties, and taking into consideration the prohibitive cost of FGF-2, only the smallest scaffolds were prepared using M96 for this study. Scaffolds were prepared from 0.1 ml of a 2% w/v sodium alginate solution containing 1050 ng FGF-2 and crosslinked with 50 mM CaCl2. Each scaffold was identified by the vehicle in the formulation (stabilisation vehicles 2-6) with scaffolds prepared with water (vehicle 1) serving as a control (Table 11 presented again below for convenience).

TABLE 11
Identification key for FGF-2 stabilisation vehicles.

| Stabilisation | |
|---|---|
| Vehicle ID | Composition of Vehicle |
| 1 | Water (as control) |
| 2 | Methylcellulose (MC; 0.05% w/v) in water |
| 3 | Alanine (20 mM) in water |
| 4 | Human Serum Albumin (HSA; 1 mg/ml) in water |
| 5 | MC (0.05% w/v) and alanine (20 mM) in water |
| 6 | MC (0.05% w/v) and HSA (1 mg/ml) in water |
| 7 | MC (0.05% w/v), alanine (20 mM) and HSA |
| | (1 mg/ml) in water |

### C.3.3.1 General Description

The addition of FGF-2 seemed to produce more compact scaffold materials which were thinner and comprised of smaller pores than their blank (no FGF-2) counterparts. The scaffold diameter and surface appearance did not appear to be affected by the incorporation of FGF-2, with all scaffolds having a uniform appearance.

Scaffolds were prepared by crosslinking 0.1 ml of a 2% w/v sodium alginate dissolved in: water (vehicle 1), methylcellulose (MC) 0.05% w/v in water (vehicle 2), alanine 20 mM in water (vehicle 3), human serum albumin (HSA) 1 mg/ml in water (vehicle 4), MC 0.05% w/v and alanine 20 mM in water (vehicle 5) or MC 0.05% w/v and HSA 1 mg/ml in water (vehicle 6) with 50 mM CaCl2 using a 96-well culture plate as a mould. Each scaffold additionally contained 1050 ng FGF-2.

### C.3.3.2 Diameter

The diameter of scaffolds containing FGF-2 were similar regardless of the vehicle present in the formulation (P=0.7693). Additionally, the mean diameter of the FGF-2 loaded M96 scaffolds (range: 4.9-5.2±0.2-0.6 mm) were not found to be different from their respective blank scaffolds (range: 4.9-5.7±0.2-0.6 mm; P=0.7248).

FIG. 26. Diameter of FGF-2 (1050 ng) loaded prototype alginate scaffolds prepared using different vehicles to dissolve the alginate. Scaffolds were prepared by crosslinking 0.1 ml of a 2% w/v sodium alginate dissolved in: water (vehicle 1), methylcellulose (MC) 0.05% w/v in water (vehicle 2), alanine 20 mM in water (vehicle 3), human serum albumin (HSA) 1 mg/ml in water (vehicle 4), MC 0.05% w/v and alanine 20 mM in water (vehicle 5) or MC 0.05% w/v and HSA 1 mg/ml in water (vehicle 6) with 50 mM CaCl2 using a 96-well culture plate as a mould. Each scaffold additionally contained 1050 ng FGF-2. Each data set represents the mean scaffold diameter ±SD, n=6.

### C.3.3.3 Thickness

As all the FGF-2 loaded M96 alginate scaffolds were prepared using 0.1 ml of sodium alginate solution, they had comparable thickness. The mean thickness of the FGF-2 loaded M96 scaffolds (range: 2.03-2.22±0.14-0.21 mm) were not affected by the choice of vehicle (P=0.5498), and were comparable to the thickness of their blank counterparts (range: 2.19-2.27±0.14-0.21 mm) (P=0.8939).

FIG. 27. Thickness of FGF-2 (1050 ng) loaded prototype alginate scaffolds prepared using different vehicles to dissolve the alginate. Scaffolds were prepared by crosslinking 0.1 ml of a 2% w/v sodium alginate dissolved in: water (vehicle 1), methylcellulose (MC) 0.05% w/v in water (vehicle 2), alanine 20 mM in water (vehicle 3), human serum albumin (HSA) 1 mg/ml in water (vehicle 4), MC 0.05% w/v and alanine 20 mM in water (vehicle 5) or MC 0.05% w/v and HSA 1 mg/ml in water (vehicle 6) with 50 mM CaCl2 using a 96-well culture plate as a mould. Each scaffold additionally contained 1050 ng FGF-2. Each data set represents the mean scaffold thickness ±SD, n=6.

### C.3.3.4 Weight

Scaffold weight was comparable across the FGF-2 loaded M96 alginate scaffolds (FIG. 28). The mean weight of the FGF-2 loaded M96 scaffolds was not affected by the choice of vehicle (P=0.0801) and the incorporation of FGF-2 into the formulation did not have any significant impact on scaffold weight, with the blank scaffolds across 6 vehicles averaging 3.6±0.1 mg, and FGF-2 loaded scaffolds across 6 vehicles averaging 3.5±0.2 mg in weight (P=0.4763).

FIG. 28. Weight of FGF-2 (1050 ng) loaded prototype alginate scaffolds prepared using different vehicles to dissolve the alginate. Scaffolds were prepared by crosslinking 0.1 ml of a 2% w/v sodium alginate dissolved in: water (vehicle 1), methylcellulose (MC) 0.05% w/v in water (vehicle 2), alanine 20 mM in water (vehicle 3), human serum albumin (HSA) 1 mg/ml in water (vehicle 4), MC 0.05% w/v and alanine 20 mM in water (vehicle 5) or MC 0.05% w/v and HSA 1 mg/ml in water (vehicle 6) with 50 mM CaCl2 using a 96-well culture plate as a mould. Each scaffold additionally contained 1050 ng FGF-2. Each data set represents the mean scaffold weight ±SD, n=6.

### C.3.3.5 Friability

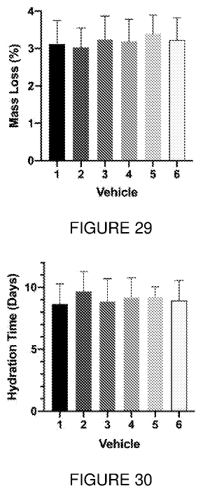

The friability of FGF-2 loaded scaffolds was similar regardless of the vehicle present in the formulation (FIG. 29; P=0.9229) and the mean value for all the FGF-2 scaffolds (3.2±0.1% mass loss) was comparable to that for the blank scaffolds (3.4±0.2% mass loss; P=0.9759).

FIG. 29. Friability of FGF-2 (1050 ng) loaded prototype alginate scaffolds prepared using different vehicles to dissolve the alginate. Scaffolds were prepared by crosslinking 0.1 ml of a 2% w/v sodium alginate dissolved in: water (vehicle 1), methylcellulose (MC) 0.05% w/v in water (vehicle 2), alanine 20 mM in water (vehicle 3), human serum albumin (HSA) 1 mg/ml in water (vehicle 4), MC 0.05% w/v and alanine 20 mM in water (vehicle 5) or MC 0.05% w/v and HSA 1 mg/ml in water (vehicle 6) with 50 mM CaCl2 using a 96-well culture plate as a mould. Each scaffold additionally contained 1050 ng FGF-2. Each data set represents the mean % mass loss following tumbling ±SD, n=18.

### C.3.3.6 Hydration

In order to conserve FGF-2, this study was completed for only non-compressed scaffold materials as these materials took a longer time to reach hydration. The FGF-2 loaded M96 alginate scaffolds took between 8.6 and 9.6 days to reach equilibrium hydration (FIG. 30) with the vehicle present in the formulation not significantly affecting the hydration time of the scaffolds (P=0.8660). The average time to reach equilibrium hydration for the FGF-2 loaded scaffold materials (9.1±1.5 days) was comparable to the blank scaffolds (10.4±1.5 days, P=0.9624). Additionally, the water uptake-time profile of the FGF-2 loaded alginate scaffolds appeared to be similar to those of the blank M96 scaffolds (data not presented).

FIG. 30. The effect of FGF-2 (1050 ng) loading on the equilibrium hydration time of prototype alginate scaffolds prepared using different vehicles to dissolve the alginate. Scaffolds were prepared by crosslinking 0.1 ml of a 2% w/v sodium alginate dissolved in: water (vehicle 1), methylcellulose (MC) 0.05% w/v in water (vehicle 2), alanine 20 mM in water (vehicle 3), human serum albumin (HSA) 1 mg/ml in water (vehicle 4), MC 0.05% w/v and alanine 20 mM in water (vehicle 5) or MC 0.05% w/v and HSA 1 mg/ml in water (vehicle 6) with 50 mM CaCl2 using a 96-well culture plate as a mould. Each scaffold additionally contained 1050 ng FGF-2. Scaffolds were placed into 2 ml of water and the time to reach equilibrium hydration measured. The time to complete hydration of the discs was defined as the point at which the weight of the disc had not changed by >1% after 3 consecutive measurements. Each data point represents the mean±SD, n=6.

### C.3.3.7 SEM Analysis of Scaffold Morphology

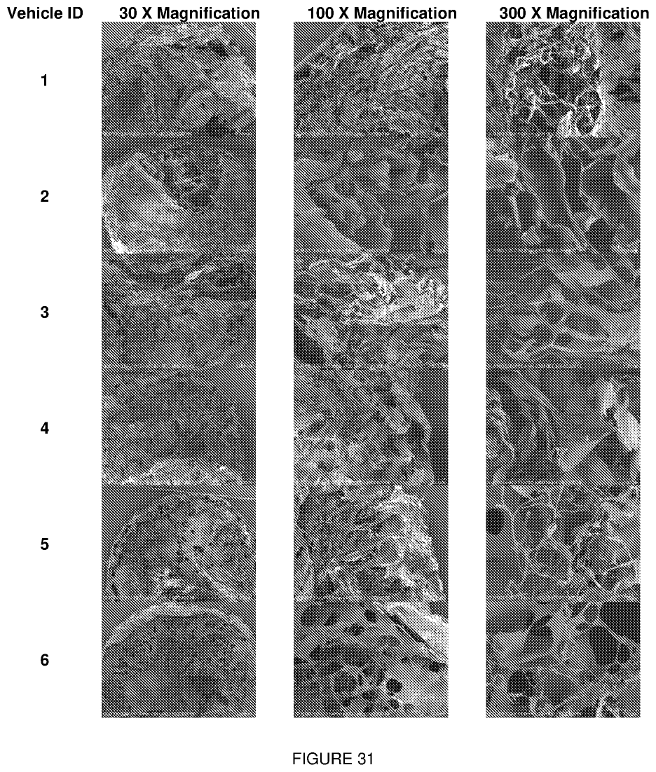

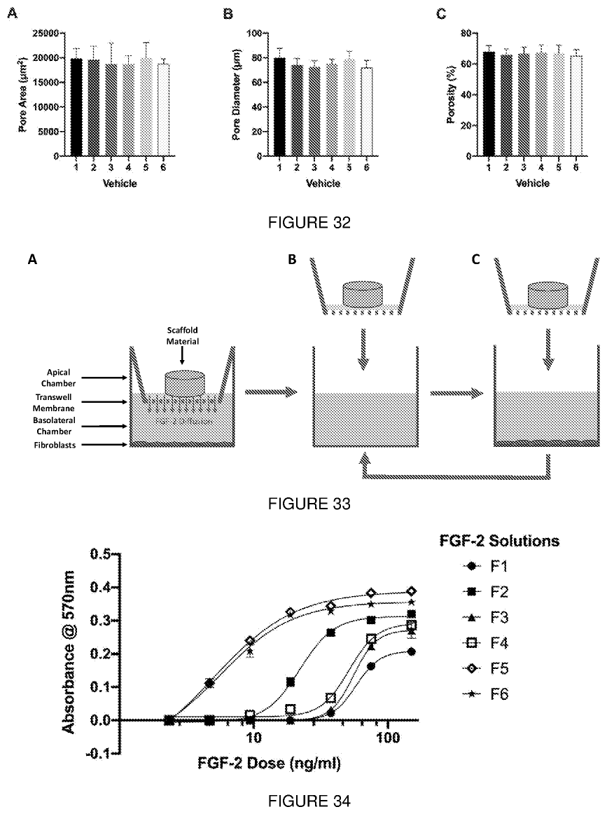

The surface morphology and architecture of FGF-2 loaded M96 alginate scaffold materials produced with various vehicles (1-6) were visualised by SEM. Representative SEM images of each scaffold material are presented in FIG. 31 at various levels of magnification. Similar to the blank scaffold materials, the surface of the FGF-2 loaded scaffolds shows a highly porous structure, with interconnecting pores. The loading of FGF-2 into the alginate scaffolds resulted in a more compact structure, which was confirmed by image analysis (FIG. 32). The average pore areas of the FGF-2-containing scaffolds were in the range of 18698.3 to 19991.2 μm2, while the blank scaffolds had average pore areas that were at least 2-fold higher, in the range of 39601.9 to 41693.3 μm2 (P<0.0001). Similarly, the average pore diameter of the FGF-2-containing scaffolds (range: 72.6 to 79.9 μm) was smaller than those in the blank alginate scaffolds (range: 104.9 to 121.2 μm, P<0.0001) and the porosity of the FGF-2 loaded scaffolds (range: 65.3 to 67.8%) was lower than that of the blank scaffolds (range: 82.9 to 84.8%; P<0.0001).

FIG. 31. The surface morphology and architecture of FGF-2 loaded scaffolds were visualised by SEM. Scaffolds were prepared by crosslinking 0.1 ml of a 2% w/v sodium alginate dissolved in: water (vehicle 1), methylcellulose (MC) 0.05% w/v in water (vehicle 2), alanine 20 mM in water (vehicle 3), human serum albumin (HSA) 1 mg/ml in water (vehicle 4), MC 0.05% w/v and alanine 20 mM in water (vehicle 5) or MC 0.05% w/v and HSA 1 mg/ml in water (vehicle 6) with 50 mM CaCl2 using a 96-well culture plate as a mould. Each scaffold additionally contained 1050 ng FGF-2. All scaffolds were splutter coated with gold prior to visualisation by scanning electron microscope using an accelerating voltage of 3 kV at 30, 100 and 300× magnifications.

FIG. 32. The mean pore area (A), pore diameter (B) and porosity (C) were determined through the analysis of SEM micrographs using the ImageJ software. Scaffolds were prepared by crosslinking 0.1 ml of a 2% w/v sodium alginate dissolved in: water (vehicle 1), methylcellulose (MC) 0.05% w/v in water (vehicle 2), alanine 20 mM in water (vehicle 3), human serum albumin (HSA) 1 mg/ml in water (vehicle 4), MC 0.05% w/v and alanine 20 mM in water (vehicle 5) or MC 0.05% w/v and HSA 1 mg/ml in water (vehicle 6) with 50 mM CaCl2 using a 96-well culture plate as a mould. Each scaffold additionally contained 1050 ng FGF-2. Data represents the mean±SD values obtained from the analysis of 10 SEM micrographs.

### C.4 Discussion

The processes of regeneration and tissue repair consist of a sequence of molecular and cellular events which occur after the onset of a tissue injury. Following the early hemostatic and inflammatory phases of wound healing, new cells migrate towards the wound area, where they undergo proliferation to restore the injured tissue. In most tissues, the underlying cellular structures provide support for the ingression and proliferation of new cells. However, due to the suspension of the TM between two air-filled cavities, there is a distinct lack of a support structure to facilitate the migration of cells and nutrients to the site of perforation. Consequently, a scaffold material capable of adequately supporting cellular ingress and proliferation is preferred to facilitate the repair of chronic TM perforations.

Gelfoam® was used as a comparator for the evaluation of an alginate-based scaffold material which, it was hypothesised, may be customised to enhance FGF-2 efficacy through the addition of various excipient stabilisers, and provide a sustained release of FGF-2.

Optimisation of the alginate-based scaffold materials was achieved through the selection of manufacturing parameters, such as mould size, sodium alginate volume, and sodium alginate and CaCl2 concentrations, and assessing the effects of these parameters on the physical characteristics of the resultant scaffolds. Both the mould size and volume of sodium alginate used to prepare the scaffolds had a direct impact on the dimensions and weight of the resultant scaffold materials. The larger the mould size, the greater the volume of sodium alginate required to produce a scaffold material of a required thickness, and the larger the resultant scaffold dimensions and weight.

Gelfoam® is supplied commercially as a dense sheet of porous, sponge-like material, which is very uniform in appearance. It is easily cut, allowing the size of the scaffold to be modified to fit specific perforations. Although Gelfoam® has shown promising clinical effects, it is difficult to load FGF-2 into the material. In the clinical trials, FGF-2 was loaded into the Gelfoam® material by soaking the Gelfoam® in an aqueous FGF-2 solution.

Both the alginate and Gelfoam® scaffolds underwent more rapid hydration following compression. During the compression of the scaffolds, air, which usually occupied the pore spaces, was expelled. When the compressed scaffold was then placed into water, the pores were quickly filled with the water. By comparison, when an uncompressed scaffold was placed into water, it took much longer for water to disperse throughout the scaffold as the air filling the pore spaces first had to be displaced, and this resulted in a slower hydration time.

The choice of 50 mM CaCl2 as the ideal crosslinking solution for the alginate scaffold material was primarily based on the effect of CaCl2 concentration on the structural integrity of the hydrated scaffold materials. During hydration, the structural integrity of the hydrated scaffold was compromised when the alginate was not sufficiently crosslinked with calcium ions, with all scaffolds prepared using 25 mM CaCl2 as the crosslinking solution undergoing partial disintegration during the study. Similarly, those scaffolds prepared using 100 mM CaCl2 as a crosslinking solution, although structurally sound as uncompressed scaffolds, were found to disintegrate during hydration when they were compressed prior to the hydration study. As the degree of crosslinking increases, alginate scaffolds become more rigid and hence more brittle. It is therefore likely that scaffolds prepared using 100 mM CaCl2 as a crosslinking solution were more brittle than those crosslinked with 50 mM CaCl2 and were more readily damaged during compression. The brittleness of the scaffolds prepared with 100 mM CaCl2 was also reflected in their higher friability compared to scaffolds prepared with a 50 mM CaCl2 crosslinking solution. Scaffolds prepared with 25 mM CaCl2 were also more friable, in this case, the low degree of alginate crosslinking was inadequate to preserve scaffold integrity when stressed, resulting in loosened fibres and subsequent mass loss when the scaffolds were tumbled in the friabilator.

The ideal alginate concentration and volume for the preparation of the optimised scaffold materials were chosen on the basis of reproducibility. Scaffolds produced from a medium volume (1, 0.5 and 0.1 ml, for M12, M24 and M96, respectively) of 2% w/v sodium alginate and crosslinked with 50 mM CaCl2 demonstrated the most consistent dimensions, appearance and friability, indicating that these scaffolds were the most reproducible. In this study, the substitution of water, as the dissolution medium for sodium alginate, with different vehicles did not affect the scaffold characteristics, suggesting that the excipient stabilisers for FGF-2 were not likely to interfere with scaffold production.

The porosity and morphology of the scaffold material are important factors which influence the biocompatibility and wound healing potential of the material. If the pore size is too large, cells are not likely to be retained at the wound site. Conversely, if the pore size is too small, it may be impossible for new cells to penetrate the scaffold. Highly porous materials (60-90% porosity) are advantageous for cellular infiltration and tissue ingress, while pore sizes of 90-160 μm are optimal for fibroblast migration and proliferation. Furthermore, pore sizes of 5-500 μm diameter have been shown to facilitate the successful invasion of new vasculature into the scaffold interior, the absence of which contributes to cell death and tissue necrosis.

The optimised alginate scaffold materials fulfil all the requirements for the promotion of cellular migration, ingression and proliferation, regardless of the FGF-2 stabilisation vehicle present in the formulation or the loading of the scaffold material with FGF-2. The incorporation of FGF-2 into the scaffold materials resulted in decreased pore area, pore diameter and porosity. Heparin-binding proteins such as VEGF and FGF-2 are known to bind to alginate in a similar fashion to heparin, resulting in a more sustained release of these molecules from alginate-based scaffold materials than many other proteins and small molecules. This interaction between FGF-2 and alginate may also be responsible for the smaller pore area, pore diameter and lower porosity of the FGF-2 loaded scaffold materials. The porosity of Gelfoam® on the other hand, was slightly below the recommended range of 60-90%, however the pore diameter met the requirements for the promotion of fibroblast migration and proliferation. It is therefore expected that the FGF-2 loaded alginate-based scaffolds will produce a more sustained release of FGF-2 and provide a more optimal environment for cellular interaction than the FGF-2 loaded Gelfoam® scaffolds.

### D Example 4: In Vitro Evaluation of the Efficacy of FGF-2 Loaded Alginate-Based Scaffold Materials

### D.1 Introduction

Administration of FGF-2 in combination with a scaffold material (e.g. gelatin sponge, hyaluronic acid or bilaminar atelocollagen-silicone membrane) has been shown to produce a faster and higher rate of TM healing than application of the growth factor alone. However, the literature suggests that the bioactivity of FGF-2 in some gelatin-based scaffold formulations is limited to 24-36 hours. A chronic TM perforation typically requires up to 2 weeks, post treatment, to heal, with the remodelling of the fibrous lamina propria taking a further 2-3 weeks. Thus, when a TM perforation fails to heal following a single application of FGF-2, it could be attributed to an insufficient duration of pharmacological effect of the FGF-2. A scaffold that provides prolonged release of functional FGF-2 is therefore highly desirable in the treatment of chronic TM perforations because the insertion of just one such scaffold into the perforation has the potential to provide complete healing of the TM without further medical intervention or patient involvement, making this a highly accessible, economical and predictable treatment modality.

The incorporation of FGF-2 into clinically useful scaffold materials has, however, been severely limited by the inherently poor stability of the protein in solution. FGF-2 degrades so rapidly in aqueous media that it is difficult to retain bioactive protein during the formulation production process. The stabilisation studies presented above showed that this limitation could be overcome by using a combination of stabilisers for the formulation of FGF-2 medicinal products.

An ideal scaffold material will mimic the natural environment for TM regeneration by promoting cellular infiltration, proliferation and differentiation. The characterisation of alginate-based scaffold materials presented above suggests that the prototype scaffolds have the appropriate architecture to support these cellular processes. However, in order for these scaffold prototypes to have clinical translatability, it is essential to determine the threshold FGF-2 loading dose for the promotion of cellular proliferation, and to demonstrate that the FGF-2 is released as a functional protein following its incorporation into the scaffold material. This formed the first aim of the studies presented in this section. The second aim was to demonstrate that the biological effects produced by alginate scaffolds prepared with the stabilised FGF-2 could be sustained over 2 weeks, and were superior to those produced by scaffolds containing non-stabilised FGF-2. The third aim was to demonstrate that the alginate-based scaffold materials were non-cytotoxic.

Gelfoam® was used as a comparator for the evaluation of the alginate-based scaffold materials in this section.

### D.2 Materials and Methods

### D.2.1 Materials

Recombinant human FGF-2 was kindly provided by Essex Bio-Pharmaceutical Co (Zhuhai, China). All cell culture materials, including BALB/c 3T3 murine fibroblast cells, were also kindly provided by Essex Bio. Primary human dermal fibroblast cells (PCS-201-012) were purchased from American Type Culture Collection (ATCC; Virginia, USA). Roswell Park Memorial Institute 1640 (RPMI 1640) media, 0.25% trypsin-EDTA, fetal bovine serum (FBS) and Eagle's minimum essential medium (EMEM) were purchased from Gibco (New York, USA). Tween 20 and sodium carbonate anhydrous were purchased from Shanghai Aladdin Bio-Chem Technology (Shanghai, China). Bovine serum albumin and 3,3′,5,5′-Tetramethylbenzidine (TMB) substrate for ELISA were purchased from West Gene Biotech Inc. (Shanghai, China). Dimethyl sulfoxide (DMSO) and methanol were purchased from Guangzhou Chemical Reagent Factory (Guangzhou, China) and phosphate buffered saline (PBS) tablets were purchased from BBI Life Sciences (Shanghai, China). 3-(4,5-dimethylthiazol-2-yl)-2,5-diphenyltetrazolium bromide (MTT) was purchased from Sigma-Aldrich (Missouri, USA) and sodium bicarbonate was purchased from Macklin Biochemical (Shanghai, China). Deionised water was used throughout. All other materials were the same as those listed above.

### D.2.2 Cell Culture

Murine fibroblast cells and primary human dermal fibroblast cells were seeded at a density of approximately 2.2×10⁶ cells in 10 ml of complete culture media (CCM; RPMI 1640 with 10% FBS for BALB/c 3T3 cells, EMEM with 10% FBS for human dermal fibroblast cells) in a T25 culture flask (Corning, New York, USA), and cultured until confluence in preparation for future studies. Cell cultures were incubated at 37° C. under an atmosphere of 5% CO2. Medium was changed every 2-3 days as required.

### D.2.3 Determination of Threshold FGF-2 Loading Dose

MTT was dissolved in PBS at a concentration of 5 mg/ml, filtered to sterilise, and stored at 4° C. until required for use. Murine fibroblast cells were cultured before 8×103 cells, suspended in 100 μl of CCM, were added to each well of a 96-well culture plate (Corning, New York, USA). The plate was cultured under an atmosphere of 5% CO2 at 37° C. for 24 h. The FGF-2 stock solution (185 μg/ml) was diluted with stabilisation vehicles (1-6) to 600 ng/ml, then serially diluted with test culture media (TCM, RPMI 1640 with 0.2% FBS) to achieve FGF-2 concentrations within the range of 2.3 to 150 ng/ml. The corresponding blank stabilisation vehicles were similarly diluted and served as controls. The diluted test and control solutions were applied at 100 μl/well to the murine fibroblast cells in triplicate. After 48 h culture in 5% CO2 at 37° C., cell viability was determined by the MTT assay. This involved the addition of 25 μl of the MTT solution to each well and incubating the cells for an additional 5 h at 37° C. All liquids were removed and 120 μl of lysis buffer (DMSO:Ethanol, 1:1) was added with shaking to each well (Multiporous Quick Shaker QB-9002, Kylin-Bell Lab Instruments, Jiangsu, China). The absorbances of the wells were measured at 570 nm (reference 630 nm) with a plate reader (iMark, Bio-Rad Laboratories, California, USA). The dose-response profile was determined by subtracting the absorbance values of the corresponding vehicle samples from the absorbance values of the FGF-2 samples, and plotting the net value against the FGF-2 dose with a 4 parameter logistic fit (GraphPad Prism 8, California, USA) which automatically calculated the EC50 value for each of the solutions.

The ideal scaffold would release FGF-2 over a period of at least 14 days to facilitate TM healing. To ensure an adequate amount of FGF-2 would be loaded into each scaffold material, allowing it to act as a reservoir and release sufficient FGF-2 to stimulate cell proliferation over a period of 14 days, the FGF-2 loading dose was calculated as follows:

Loading dose=FGF-2 threshold dose (as determined by the dose-response assay)×14

### D.2.4 FGF-2 Quantification by ELISA

For the experiments in this section, FGF-2 was quantified by ELISA as described previously.

### D.2.5 Preparation of FGF-2 Loaded Scaffold Materials

The FGF-2-loaded alginate scaffold was fabricated using methods similar to those described above. In brief, sodium alginate was dissolved in each of the stabilisation vehicles (vehicle 1-6, see above), and made up to 90% of the final volume required to produce a 2% w/v sodium alginate solution. The remaining 10% of the volume was comprised of FGF-2 in the corresponding stabilisation vehicle (1-6). An FGF-2 stock solution (185 μg/ml in water, FGF-2 content confirmed by ELISA) was diluted with the stabilisation vehicles (1-6) to prepare 105 μg/ml solutions, and these were added (100 μl/ml) to the corresponding sodium alginate solutions to give a final FGF-2 concentration of 10.5 μg/ml. The solutions were vortexed for 2 min to ensure homogeneity, before 100 μl of each solution (containing 1050 ng FGF-2) was added to the wells of a 96-well culture plate (Corning, New York, USA). The plates were frozen at −20° C. for 16 h before 100 μl of a 50 mM CaCl2 solution was added to each well and allowed to crosslink with the thawing alginate for 20 min at room temperature. All scaffolds were washed by completely filling the wells with water, allowing the scaffold to soak for 2 min, then removing all liquid from the well. This process was repeated three times before the scaffolds were immersed in enough water to cover the scaffold surface and frozen at −20° C. for 16 h prior to lyophilisation (VerTis 25L Genesis SQ Super XL-70, SP Scientific, New York, USA) for 24 h. Scaffolds were stored at 4° C. until required for further studies.

For comparison, the commercially available gelatin-based scaffold material, Gelfoam® (Pfizer, New York, USA), was also evaluated. The Gelfoam® scaffold was cut into 4 mm discs using a disposable biopsy punch (Kai Medical, Gifu, Japan). A FGF-2 loading solution (52.5 μg/ml) was produced by diluting the 185 μg/ml FGF-2 stock solution with water, and a Gelfoam® disc was added to 20 μl of the FGF-2 loading solution in a 0.1 ml Eppendorf® tube (Hamburg, Germany). Gentle squeezing of the Gelfoam® disc, with sterile tweezers, encouraged complete uptake of the FGF-2 loading solution, and the protein loading was assumed to be 1050 ng FGF-2 per Gelfoam® disc.

Table 12. Identification key for scaffold materials and solutions.

Scaffolds were prepared by crosslinking various alginate solutions with CaCl2. Scaffolds S1-6 (A) were prepared using alginate dissolved in stabilisation vehicles 1-6 to give the final scaffold composition described below. Scaffolds SF1-6 (B) were prepared using alginate solutions containing FGF-2 (10.5 μg/ml active FGF-2 as determined by ELISA; F1-6) to give the final scaffold composition described below. Scaffolds G1 and GF1 (C) were prepared by soaking 4 mm discs of Gelfoam® in either 20 μl of TCM (test culture media) or FGF-2 in TCM (52.5 μg/ml) to give the final scaffold composition described below. Blank (TCM) and FGF-2 containing (TCM-F) solutions (D) acted as negative and positive controls, respectively.

TABLE 12
A

| Scaffold | |
|---|---|
| ID | Description of Blank Alginate Scaffold |
| S1 | Scaffold prepared by crosslinking a 2% w/v |
| | sodium alginate in water solution with 50 mM |
| | CaCl2 |
| S2 | Scaffold prepared by crosslinking a 2% w/v |
| | sodium alginate and methylcellulose (MC) 0.05% |
| | w/v in water solution with 50 mM CaCl2 |
| S3 | Scaffold prepared by crosslinking a 2% w/v |
| | sodium alginate and alanine 20 mM in water |
| | solution with 50 mM CaCl2 |
| S4 | Scaffold prepared by crosslinking a 2% w/v |
| | sodium alginate and human serum albumin (HSA) |
| | 1 mg/ml in water solution with 50 mM CaCl2 |
| S5 | Scaffold prepared by crosslinking a 2% w/v |
| | sodium alginate, MC 0.05% w/v and alanine 20 mM |
| | in water solution with 50 mM CaCl2 |
| S6 | Scaffold prepared by crosslinking a 2% w/v |
| | sodium alginate, MC 0.05% w/v and HSA 1 mg/ml |
| | in water solution with 50 mM CaCl2 |

B

| Scaffold | |
|---|---|
| ID | Description of FGF-2 Loaded Alginate Scaffold |
| SF1 | Scaffold containing FGF-2 (1050 ng) prepared |
| | by crosslinking a 2% w/v sodium alginate in |
| | water solution with 50 mM CaCl2 |
| SF2 | Scaffold containing FGF-2 (1050 ng) prepared |
| | by crosslinking a 2% w/v sodium alginate and |
| | MC 0.05% w/v in water solution with 50 mM CaCl2 |
| SF3 | Scaffold containing FGF-2 (1050 ng) prepared |
| | by crosslinking a 2% w/v sodium alginate and |
| | alanine 20 mM in water solution with 50 mM CaCl2 |
| SF4 | Scaffold containing FGF-2 (1050 ng) prepared |
| | by crosslinking a 2% w/v sodium alginate and |
| | HSA 1 mg/ml in water solution with 50 mM CaCl2 |
| SF5 | Scaffold containing FGF-2 (1050 ng) prepared |
| | by crosslinking a 2% w/v sodium alginate, MC |
| | 0.05% w/v and alanine 20 mM in water solution |
| | with 50 mM CaCl2 |
| SF6 | Scaffold containing FGF-2 (1050 ng) prepared |
| | by crosslinking a 2% w/v sodium alginate, MC |
| | 0.05% w/v and HSA 1 mg/ml in water solution |
| | with 50 mM CaCl2 |

C

| Scaffold | |
|---|---|
| ID | Description of Gelfoam ® Scaffold |
| G1 | Gelfoam ® disc (4 mm diameter) loaded with 20 μl |
| | of water |
| GF1 | Gelfoam ® disc (4 mm diameter) loaded with 1050 |
| | ng FGF-2 in 20 μl of water |

D

| Solution | |
|---|---|
| ID | Description of Solution |
| TCM | Test culture media (BALB/c 3T3 cells: RPMI 1640 |
| | with 0.2% FBS; human dermal fibroblast cells: |
| | EMEM with 0.2% FBS) |
| TCM-F | Solution of FGF-2 (21 μg/ml) in TCM |

### D.2.6 In Vitro FGF-2 Release Profile

The in vitro release profile of functional FGF-2 from the scaffold materials was determined by placing a FGF-2-loaded scaffold in the apical chamber of a transwell insert (transparent polycarbonate membrane, 24 well, 8.0 μm pore size, Corning, New York, USA), with 0.5 ml of the FGF-2 dilution buffer as dissolution medium in the basolateral chamber. The experiments were conducted at 4° C. to minimise the rate of FGF-2 inactivation once released from the scaffold into the buffer. The entire content of the lower chamber was sampled and replaced with fresh buffer at defined time points (8 h to 16 days). FGF-2 content in the samples was quantified by ELISA as described above.

### D.2.7 Functional Assay of FGF-2 Loaded Scaffold

The biological effect of the scaffolds was examined by determining the extent of proliferation of co-incubated murine (BALB/c 3T3) or human (ATCC PCS-201-012) fibroblast cells. Murine or human fibroblasts were seeded at 2×104 cells suspended in 500 μl of CCM in each well of 24-well culture plates (Corning, New York, USA). After 24 h of incubation, the media was replaced with 500 μl of TCM (murine fibroblasts: RPMI 1640 with 0.2% FBS; human fibroblasts: EMEM with 0.2% FBS) to starve the cells for a further 24 h. TCM was refreshed before transwell inserts (transparent polycarbonate membrane, 24 well, 8.0 μm pore size, Corning, New York, USA) each containing a scaffold (SF1, SF5, SF6 or GF1) with 50 μl of TCM was added to the well (FIG. 33A). The TCM in the apical chamber of the transwell was to ensure FGF-2 released from the scaffold was able to freely diffuse across the transwell membrane to the cells in the basolateral chamber. Parallel experiments were conducted with transwells containing 50 μl of TCM only (negative control) or 50 μl of freshly prepared TCM-F (1050 ng FGF-2/well, positive control). The samples were cultured for 72 h, and the transwell insert was removed. The cellular proliferation following 72 h of exposure to the scaffold materials was measured by the MTT assay, described above. Inserts containing the used scaffolds were placed into new wells containing 500 μl TCM (without cells) and incubated at 37° C. under an atmosphere of 5% CO2 for 4-7 days without any further medium changes (FIG. 33B). A new plate of cells was prepared, as described above, and the inserts containing the used scaffolds were transferred to the new wells containing cells in 500 μl of freshly changed TCM (FIG. 33C). The wells were once again cultured along with the inserts for 72 h before the transwell inserts were removed and the cellular proliferation measured by the MTT assay. Inserts containing the used scaffolds were once again placed into new wells containing 500 μl TCM (without cells) and incubated at 37° C. under an atmosphere of 5% CO2 for 4-7 days without any further medium changes before the process was repeated for a third time. By preparing fresh cells each week, the effects of FGF-2 release for the initial 3 days in every week could be examined. The assay was conducted for up to 3 weeks. Three replicate samples were tested for each sample (n=3).

FIG. 33. Murine (BALB/c 3T3) or human (ATCC PCS-201-012) fibroblast cells were cultured in the basolateral chamber of transwells for 24 h before starvation for a further 24 h. Transwell inserts, each containing a scaffold (SF1, SF5, SF6 or GF1) with 50 μl of TCM in the apical chamber were added to the wells (A). The wells were cultured for 72 h, before the transwell insert was removed and placed into new wells containing 500 μl TCM (without cells) (B). The cells remaining in the basolateral chamber of the wells in (A) were assayed for cellular proliferation using the MTT assay. The inserts in (B) were incubated at 37° C. under an atmosphere of 5% CO2 for 4-7 days. During this period, a plate of new cells (C) was prepared as described previously and the inserts in (B) were transferred to the new wells containing cells in 500 μl of freshly changed TCM (C). The wells in (C) were cultured along with the inserts for 72 h before the transwell inserts were removed and the cellular proliferation measured by the MTT assay. Steps (B) and (C) were repeated to determine the effects of FGF-2 release for the initial 3 days in every week for up to 3 weeks.

D.2.8 Cellular Interaction with the Scaffold Materials

The biocompatibility of the scaffold materials was evaluated using a live/dead cytotoxicity/viability assay. Murine fibroblast cells were cultured before 2×104 cells suspended in 100 μl of CCM were seeded directly onto a scaffold material placed in a well of 24-well culture plates (Corning, New York, USA). The cells were allowed to attach for 1 h under an atmosphere of 5% CO2 at 37° C. Once the cells had attached, 400 μl of CCM was added to each well and the incubation was continued for an additional 48 h. Positive (live) and negative (dead) control samples were also prepared by seeding 2×104 cells suspended in 400 μl of CCM in each well of a 24-well culture plate and culturing for 48 h. The live/dead cell imaging kit (Invitrogen, Oregon, USA) reagents were prepared and added to the scaffolds, following removal of all media, according to the manufacturer's recommendations. Prior to the addition of the imaging reagent to the control samples, the negative (dead) control was prepared by exposing the cells to 400 μl of a 70% methanol in water solution for 30 min to cause cell death. All samples were visualised by fluorescence microscopy (X-Cite Series 120Q, Excelitas Technologies, Massachusetts, USA; Axio Vert A1, Zeiss, Oberkochen, Germany), with live cells stained green (excitation 494 nm, emission 517 nm) and dead cells stained red (excitation 517 nm, emission 617 nm). Images of cellular interaction and attraction to the scaffold materials were captured (MSX10, Micro-shot Technology, Guangzhou, China) and processed (MShot Image Analysis System, Micro-shot Technology, Guangzhou, China). Images of each scaffold were analysed using ImageJ (National Institutes of Health, Maryland, USA) to determine the number of cells within the field of vision that were within 200 μm of scaffold edge, and the number of cells visible within the borders of the scaffold material. Four images were taken of each well (one per quadrant) to ensure coverage of the entire surface of the well. Removal of culture media prior to the addition of Live/Dead cell imaging reagents was presumed to remove any non-adherent cells. Therefore, cells which appeared within the borders of the scaffold material are assumed to be directly interacting with the material in some way. It is, however, impossible to determine whether there is any cellular ingress using this assay. The degree of cell death was considered minimal if less than 10 dead cells were visualised for a particular sample.

### D.2.9 Data Analysis

Results are expressed as mean±SD. Data were analysed by two-way ANOVA with post-hoc Tukey's test (GraphPad Prism 8, California, USA) applied for paired comparison of means, unless stated otherwise. A P value ≤0.05 was considered to be significant.

### D.3 Results

### D.3.1 FGF-2 Dose Optimisation

The addition of escalating doses of stabilised FGF-2 solutions to the BALB/c 3T3 murine fibroblasts resulted in varying proliferative effects (FIG. 34). At the lowest applied FGF-2 dose of 2.3 ng/ml, none of the stabilised FGF-2 solutions showed significant differences in cytoproliferative effects compared to the control (P>0.9999). As the FGF-2 dose was increased, differences in cytoproliferative effects between the solutions became more apparent and, while all solutions showed a plateauing of activity at FGF-dose ≥75 ng/ml, there were differences in maximum activity exhibited by the solutions. At high doses of 75-150 ng/ml, F5 and F6 produced comparable cytoproliferative effects (P>0.9999) that were stronger than the activities of the other FGF-2 solutions. Of the two, F5 was more effective at lower concentrations, its cytoproliferative effect was consistently higher than the other solutions, including F6, over a broader concentration range (9.4-37.5 ng/ml).

F1, F2, F3 and F4 produced comparable proliferative effects at low FGF-2 doses (2.3-9.4 ng/ml, P=0.8655), however F2 and F4 produced superior cytoproliferative effects than both F1 and F3 at doses higher than 18.8 ng/ml, with F2 producing a significantly greater proliferative effect than F4 (P<0.001). At FGF-2 doses greater than 37.5 ng/ml all stabilised FGF-2 solutions produced greater proliferative effects than the control (F1, P<0.0001). On this basis, the cytoproliferative activity of the FGF-2 solutions may be ranked in the following decreasing order: F5>F6>F2>F4>F3>F1. EC50 values determined from the dose-response curve generally support the ranking, with the control (F1) displaying the greatest EC50 (57.88 ng/ml), followed by comparable values for F3 (55.88 ng/ml) and F4 (51.34 ng/ml), then F2 (22.09 ng/ml), F6 (6.11 ng/ml) and F5 (5.61 ng/ml).

FGF-2 doses greater than 75 ng/ml were not associated with significant increases in cytoproliferative activity regardless of stabilisation vehicle. Given the small differences in cellular proliferative effects between FGF-2 doses of 75 and 150 ng/ml and adequate differentiation between the FGF-2 solutions at 75 ng/ml, this concentration was chosen as the threshold FGF-2 dose for maximal cellular activity when considering the loading dose of FGF-2 for the scaffold materials.

FIG. 34. FGF-2 solutions labelled as F1 (water only as vehicle), F2 (water with methylcellulose (MC) 0.05% w/v), F3 (water with alanine 20 mM), F4 (water with human serum albumin (HSA) 1 mg/ml), F5 (water with MC 0.05% w/v and alanine 20 mM) and F6 (water with MC 0.05% w/v and HSA 1 mg/ml) were applied to BALB/c 3T3 murine fibroblasts at escalating doses. Cellular proliferative effects were measured via an MTT assay and the net absorbance (absorbance of culture wells for FGF-solution minus absorbance of culture well for corresponding vehicle) calculated. Each point represents the mean±SD (n=3).

### D.3.2 In Vitro FGF-2 Release Profile

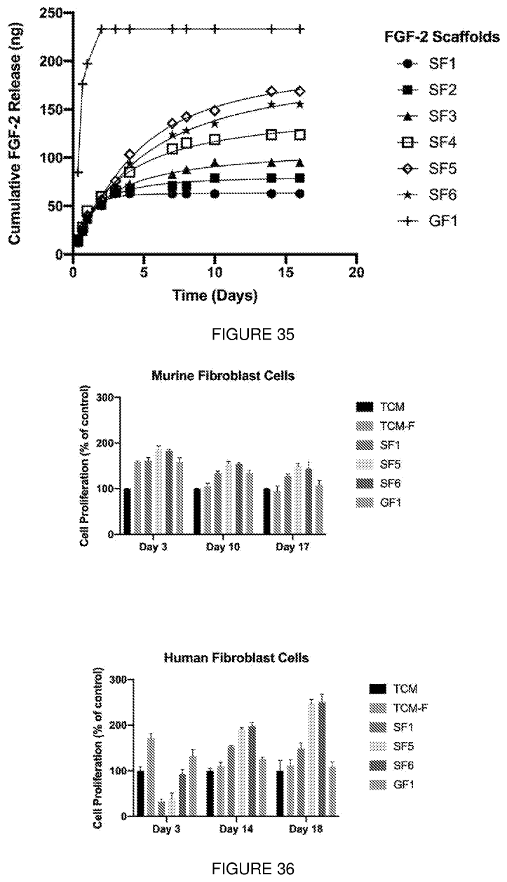

The release of FGF-2 from the alginate scaffold material into the dissolution medium at 4° C. occurred in two phases (FIG. 35). The initial release of FGF-2, over the first two days, was followed by a slower release of FGF-2 for an additional 2-14 days. After 3 days, SF5 and SF6 had released 75.9 and 70.3 ng of FGF-2, respectively, which were significantly more than the other alginate scaffolds (range: 62.7-65.4 ng; P<0.0001). On day 4, SF5 showed greater FGF-2 release than SF6 (103.4 and 94.2 ng respectively, P<0.0001). In the same time period, the release of FGF-2 from SF4 (85.2 ng) was also significantly greater than the release observed from SF2 and SF3 (65.5 and 72.3 ng respectively; P<0.0001) which, in turn, released significantly more FGF-2 than SF1 (62.7 ng) (P<0.0001). In fact, the cumulative release of functional FGF-2 from SF1 appeared to plateau at 62.7 ng from day 3, with no further FGF-2 release detected over the remainder of the study period. By day 14, release of FGF-2 from the other scaffolds also appeared to plateau. From days 7-16, all scaffolds displayed significantly different FGF-2 release profiles from each other, the order of cumulative FGF-2 release being SF5>SF6>SF4>SF3>SF2>SF1.

By comparison, the release of FGF-2 from the GF1 scaffold occurred very rapidly, with 197.3 ng (equivalent to 82.8% of the total cumulative release) of FGF-2 released in the first 24 h. Of all scaffolds, the release of FGF-2 from GF1 appeared to reach a plateau first. The cumulative FGF-2 release from GF1 was 233.3 ng on day 2, with no further FGF-2 release detected over the remainder of the study period.

The actual FGF-2 load in each scaffold could not be determined with confidence due to difficulties in ensuring a complete extraction of the FGF-2 load intact from the scaffold material. Therefore, the cumulative percent release was calculated based on the theoretical FGF-2 load of 1050 ng. On this basis, the cumulative percent FGF-2 released from GF1 was calculated to be 22.2% at the termination of the study at day 16. By comparison, the cumulative FGF-2 release from the alginate-based scaffolds at day 16 was significantly lower with 15.9% of the FGF-2 load released for SF5, 14.7% for SF6, 11.8% for SF4, 9% for SF3, 7.5% for SF2 and only 6.0% for SF1 (one-way ANOVA, P<0.0001).

FIG. 35. Cumulative release of FGF-2 from scaffold materials. Scaffolds SF1-SF6 were prepared by crosslinking a solution of 2% w/v sodium alginate and FGF-2 in: water (SF1), methylcellulose (MC) 0.05% w/v in water (SF2), alanine 20 mM in water (SF3), human serum albumin (HSA) 1 mg/ml in water (SF4), MC 0.05% w/v and alanine 20 mM in water (SF5) or MC 0.05% w/v and HSA 1 mg/ml in water (SF6) with 50 mM CaCl2. The GF1 scaffolds were prepared by soaking a 4 mm disc of Gelfoam® in FGF-2 solution. Each scaffold contained a theoretical FGF-2 load of 1050 ng. The cumulative release of functional FGF-2, into dilution buffer over a period of 16 days, was quantified by ELISA with each data point representing the mean±SD (n=3).

### D.3.3 Functional Assay of FGF-2 Loaded Scaffold Materials

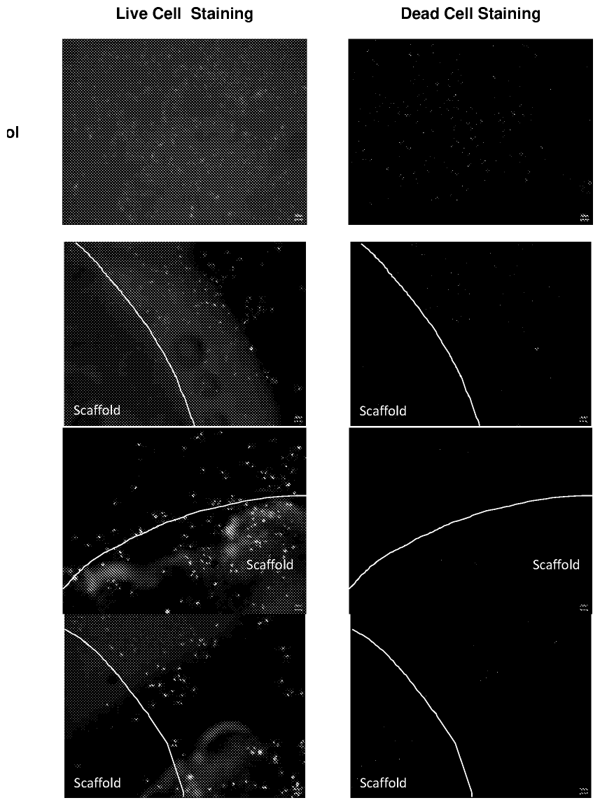

Compared with the other scaffold materials, SF5 and SF6 were observed to produce significantly greater proliferative effects for both the murine and human fibroblast cells over the 17 or 18 day study period, respectively (P=0.0004). However, the timing of the enhanced proliferative effects of the scaffolds were quite different. For the murine fibroblasts, the strongest proliferative effects were seen in the first week of exposure for all FGF-2 samples, with the FGF-2-loaded alginate scaffolds, SF5 and SF6, showing the strongest effects. The SF5 and SF6 scaffolds produced 86.5 and 82.8% greater proliferative effects than control in the first week, which were significantly greater than all other samples (P=0.0082). On the other hand, the FGF-2-loaded alginate scaffolds SF1, SF5 and SF6) appeared to inhibit the proliferation of human fibroblasts in the first week of exposure, although TCM-F and GF1 promoted the proliferation of human fibroblasts during the same period (FIG. 36).

FIG. 36. Comparison of the cytoproliferative effects produced when murine (A) and human (B) fibroblast cells were exposed to FGF-2 loaded (1050 ng) scaffold materials. Scaffolds were intermittently checked for cell proliferative effects using the MTT assay over a 17 (murine fibroblasts) or 18 (human fibroblasts) day period. Each of the FGF-2 (1050 ng) loaded alginate scaffolds, SF1, SF5 and SF6, were prepared by crosslinking a solution of 2% w/v sodium alginate and FGF-2 in: water (SF1), methylcellulose (MC) 0.05% w/v and alanine 20 mM in water (SF5) or MC 0.05% w/v and human serum albumin 1 mg/ml in water (SF6), with 50 mM CaCl2. The GF1 scaffolds were prepared by soaking a 4 mm disc of Gelfoam® in FGF-2 solution to give a theoretical FGF-2 load of 1050 ng. Blank test culture medium (TCM) and free FGF-2 (1050 ng) in TCM (TCM-F) served as controls. Each result represents the average ±SD (n=3).

In the murine cells, there was a trend towards decreasing proliferative effects with increasing exposure time for all test samples, with the proliferative effect of scaffolds SF5 and SF6 dropping by 20.2 and 21.1% over the study period. Despite this, these two scaffolds consistently produced greater proliferative effects than all other samples. The greatest reduction in the proliferative effect produced by SF1, SF5 and SF6 occurred between day 3 and 10 of the study period (24.1, 18.9 and 19.5%, respectively). A further reduction in proliferative effect was observed for all three scaffolds between day 10 and 17, however this decrease was not found to be significant (P=0.9150), indicating that the proliferative effect although lower than day 3 had been sustained.

A 40.5% reduction in the proliferative effects produced by TCM-F were observed by day 10, with no difference between the TCM-F and TCM samples observed on day 10 or 17 (P=0.8846). The GF1 sample had a proliferative effect which was 34.1% greater than blank TCM on day 10 (P<0.0001), but the proliferative effect produced by GF1 was no greater than that of the blank TCM by the endpoint of the study (P=0.6783). These results suggest that although the Gelfoam® material was able to sustain the proliferative effect of FGF-2 over the initial 10 days of this study, neither TCM-F nor GF1 were able to produce a sustained proliferative effect over the entire study period of 17 days.

Similar trends in proliferative response were produced by the TCM-F and GF1 samples for the human fibroblasts. The TCM-F and GF1 samples produced 71.5% and 32.1% greater proliferative effects than control at day 3. However, from day 14 onwards, no difference in cytoproliferative effects were observed between the TCM-F and TCM samples (P>0.9999). The cytoproliferative effect of GF1 remained 22.3% greater than TCM at day 14, however, this was not maintained to the endpoint of the study, with the cytoproliferative effect of GF1 and TCM not significantly different by day 18 (P>0.9999).

By comparison, the proliferative effects of SF5 and SF6 appeared to increase with exposure time for the human fibroblast cells, with both scaffolds producing significantly greater cellular proliferative responses compared to TCM-F, SF1 and GF1 on both day 14 and day 18 (P<0.0001). The proliferative effect of SF1 also increased by 120% between day 3 and day 14 (P<0.0001), but its effects were then maintained at comparable levels between day 14 and day 18 (P>0.9999).

D.3.4 Cellular Interaction with the Scaffold Materials

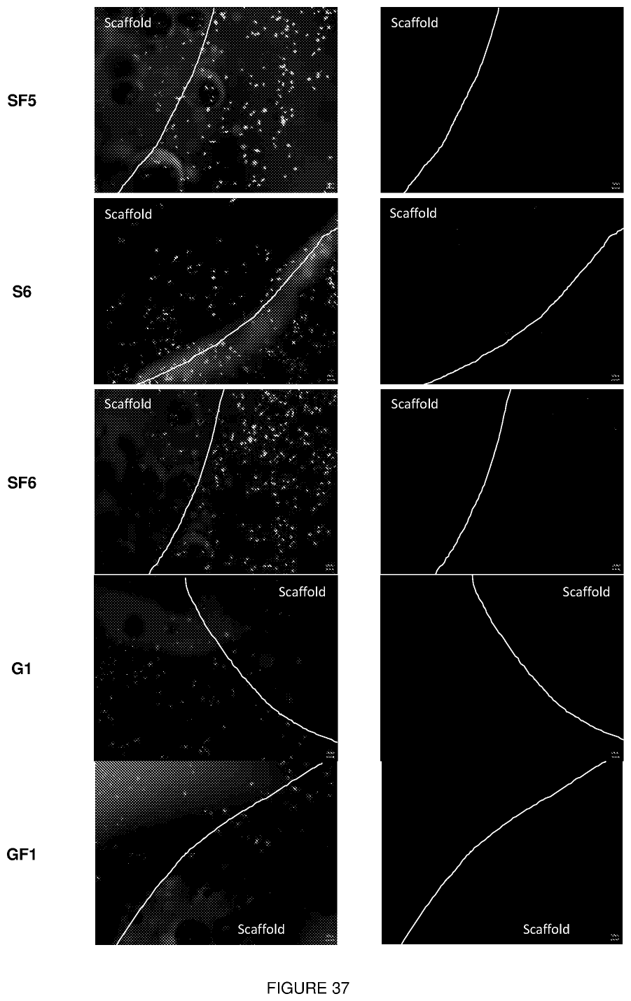

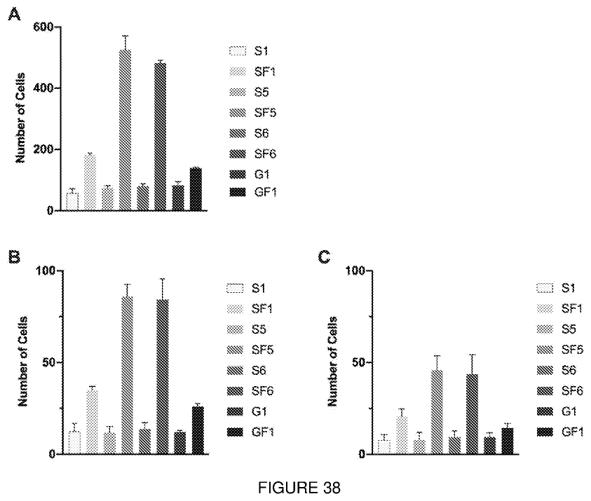

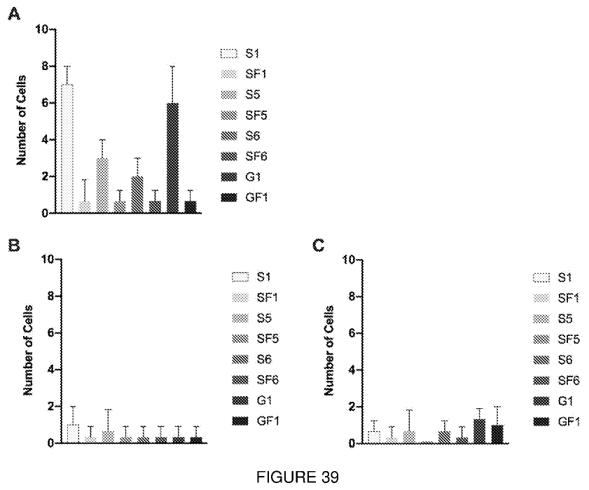

The interaction of murine fibroblast cells with the scaffold materials was observed using a live/dead cell imaging kit, and the scaffold materials were regarded as biocompatible when the cells were able to interact with the scaffold materials with minimal cellular death (FIG. 37). There was no sample, aside from the negative control, that was associated with significant cell death. Both the alginate- and gelatin-based scaffold materials presented with less than 10 dead cells per sample (FIG. 39), indicating biocompatibility between these materials and the murine fibroblast cells. There were, however, varying degrees of cellular interaction with the scaffold materials, depending on whether or not FGF-2 was present, and the composition of the scaffold material. The total number of cells within the field of view was greater for scaffolds containing FGF-2 than the blank scaffold materials (FIG. 38A). SF1 and GF1, which were both prepared with FGF-2 in water, were associated with a significantly greater number of live cells (182 and 139 respectively) within the field of view than the corresponding blank S1 and G1 scaffolds (range: 57-83; one-way ANOVA, P<0.001). The effect of FGF-2 on total cell number was, however, more pronounced with scaffolds SF5 and SF6, which were both prepared with the stabilised FGF-2 solutions, F5 and F6, respectively. There were significantly higher total numbers of live cells within the field of view (526 and 483 respectively) for SF5 and SF6 than for the other scaffold materials (P<0.001).

Comparable numbers of cells were found interacting with SF1 and GF1 (34 and 26 cells, respectively), and these numbers were higher compared with the number of cells found on the corresponding blank scaffolds (P<0.02). Compared with SF1 and GF1, SF5 and SF6 also had a greater mean number (86 and 84, respectively) of live cells directly interacting with the scaffold materials (one-way ANOVA, P<0.001). SF5 and SF6 additionally had comparable numbers of live cells within 200 μm of the scaffold edge (46 and 44 cells, respectively), and these were significantly higher than those observed for the other scaffold materials (P<0.01). SF1, another alginate-based scaffold, also had significant clustering of 21 live cells around its periphery, whereas GF1 had a mean of only 14 live cells in its vicinity, which was not different to the mean number of live cells observed adjacent to the blank scaffolds (P=0.8082).

FIG. 37. Representative stained images of live/dead cells in the interaction between murine fibroblast cells and scaffold materials. Scaffold materials were seeded with 2×104 cells, incubated for 48 h, then washed and stained for live/dead cells. Scaffolds S1-6 and SF1-6 were prepared by crosslinking a solution of 2% w/v sodium alginate dissolved in: water (S1, SF1), methylcellulose (MC) 0.05% w/v in water (S2, SF2), alanine 20 mM in water (S3, SF3), human serum albumin (HSA) 1 mg/ml in water (S4, SF4), MC 0.05% w/v and alanine 20 mM in water (S5, SF5) or MC 0.05% w/v and HSA 1 mg/ml in water (S6, SF6) with 50 mM CaCl2. Scaffolds SF1-6 additionally contained 1050 ng FGF-2. Scaffolds G1 and GF1 were prepared by soaking a 4 mm disc of Gelfoam® in water (G1) or FGF-2 solution (GF1, FGF-2 loading of 1050 ng). Positive (live) and negative (dead) controls were visualised alongside samples to ensure assay specificity. Cells were visualised by fluorescence microscopy, with live cells stained green and dead cells stained red. Diffuse green colouration within the boundary of the scaffold material is artefact produced by the proximity of the scaffold in relation to the wall of the culture plate. All images were captured at 200× magnification, scale bar=100 μm.

FIG. 38. Biocompatibility of scaffold materials as measured by number of live cells interacting with the scaffold materials. Scaffold materials were seeded with 2×104 cells, incubated for 48 h, then washed and stained for live/dead cells. Live cells were selectively visualised using a live/dead cell imaging kit and 4 images of each scaffold were analysed using ImageJ to determine the total number of cells within the field of vision (A), within the borders of the scaffold material (B) or within 200 μm of the scaffold edge (C). Scaffolds S1-6 and SF1-6 were prepared by crosslinking a solution of 2% w/v sodium alginate dissolved in: water (S1, SF1), methylcellulose (MC) 0.05% w/v in water (S2, SF2), alanine 20 mM in water (S3, SF3), human serum albumin (HSA) 1 mg/ml in water (S4, SF4), MC 0.05% w/v and alanine 20 mM in water (S5, SF5) or MC 0.05% w/v) and HSA 1 mg/ml in water (S6, SF6) with 50 mM CaCl2. Scaffolds SF1-6 additionally contained 1050 ng FGF-2. Scaffolds G1 and GF1 were prepared by soaking a 4 mm disc of Gelfoam® in water (G1) or FGF-2 solution (GF1, FGF-2 loading of 1050 ng). Each data point represents the mean number of live cells present ±SD (n=4).

FIG. 39. Cytotoxicity of scaffold materials as measured by number of dead cells interacting with the scaffold materials. Scaffold materials were seeded with 2×104 cells, incubated for 48 h, then washed and stained for live/dead cells. Dead cells were identified using a live/dead cell imaging kit and 4 images of each scaffold were analysed using ImageJ to determine the total number of cells within the field of vision (A), within the borders of the scaffold material (B) or within 200 μm of the scaffold edge (C). Scaffolds S1-6 and SF1-6 were prepared by crosslinking a solution of 2% w/v sodium alginate dissolved in: water (S1, SF1), methylcellulose (MC) 0.05% w/v in water (S2, SF2), alanine 20 mM in water (S3, SF3), human serum albumin (HSA) 1 mg/ml in water (S4, SF4), MC 0.05% w/v and alanine 20 mM in water (S5, SF5) or MC 0.05% w/v) and HSA 1 mg/ml in water (S6, SF6) with 50 mM CaCl2. Scaffolds SF1-6 additionally contained 1050 ng FGF-2. Scaffolds G1 and GF1 were prepared by soaking a 4 mm disc of Gelfoam® in water (G1) or FGF-2 solution (GF1, FGF-2 loading of 1050 ng). Each data point represents the mean number of dead cells present ±SD (n=4).

### D.4 Discussion

The ability of FGF-2 loaded scaffold materials to successfully modulate wound healing is dependent upon many factors, including the level of biological activity, timing of this activity and biocompatibility of the formulation. The biological activity of FGF-2 is, in turn, dependent upon an effective dose being administered. The FGF-2 used in this study, when dispersed in water is quickly inactivated, as evidenced by its short half-life of just 30 minutes at 37° C., a value that was similar to the half-life of 37 min reported in the literature. Therefore, when F1 (FGF-2 in water) was added to the murine fibroblast cells in this study, the FGF-2 would be rapidly inactivated, and its apparent cellular proliferative effects could only be observed when the applied FGF-2 dose was sufficiently high, the threshold in our study was 75 ng/ml. Conversely, the effective stabilisation of the FGF-2 solution by the application of dual stabilisers in F5 and F6 led to cellular proliferative effects being measurable even when the FGF-2 was applied at doses lower than 20 ng/ml.

The FGF-2 threshold dose for promoting the proliferation of the primary human dermal fibroblasts was determined to be 50 ng/ml, and the cellular proliferative effects were measurable at the lowest applied FGF-2 dose of 9.8 μg/ml. These differences in threshold doses could be attributed to the different cell cultures and FGF-2 proteins used for the experiments. FGF-2 is also known for producing different cytoproliferative effects depending on cell types, with one study showing human mesenchyme-derived progenitor cells responding to lower doses of FGF-2 compared with rodent-derived cells. The stabilisation vehicles produced similar cytoproliferative enhancing effects for FGF-2 in the two studies.

In this section, for both the human and murine fibroblasts, it was also observed that the F5 and F6 solutions produced the highest maximal proliferative responses corresponding to the lowest EC50 values, with the proliferative effects of these solutions potentiated approximately 10-fold in comparison to F1. The enhanced cellular proliferative effects of F5 and F6, both of which contained dual stabilisers, were indicative of increased FGF-2 stability at 37° C.

In this study, FGF-2 was incorporated into the alginate scaffolds concurrently with scaffold fabrication. The in vitro FGF-2 release data from our study demonstrated that functional FGF-2 was successfully released from the alginate scaffold materials, and this release was sustained, with functional FGF-2 detectable in the dissolution medium for up to 14 days. By comparison, the release of functional FGF-2 from the Gelfoam® scaffold, GF1, occurred rapidly and was limited to 2 days.

Although the in vitro release of FGF-2 from all alginate-based scaffolds in our study appeared to plateau after 14 days, it is possible that FGF-2 release from the scaffolds had continued beyond that point, but the level of FGF-2 released into the dissolution medium was below the detection limit of the ELISA protocol used for this study, and therefore unable to be quantified. The higher FGF-2 release observed from the scaffolds prepared with solutions SF5 and SF6 might be due to the increased stability of FGF-2 following its release into solution.

For wound healing applications, any entrapped FGF-2 may be advantageous in producing a prolonged chemotactic effect, attracting cells towards the scaffold edge and encouraging cell ingression into the scaffold material. The cellular ingression may not only be helpful in closing the wound, but may result in the remodelling of the scaffold material that then allows the residual FGF-2 to become accessible throughout the wound healing process. This chemotactic effect was apparent during the biocompatibility experiments. The higher density of live cells directly interacting with and surrounding the FGF-2 loaded scaffold materials (SF1, SF5, SF6 and GF1) is most likely due to the chemotactic effect of FGF-2, which attracts cells towards the scaffold material along a concentration gradient.

The sustained release profile and comparatively higher rate of FGF-2 release from SF5 and SF6 appear to correlate with the increased proliferative effects observed when either murine or human fibroblasts were exposed to these scaffolds over a period of 3 weeks. The sustained nature of this effect further demonstrates that these FGF-2 loaded scaffold materials are an improvement over previous FGF-2 scaffold formulations, where bioactivity has been limited to only 24-36 hours. Furthermore, these results confirm the hypothesis proposed above that FGF-2 loaded alginate-based scaffolds would produce a more sustained release of FGF-2 than Gelfoam® scaffolds due to the smaller pore size, lower porosity and potential of FGF-2 binding to alginate.

The biocompatibility of the scaffold materials was investigated using a live/dead cell imaging assay. The interaction of cells with the scaffold materials, with minimal cellular death, indicated that all scaffolds were biocompatible. Although all scaffolds were associated with minimal cellular death, the degree of cellular interaction with the scaffold materials appeared to differ depending on both the presence and absence of FGF-2 and composition of the scaffold material. In this respect, SF5 and SF6 were again superior to the blank scaffolds and other FGF-2 loaded scaffolds (SF1 and GF1) in showing a high number of live cells in the scaffold vicinity and directly interacting with the scaffold materials. This increase in cellular interaction with FGF-2 loaded alginate scaffolds was expected based on the assessment of scaffold morphology, which indicated that the pore area, pore diameter and lower porosity of the FGF-2 loaded scaffold materials would promote cellular interaction to a greater degree than the blank and Gelfoam® scaffolds.

1. A composition comprising: (1) a fibroblast growth factor 2 (FGF-2), analog or variant thereof; and (2) a cellulose-based polymer,
wherein said composition further comprises:
(a) an amino acid;
(b) a serum albumin; or
(c) an amino acid and a serum albumin.

2. The composition of claim 1, wherein the cellulose-based polymer is methyl cellulose (MC).

3. The composition of claim 1, wherein the composition is selected from the group consisting of: a pharmaceutical composition; a cosmetic composition; and a veterinary composition.

4. The composition of claim 1, wherein the analog or variant of FGF-2 has an amino acid sequence homology to human FGF-2 selected from the group consisting of: at least 75% sequence homology; at least 80%; at least 85%; at least 90%; at least 95%; at least 96%; at least 97%; at least 98%; and at least 99%.

5. The composition of claim 1, wherein the amino acid is alanine.

6. The composition of claim 1, wherein the serum albumin is human serum albumin.

7. The composition of claim 1, wherein the FGF-2 is present at a concentration selected from the group consisting of: between 1 ng/ml to 5 mg/ml; between 10 ng/ml to 2 mg/ml; between 100 ng/ml to 1 mg/ml; between 200 ng/ml to 800 ng/ml; and 770 ng/ml.

8. The composition of claim 1, wherein the MC is present at a concentration selected from the group consisting of: between 0.01 to 10%; between 0.01 to 5%; between 0.01% to 1%; and 0.05% w/v.

9. The composition of claim 1, wherein the alanine is present at a concentration selected from the group consisting of: between 1 to 500 mM; and between 10 to 100 mM.

10. The composition of claim 1, wherein the serum albumin is present at a concentration selected from the group consisting of: between 0.1 to 100 mg/ml; between 0.5 to 50 mg/ml; and between 1 mg/ml to 10 mg/ml.

11. The composition of claim 1, wherein the composition is an aqueous solution.

12. The composition of claim 1, wherein the composition is adapted for wound healing.

13. The composition of claim 1, wherein the composition is adapted for tissue growth and repair.

14. A dosage form comprising the composition of claim 1.

15. A method for treating a wound, said method comprising administration to a patient in need thereof a therapeutically effective amount of the dosage form of claim 14.

16. The method of claim 15, where the wound is selected from the group consisting of: tympanic membrane perforations and chronic tympanic membrane perforations.

17. A device, comprising the composition of claim 1 and a wound healing scaffold.

18. The device of claim 17, wherein the wound healing scaffold comprises sodium alginate.

19. (canceled)

20. A method for stablising FGF-2, said method comprising preparing the composition of claim 1.
